SEQUENCE LISTING (718 sequences)

SEQ ID NO: 1 - protein, 8 aa, Lama glama
GRTFSNYA

SEQ ID NO: 2 - protein, 8 aa, Lama glama
GRIFGSYV

SEQ ID NO: 3 - protein, 8 aa, Lama glama
GSTVRVTA

SEQ ID NO: 4 - protein, 8 aa, Lama glama
GFTFSMKW

SEQ ID NO: 5 - protein, 8 aa, Lama glama
RTTFSFNY

SEQ ID NO: 6 - protein, 8 aa, Lama glama
GFTFSTKW

SEQ ID NO: 7 - protein, 8 aa, Lama glama
GRTVGSYV

SEQ ID NO: 8 - protein, 8 aa, Lama glama
GFSFDDYA

SEQ ID NO: 9 - protein, 8 aa, Lama glama
GLTASTYA

SEQ ID NO: 10 - protein, 8 aa, Lama glama
GTTFTIED

SEQ ID NO: 11 - protein, 8 aa, Lama glama
GRIFGTYV

SEQ ID NO: 12 - protein, 8 aa, Lama glama
GRTFGSYV

SEQ ID NO: 13 - protein, 8 aa, Lama glama
GRTFGTYV

SEQ ID NO: 14 - protein, 8 aa, Lama glama
GRTSSNYA

SEQ ID NO: 15 - protein, 8 aa, Lama glama
GFTFSTKW

SEQ ID NO: 16 - protein, 8 aa, Lama glama
GTTSSFVY

SEQ ID NO: 17 - protein, 8 aa, Lama glama
GFTLDGYA

SEQ ID NO: 18 - protein, 8 aa, Lama glama
GRTVGSYV

SEQ ID NO: 19 - protein, 8 aa, Lama glama
GRTVGSYA

SEQ ID NO: 20 - protein, 8 aa, Lama glama
GSTFSITA

SEQ ID NO: 21 - protein, 8 aa, Lama glama
GSTVRVTA

SEQ ID NO: 22 - protein, 8 aa, Lama glama
GSTVRVTA

SEQ ID NO: 23 - protein, 8 aa, Lama glama
GFSFDDYA

SEQ ID NO: 24 - protein, 8 aa, Lama glama
GSTFSITA

SEQ ID NO: 25 - protein, 8 aa, Lama glama
GFTFSTKW

SEQ ID NO: 26 - protein, 8 aa, Lama glama
GLTSSTYV

SEQ ID NO: 27 - protein, 8 aa, Lama glama
GLTHSTYV

SEQ ID NO: 28 - protein, 8 aa, Lama glama
RTTFRING

SEQ ID NO: 29 - protein, 8 aa, Lama glama
GRTFSTYA

SEQ ID NO: 30 - protein, 8 aa, Lama glama
GTIESIND

SEQ ID NO: 31 - protein, 8 aa, Lama glama
GLAASTYA

SEQ ID NO: 32 - protein, 8 aa, Lama glama
GLTSSTYV

SEQ ID NO: 33 - protein, 8 aa, Lama glama
GSTFGRNV

SEQ ID NO: 34 - protein, 8 aa, Lama glama
GTTFSFVY

SEQ ID NO: 35 - protein, 8 aa, Lama glama
GSTFSINV

SEQ ID NO: 36 - protein, 8 aa, Lama glama
GSTFSFNY

SEQ ID NO: 37 - protein, 8 aa, Lama glama
GSRFRISG

SEQ ID NO: 38 - protein, 8 aa, Lama glama
GTTFSIEE

SEQ ID NO: 39 - protein, 8 aa, Lama glama
GTTFSFNY

SEQ ID NO: 40 - protein, 8 aa, Lama glama
GTTFSFVY

SEQ ID NO: 41 - protein, 8 aa, Lama glama
GLTSSTYV

SEQ ID NO: 42 - protein, 8 aa, Lama glama
GLAHSTYV

SEQ ID NO: 43 - protein, 8 aa, Lama glama
GRTFGTYV

SEQ ID NO: 44 - protein, 8 aa, Lama glama
GRTVGSYV

SEQ ID NO: 45 - protein, 8 aa, Lama glama
GSTFPINV

SEQ ID NO: 46 - protein, 8 aa, Lama glama
GRTFSSYR

SEQ ID NO: 47 - protein, 8 aa, Lama glama
GRTVGSYV

SEQ ID NO: 48 - protein, 8 aa, Lama glama
GLTASTYV

SEQ ID NO: 49 - protein, 8 aa, Lama glama
GRTVGSYA

SEQ ID NO: 50 - protein, 8 aa, Lama glama
GFTFSTKW

SEQ ID NO: 51 - protein, 8 aa, Lama glama
GTTFDMNS

SEQ ID NO: 52 - protein, 8 aa, Lama glama
GFTFSTKW

SEQ ID NO: 53 - protein, 8 aa, Lama glama
GRTFGTYV

SEQ ID NO: 54 - protein, 8 aa, Lama glama
GRPISAYT

SEQ ID NO: 55 - protein, 8 aa, Lama glama
GRVFGTYV

SEQ ID NO: 56 - protein, 8 aa, Lama glama
GSTFSITA

SEQ ID NO: 57 - protein, 8 aa, Lama glama
GRTIGSYV

SEQ ID NO: 58 - protein, 8 aa, Lama glama
GRTFSSGA

SEQ ID NO: 59 - protein, 8 aa, Lama glama
GFTLDDYA

SEQ ID NO: 60 - protein, 8 aa, Lama glama
GFTLDDYA

SEQ ID NO: 61 - protein, 8 aa, Lama glama
GTIESIND

SEQ ID NO: 62 - protein, 8 aa, Lama glama
GTTFSFVY

SEQ ID NO: 63 - protein, 9 aa, Lama glama
GRTFVGSYV

SEQ ID NO: 64 - protein, 8 aa, Lama glama
GRTFGSYV

SEQ ID NO: 65 - protein, 8 aa, Lama glama
GFTFDDYA

SEQ ID NO: 66 - protein, 8 aa, Lama glama
GRTVGSYV

SEQ ID NO: 67 - protein, 8 aa, Lama glama
GTTFSFVY

SEQ ID NO: 68 - protein, 8 aa, Lama glama
GSTFSITA

SEQ ID NO: 69 - protein, 8 aa, Lama glama
GLTSSTYA

SEQ ID NO: 70 - protein, 8 aa, Lama glama
GTTSSLNL

SEQ ID NO: 71 - protein, 8 aa, Lama glama
GRTFSSGA

SEQ ID NO: 72 - protein, 8 aa, Lama glama
GTTFTIED

SEQ ID NO: 73 - protein, 8 aa, Lama glama
GTTFSFVY

SEQ ID NO: 74 - protein, 8 aa, Lama glama
GSTFNFRI

SEQ ID NO: 75 - protein, 8 aa, Lama glama
GRTSSNYN

SEQ ID NO: 76 - protein, 8 aa, Lama glama
GTTFSFVY

SEQ ID NO: 77 - protein, 8 aa, Lama glama
GTTSSLNL

SEQ ID NO: 78 - protein, 8 aa, Lama glama
GLTASTYA

SEQ ID NO: 79 - protein, 8 aa, Lama glama
GFTFSTKW

SEQ ID NO: 80 - protein, 8 aa, Lama glama
GSTFNFNI

SEQ ID NO: 81 - protein, 8 aa, Lama glama
GTTFSFNY

SEQ ID NO: 82 - protein, 8 aa, Lama glama
GSIFTTAT

SEQ ID NO: 83 - protein, 8 aa, Lama glama
GTTFDMNS

SEQ ID NO: 84 - protein, 8 aa, Lama glama
GSIGRLNV

SEQ ID NO: 85 - protein, 8 aa, Lama glama
GSTFSFNY

SEQ ID NO: 86 - protein, 8 aa, Lama glama
GTTFSFVY

SEQ ID NO: 87 - protein, 9 aa, Lama glama
GRTFVGTYV

SEQ ID NO: 88 - protein, 8 aa, Lama glama
GTTFDMNS

SEQ ID NO: 89 - protein, 8 aa, Lama glama
GFTFSTKW

SEQ ID NO: 90 - protein, 8 aa, Lama glama
GRTFSSGA

SEQ ID NO: 91 - protein, 9 aa, Lama glama
GRTFVGTYV

SEQ ID NO: 92 - protein, 8 aa, Lama glama
GFTLDDYA

SEQ ID NO: 93 - protein, 8 aa, Lama glama
GRTVGTYV

SEQ ID NO: 94 - protein, 8 aa, Lama glama
GSTVRLTA

SEQ ID NO: 95 - protein, 9 aa, Lama glama
GRTFVGSYV

SEQ ID NO: 96 - protein, 8 aa, Lama glama
GFTFSTKW

SEQ ID NO: 97 - protein, 8 aa, Lama glama
GRTVGSYA

SEQ ID NO: 98 - protein, 8 aa, Lama glama
GSTFSIDA

SEQ ID NO: 99 - protein, 8 aa, Lama glama
GNTFGRNV

SEQ ID NO: 100 - protein, 8 aa, Lama glama
GRTFGSYV

SEQ ID NO: 101 - protein, 8 aa, Lama glama
GTIESIND

SEQ ID NO: 102 - protein, 8 aa, Lama glama
GLTASTYA

SEQ ID NO: 103 - protein, 9 aa, Lama glama
GRTFVGSYV

SEQ ID NO: 104 - protein, 8 aa, Lama glama
GRTVGSYA

SEQ ID NO: 105 - protein, 8 aa, Lama glama
GLTASTYA

SEQ ID NO: 106 - protein, 8 aa, Lama glama
GRTVGSYV

SEQ ID NO: 107 - protein, 8 aa, Lama glama
GSTVRVTA

SEQ ID NO: 108 - protein, 8 aa, Lama glama
GFTLDDYA

SEQ ID NO: 109 - protein, 8 aa, Lama glama
GTTFTIED

SEQ ID NO: 110 - protein, 8 aa, Lama glama
GFTFSTKW

SEQ ID NO: 111 - protein, 8 aa, Lama glama
GFTFSNKW

SEQ ID NO: 112 - protein, 8 aa, Lama glama
TRTVGTYV

SEQ ID NO: 113 - protein, 9 aa, Lama glama
GRTFVGTYV

SEQ ID NO: 114 - protein, 8 aa, Lama glama
GFTFSTKW

SEQ ID NO: 115 - protein, 8 aa, Lama glama
GFTLDDYA

SEQ ID NO: 116 - protein, 8 aa, Lama glama
GSTSSIVI

SEQ ID NO: 117 - protein, 9 aa, Lama glama
GRTFVGSYV

SEQ ID NO: 118 - protein, 8 aa, Lama glama
GLTASTYA

SEQ ID NO: 119 - protein, 8 aa, Lama glama
TSTLDFAI

SEQ ID NO: 120 - protein, 8 aa, Lama glama
GRTVGSYA

SEQ ID NO: 121 - protein, 8 aa, Lama glama
GRTVGSYA

SEQ ID NO: 122 - protein, 8 aa, Lama glama
GSTFSFNY

SEQ ID NO: 123 - protein, 8 aa, Lama glama
RTTFSFNY

SEQ ID NO: 124 - protein, 9 aa, Lama glama
GRTFVGSYV

SEQ ID NO: 125 - protein, 8 aa, Lama glama
GSTVRVTA

SEQ ID NO: 126 - protein, 8 aa, Lama glama
GLTSSTYV

SEQ ID NO: 127 - protein, 8 aa, Lama glama
GTTFTIED

SEQ ID NO: 128 - protein, 8 aa, Lama glama
GTTFSFVY

SEQ ID NO: 129 - protein, 8 aa, Lama glama
GSISSIND

SEQ ID NO: 130 - protein, 9 aa, Lama glama
GRTFVGSYV

SEQ ID NO: 131 - protein, 8 aa, Lama glama
GTTFSFVY

SEQ ID NO: 132 - protein, 8 aa, Lama glama
GFTFSTKW

SEQ ID NO: 133 - protein, 8 aa, Lama glama
GTTFDMNS

SEQ ID NO: 134 - protein, 8 aa, Lama glama
GSTFSVTA

SEQ ID NO: 135 - protein, 8 aa, Lama glama
GFSLDYYA

SEQ ID NO: 136 - protein, 8 aa, Lama glama
GLTSSTYV

SEQ ID NO: 137 - protein, 8 aa, Lama glama
GLTSSTYV

SEQ ID NO: 138 - protein, 8 aa, Lama glama
GRTVGTYA

SEQ ID NO: 139 - protein, 8 aa, Lama glama
GFTLDDYA

SEQ ID NO: 140 - protein, 8 aa, Lama glama
GRTVGTYV

SEQ ID NO: 141 - protein, 8 aa, Lama glama
GRTVGSYA

SEQ ID NO: 142 - protein, 8 aa, Lama glama
GTTFSFVY

SEQ ID NO: 143 - protein, 8 aa, Lama glama
GRTFGTYV

SEQ ID NO: 144 - protein, 8 aa, Lama glama
GTTFSANT

SEQ ID NO: 145 - protein, 8 aa, Lama glama
GSTFGRNV

SEQ ID NO: 146 - protein, 8 aa, Lama glama
GTTSSLNL

SEQ ID NO: 147 - protein, 8 aa, Lama glama
GFTFSTKW

SEQ ID NO: 148 - protein, 8 aa, Lama glama
GTTSSLNL

SEQ ID NO: 149 - protein, 8 aa, Lama glama
GSTFSINV

SEQ ID NO: 150 - protein, 8 aa, Lama glama
GSTVRVTA

SEQ ID NO: 151 - protein, 8 aa, Lama glama
GFTLDDYA

SEQ ID NO: 152 - protein, 8 aa, Lama glama
ASTSSINL

SEQ ID NO: 153 - protein, 8 aa, Lama glama
GRTFGSYV

SEQ ID NO: 154 - protein, 8 aa, Lama glama
GNTFGDNV

SEQ ID NO: 155 - protein, 8 aa, Lama glama
GSTFSINV

SEQ ID NO: 156 - protein, 8 aa, Lama glama
GSTFSINV

SEQ ID NO: 157 - protein, 8 aa, Lama glama
GSTFGRNV

SEQ ID NO: 158 - protein, 8 aa, Lama glama
GSTFSINV

SEQ ID NO: 159 - protein, 8 aa, Lama glama
ISRDDTST

SEQ ID NO: 160 - protein, 8 aa, Lama glama
SSRSTGNT

SEQ ID NO: 161 - protein, 7 aa, Lama glama
IDNGGRP

SEQ ID NO: 162 - protein, 8 aa, Lama glama
IDTSGSST

SEQ ID NO: 163 - protein, 7 aa, Lama glama
ISSGGIT

SEQ ID NO: 164 - protein, 8 aa, Lama glama
IDASGGST

SEQ ID NO: 165 - protein, 8 aa, Lama glama
SSRSTGNT

SEQ ID NO: 166 - protein, 7 aa, Lama glama
ITSDGST

SEQ ID NO: 167 - protein, 7 aa, Lama glama
IDWKGDT

SEQ ID NO: 168 - protein, 6 aa, Lama glama
ISGGNT

SEQ ID NO: 169 - protein, 8 aa, Lama glama
VSRSTGST

SEQ ID NO: 170 - protein, 8 aa, Lama glama
ASRSTGNT

SEQ ID NO: 171 - protein, 8 aa, Lama glama
FSRSTGST

SEQ ID NO: 172 - protein, 8 aa, Lama glama
ISRDGADT

SEQ ID NO: 173 - protein, 8 aa, Lama glama
IDASGGST

SEQ ID NO: 174 - protein, 7 aa, Lama glama
ISSDGST

SEQ ID NO: 175 - protein, 8 aa, Lama glama
IATSDGST

SEQ ID NO: 176 - protein, 8 aa, Lama glama
SSRSTGNT

SEQ ID NO: 177 - protein, 8 aa, Lama glama
FSRSTGNT

SEQ ID NO: 178 - protein, 7 aa, Lama glama
VANDGST

SEQ ID NO: 179 - protein, 7 aa, Lama glama
IDNGGST

SEQ ID NO: 180 - protein, 7 aa, Lama glama
IDNGGST

SEQ ID NO: 181 - protein, 7 aa, Lama glama
ITSDGST

SEQ ID NO: 182 - protein, 7 aa, Lama glama
IANDGST

SEQ ID NO: 183 - protein, 8 aa, Lama glama
IDAGGGST

SEQ ID NO: 184 - protein, 7 aa, Lama glama
IDSRGDT

SEQ ID NO: 185 - protein, 7 aa, Lama glama
IDSSGDT

SEQ ID NO: 186 - protein, 7 aa, Lama glama
ASSDGPT

SEQ ID NO: 187 - protein, 8 aa, Lama glama
IRWDGGST

SEQ ID NO: 188 - protein, 7 aa, Lama glama
ITSGGST

SEQ ID NO: 189 - protein, 7 aa, Lama glama
IDWKGDS

SEQ ID NO: 190 - protein, 7 aa, Lama glama
VDWSGDR

SEQ ID NO: 191 - protein, 7 aa, Lama glama
ITTSGST

SEQ ID NO: 192 - protein, 7 aa, Lama glama
ISSDGST

SEQ ID NO: 193 - protein, 11 aa, Lama glama
LVAFIGRTGTT

SEQ ID NO: 194 - protein, 7 aa, Lama glama
ISSGGIT

SEQ ID NO: 195 - protein, 7 aa, Lama glama
ITRGGST

SEQ ID NO: 196 - protein, 7 aa, Lama glama
ISGGGNT

SEQ ID NO: 197 - protein, 7 aa, Lama glama
ISSGGIT

SEQ ID NO: 198 - protein, 7 aa, Lama glama
ISSDGST

SEQ ID NO: 199 - protein, 7 aa, Lama glama
IDSRGDT

SEQ ID NO: 200 - protein, 7 aa, Lama glama
IDWSGDT

SEQ ID NO: 201 - protein, 8 aa, Lama glama
FSRSTGNT

SEQ ID NO: 202 - protein, 8 aa, Lama glama
NSRSTGNT

SEQ ID NO: 203 - protein, 10 aa, Lama glama
VAFIGRSGTT

SEQ ID NO: 204 - protein, 8 aa, Lama glama
ITGDGNSI

SEQ ID NO: 205 - protein, 8 aa, Lama glama
SSRSTGNT

SEQ ID NO: 206 - protein, 7 aa, Lama glama
IDWRGEP

SEQ ID NO: 207 - protein, 8 aa, Lama glama
SSRSTGNT

SEQ ID NO: 208 - protein, 8 aa, Lama glama
IDASGGST

SEQ ID NO: 209 - protein, 7 aa, Lama glama
ISIGGDT

SEQ ID NO: 210 - protein, 8 aa, Lama glama
IDASGGST

SEQ ID NO: 211 - protein, 8 aa, Lama glama
FSRSTGST

SEQ ID NO: 212 - protein, 8 aa, Lama glama
ITSSAGTT

SEQ ID NO: 213 - protein, 8 aa, Lama glama
FSRSTGHI

SEQ ID NO: 214 - protein, 7 aa, Lama glama
IANDGST

SEQ ID NO: 215 - protein, 8 aa, Lama glama
FSRSTGHI

SEQ ID NO: 216 - protein, 8 aa, Lama glama
IRWSDERK

SEQ ID NO: 217 - protein, 8 aa, Lama glama
IASSDGST

SEQ ID NO: 218 - protein, 8 aa, Lama glama
IATSDGST

SEQ ID NO: 219 - protein, 7 aa, Lama glama
ITSGGST

SEQ ID NO: 220 - protein, 7 aa, Lama glama
ISRDGST

SEQ ID NO: 221 - protein, 8 aa, Lama glama
SSRSTGNT

SEQ ID NO: 222 - protein, 8 aa, Lama glama
FSRSTGST

SEQ ID NO: 223 - protein, 7 aa, Lama glama
RSTDGST

SEQ ID NO: 224 - protein, 8 aa, Lama glama
NSRSTGNT

SEQ ID NO: 225 - protein, 7 aa, Lama glama
ISSDGST

SEQ ID NO: 226 - protein, 7 aa, Lama glama
IANDGST

SEQ ID NO: 227 - protein, 7 aa, Lama glama
IDWRGST

SEQ ID NO: 228 - protein, 7 aa, Lama glama
MTSDGRT

SEQ ID NO: 229 - protein, 8 aa, Lama glama
IRWSDERK

SEQ ID NO: 230 - protein, 6 aa, Lama glama
ISGGNT

SEQ ID NO: 231 - protein, 7 aa, Lama glama
ISSDGST

SEQ ID NO: 232 - protein, 7 aa, Lama glama
INSGGSA

SEQ ID NO: 233 - protein, 8 aa, Lama glama
IPWTGSTT

SEQ ID NO: 234 - protein, 7 aa, Lama glama
ISSDGST

SEQ ID NO: 235 - protein, 7 aa, Lama glama
MTSDGRT

SEQ ID NO: 236 - protein, 7 aa, Lama glama
IDWRGDT

SEQ ID NO: 237 - protein, 8 aa, Lama glama
IDASGGST

SEQ ID NO: 238 - protein, 7 aa, Lama glama
INSGGSA

SEQ ID NO: 239 - protein, 7 aa, Lama glama
ISSGGST

SEQ ID NO: 240 - protein, 7 aa, Lama glama
ITTGGST

SEQ ID NO: 241 - protein, 7 aa, Lama glama
ISIGGDT

SEQ ID NO: 242 - protein, 7 aa, Lama glama
ISSDNKK

SEQ ID NO: 243 - protein, 7 aa, Lama glama
ISSGGIT

SEQ ID NO: 244 - protein, 7 aa, Lama glama
ISSDGST

SEQ ID NO: 245 - protein, 8 aa, Lama glama
SSRSTGNT

SEQ ID NO: 246 - protein, 7 aa, Lama glama
ISMGGDT

SEQ ID NO: 247 - protein, 8 aa, Lama glama
IDAGGTST

SEQ ID NO: 248 - protein, 8 aa, Lama glama
IRWSDERT

SEQ ID NO: 249 - protein, 8 aa, Lama glama
SSRSTGNT

SEQ ID NO: 250 - protein, 8 aa, Lama glama
IATSDGST

SEQ ID NO: 251 - protein, 8 aa, Lama glama
FSRSTGST

SEQ ID NO: 252 - protein, 7 aa, Lama glama
IDNGGGT

SEQ ID NO: 253 - protein, 8 aa, Lama glama
SSRSTGNT

SEQ ID NO: 254 - protein, 8 aa, Lama glama
IDASGGST

SEQ ID NO: 255 - protein, 8 aa, Lama glama
FSRSTGPT

SEQ ID NO: 256 - protein, 7 aa, Lama glama
ISSGGRS

SEQ ID NO: 257 - protein, 7 aa, Lama glama
ITSSGST

SEQ ID NO: 258 - protein, 8 aa, Lama glama
ASRSTGNT

SEQ ID NO: 259 - protein, 7 aa, Lama glama
VSSSGMT

SEQ ID NO: 260 - protein, 7 aa, Lama glama
IDWKGDT

SEQ ID NO: 261 - protein, 8 aa, Lama glama
SSRSTGNT

SEQ ID NO: 262 - protein, 8 aa, Lama glama
SSRSTGNT

SEQ ID NO: 263 - protein, 7 aa, Lama glama
VDWKGDT

SEQ ID NO: 264 - protein, 8 aa, Lama glama
FSRSTGNT

SEQ ID NO: 265 - protein, 7 aa, Lama glama
IDNGGRP

SEQ ID NO: 266 - protein, 8 aa, Lama glama
IASSDGST

SEQ ID NO: 267 - protein, 7 aa, Lama glama
SRGGGNA

SEQ ID NO: 268 - protein, 8 aa, Lama glama
IDAGGGST

SEQ ID NO: 269 - protein, 8 aa, Lama glama
IDASGGRT

SEQ ID NO: 270 - protein, 8 aa, Lama glama
SSRSTGNT

SEQ ID NO: 271 - protein, 8 aa, Lama glama
SSRSTGNT

SEQ ID NO: 272 - protein, 8 aa, Lama glama
IDAGGTST

SEQ ID NO: 273 - protein, 8 aa, Lama glama
IATSDGST

SEQ ID NO: 274 - protein, 7 aa, Lama glama
INNGGDV

SEQ ID NO: 275 - protein, 8 aa, Lama glama
SSRSTGNT

SEQ ID NO: 276 - protein, 7 aa, Lama glama
IDWKGDT

SEQ ID NO: 277 - protein, 7 aa, Lama glama
INNGGDA

SEQ ID NO: 278 - protein, 8 aa, Lama glama
SSRSTGNT

SEQ ID NO: 279 - protein, 8 aa, Lama glama
SSRSTGNT

SEQ ID NO: 280 - protein, 7 aa, Lama glama
ISSGGIT

SEQ ID NO: 281 - protein, 7 aa, Lama glama
ISSGGIT

SEQ ID NO: 282 - protein, 8 aa, Lama glama
SSRSTGNT

SEQ ID NO: 283 - protein, 7 aa, Lama glama
IDNGGST

SEQ ID NO: 284 - protein, 7 aa, Lama glama
IDSRGDT

SEQ ID NO: 285 - protein, 6 aa, Lama glama
ISGGNT

SEQ ID NO: 286 - protein, 7 aa, Lama glama
ISSDGST

SEQ ID NO: 287 - protein, 7 aa, Lama glama
ISSSGST

SEQ ID NO: 288 - protein, 8 aa, Lama glama
SSRSTGNT

SEQ ID NO: 289 - protein, 7 aa, Lama glama
LSSDGST

SEQ ID NO: 290 - protein, 8 aa, Lama glama
IDASGGST

SEQ ID NO: 291 - protein, 7 aa, Lama glama
ISIGGST

SEQ ID NO: 292 - protein, 7 aa, Lama glama
IANDGST

SEQ ID NO: 293 - protein, 8 aa, Lama glama
IASSDGST

SEQ ID NO: 294 - protein, 7 aa, Lama glama
IDSRGDT

SEQ ID NO: 295 - protein, 7 aa, Lama glama
IDSSGGT

SEQ ID NO: 296 - protein, 8 aa, Lama glama
FSRSTGNT

SEQ ID NO: 297 - protein, 8 aa, Lama glama
IASSDGST

SEQ ID NO: 298 - protein, 8 aa, Lama glama
LGRNTGNT

SEQ ID NO: 299 - protein, 8 aa, Lama glama
SSRSTGNT

SEQ ID NO: 300 - protein, 7 aa, Lama glama
ISSDGST

SEQ ID NO: 301 - protein, 8 aa, Lama glama
FSRSTGNT

SEQ ID NO: 302 - protein, 7 aa, Lama glama
ISSSGTT

SEQ ID NO: 303 - protein, 7 aa, Lama glama
ITTSGNT

SEQ ID NO: 304 - protein, 7 aa, Lama glama
MTSDGRT

SEQ ID NO: 305 - protein, 8 aa, Lama glama
IDAGGTST

SEQ ID NO: 306 - protein, 7 aa, Lama glama
MTSDGDT

SEQ ID NO: 307 - protein, 7 aa, Lama glama
IGRSGTP

SEQ ID NO: 308 - protein, 7 aa, Lama glama
IDNGGST

SEQ ID NO: 309 - protein, 8 aa, Lama glama
IATSDGST

SEQ ID NO: 310 - protein, 7 aa, Lama glama
INSGGDT

SEQ ID NO: 311 - protein, 8 aa, Lama glama
ASRSTGTT

SEQ ID NO: 312 - protein, 7 aa, Lama glama
ITKSGST

SEQ ID NO: 313 - protein, 10 aa, Lama glama
VAFIGRSGTT

SEQ ID NO: 314 - protein, 7 aa, Lama glama
IGRSGTA

SEQ ID NO: 315 - protein, 7 aa, Lama glama
ITTSGST

SEQ ID NO: 316 - protein, 7 aa, Lama glama
IGRSGTP

SEQ ID NO: 317 - protein, 19 aa, Lama glama
AAARGIYGEISRYTGDYDS

SEQ ID NO: 318 - protein, 15 aa, Lama glama
ATRASINYNYANWPY

SEQ ID NO: 319 - protein, 15 aa, Lama glama
FVIAIPDSAPRPRAY

SEQ ID NO: 320 - protein, 8 aa, Lama glama
ARSAGGLF

SEQ ID NO: 321 - protein, 8 aa, Lama glama
FYNGHLNK

SEQ ID NO: 322 - protein, 8 aa, Lama glama
ARSEGGLF

SEQ ID NO: 323 - protein, 15 aa, Lama glama
AARASINYNYANWPY

SEQ ID NO: 324 - protein, 15 aa, Lama glama
ARGGLLLPKPELFEY

SEQ ID NO: 325 - protein, 15 aa, Lama glama
AARGSPGYVASLYAY

SEQ ID NO: 326 - protein, 9 aa, Lama glama
NARTKATAK

SEQ ID NO: 327 - protein, 15 aa, Lama glama
ATRASINYNYANWPY

SEQ ID NO: 328 - protein, 15 aa, Lama glama
AARASINYNYANWPY

SEQ ID NO: 329 - protein, 15 aa, Lama glama
ATRASINYNYANWPY

SEQ ID NO: 330 - protein, 19 aa, Lama glama
AAARGIYGEISRYTGDYES

SEQ ID NO: 331 - protein, 8 aa, Lama glama
ARSEGGLF

SEQ ID NO: 332 - protein, 8 aa, Lama glama
FYNGHVNK

SEQ ID NO: 333 - protein, 15 aa, Lama glama
ARGGSLVLRPKYYDY

SEQ ID NO: 334 - protein, 15 aa, Lama glama
AARASINYNYANWPY

SEQ ID NO: 335 - protein, 15 aa, Lama glama
AARASINYNYANWPY

SEQ ID NO: 336 - protein, 15 aa, Lama glama
FVVGISDSTPRPRAY

SEQ ID NO: 337 - protein, 15 aa, Lama glama
FVIAIPDSAPRPRAY

SEQ ID NO: 338 - protein, 15 aa, Lama glama
FVIAIPDSAPRPRAY

SEQ ID NO: 339 - protein, 15 aa, Lama glama
ARGGLLLPKPELFDD

SEQ ID NO: 340 - protein, 15 aa, Lama glama
FVVGISDSMPRPRAY

SEQ ID NO: 341 - protein, 10 aa, Lama glama
ARSEGGLFRG

SEQ ID NO: 342 - protein, 15 aa, Lama glama
AARASASYVARMYAY

SEQ ID NO: 343 - protein, 15 aa, Lama glama
AARASAAHVARLYPY

SEQ ID NO: 344 - protein, 9 aa, Lama glama
RIVLPSRII

SEQ ID NO: 345 - protein, 15 aa, Lama glama
AARASINYNYANWPY

SEQ ID NO: 346 - protein, 17 aa, Lama glama
NAEVKRHYYYDSAGHRY

SEQ ID NO: 347 - protein, 15 aa, Lama glama
AARGSPGYVAKLYAH

SEQ ID NO: 348 - protein, 15 aa, Lama glama
AARASTSYVASRYPY

SEQ ID NO: 349 - protein, 10 aa, Lama glama
WTAIAGQGEY

SEQ ID NO: 350 - protein, 8 aa, Lama glama
FYNGHLNK

SEQ ID NO: 351 - protein, 5 aa, Lama glama
NLGDY

SEQ ID NO: 352 - protein, 8 aa, Lama glama
FYNGYLNK

SEQ ID NO: 353 - protein, 15 aa, Lama glama
AATVLDIPTMSTDDY

SEQ ID NO: 354 - protein, 9 aa, Lama glama
SARTIATVK

SEQ ID NO: 355 - protein, 8 aa, Lama glama
FYNGHLNK

SEQ ID NO: 356 - protein, 8 aa, Lama glama
FYNAHLNK

SEQ ID NO: 357 - protein, 15 aa, Lama glama
AARGSAAYVARLYPY

SEQ ID NO: 358 - protein, 15 aa, Lama glama
AARASAAHVARLYPY

SEQ ID NO: 359 - protein, 15 aa, Lama glama
AARASINYNYANWPY

SEQ ID NO: 360 - protein, 15 aa, Lama glama
ATRASINYNYANWPY

SEQ ID NO: 361 - protein, 5 aa, Lama glama
NLGDY

SEQ ID NO: 362 - protein, 11 aa, Lama glama
AAFTPEGGWNY

SEQ ID NO: 363 - protein, 15 aa, Lama glama
ATRASINYNYANWPY

SEQ ID NO: 364 - protein, 15 aa, Lama glama
AVRASPGYVASLYPY

SEQ ID NO: 365 - protein, 15 aa, Lama glama
AARASINYNYANWPY

SEQ ID NO: 366 - protein, 8 aa, Lama glama
VRSAGGQF

SEQ ID NO: 367 - protein, 12 aa, Lama glama
NAERIWPNRRSF

SEQ ID NO: 368 - protein, 8 aa, Lama glama
ARSEGGQF

SEQ ID NO: 369 - protein, 15 aa, Lama glama
ATRASINYNYANWPY

SEQ ID NO: 370 - protein, 12 aa, Lama glama
AADDQGDNWYDH

SEQ ID NO: 371 - protein, 15 aa, Lama glama
ATRASINYNYANWPY

SEQ ID NO: 372 - protein, 15 aa, Lama glama
FVVGKSDSMPRARAY

SEQ ID NO: 373 - protein, 15 aa, Lama glama
AARASINYNYANWPY

SEQ ID NO: 374 - protein, 9 aa, Lama glama
RSTVGNVLY

SEQ ID NO: 375 - protein, 15 aa, Lama glama
ARGGLLLPKPELFDS

SEQ ID NO: 376 - protein, 15 aa, Lama glama
ARGGLLLPKPELFDF

SEQ ID NO: 377 - protein, 17 aa, Lama glama
NAEVKRHYYYDSAGYRY

SEQ ID NO: 378 - protein, 8 aa, Lama glama
FYNGHLNK

SEQ ID NO: 379 - protein, 15 aa, Lama glama
ATRASINYNYANWPY

SEQ ID NO: 380 - protein, 15 aa, Lama glama
ATRASINYNYANWPY

SEQ ID NO: 381 - protein, 23 aa, Lama glama
AADLMGVHRRCSGYLNLEYGMDY

SEQ ID NO: 382 - protein, 15 aa, Lama glama
ATRASINYNYANWPY

SEQ ID NO: 383 - protein, 8 aa, Lama glama
FYNGHLNK

SEQ ID NO: 384 - protein, 15 aa, Lama glama
FVVGISDSTPRPRAY

SEQ ID NO: 385 - protein, 15 aa, Lama glama
AARASPGYVGSLYPY

SEQ ID NO: 386 - protein, 12 aa, Lama glama
YANPFLGSWSNY

SEQ ID NO: 387 - protein, 9 aa, Lama glama
RSTVGNVLY

SEQ ID NO: 388 - protein, 9 aa, Lama glama
NGRTKATVK

SEQ ID NO: 389 - protein, 8 aa, Lama glama
FYNGHLNK

SEQ ID NO: 390 - protein, 14 aa, Lama glama
HAKKVEPTLFTTTY

SEQ ID NO: 391 - protein, 19 aa, Lama glama
ALGRRENYYALLQQRGYDY

SEQ ID NO: 392 - protein, 8 aa, Lama glama
FYNGHLNK

SEQ ID NO: 393 - protein, 12 aa, Lama glama
YANPFLGSWSNY

SEQ ID NO: 394 - protein, 15 aa, Lama glama
AARGSPGYVARLYAH

SEQ ID NO: 395 - protein, 8 aa, Lama glama
ARSAGGQF

SEQ ID NO: 396 - protein, 14 aa, Lama glama
NAKKVEPTLFTTTY

SEQ ID NO: 397 - protein, 8 aa, Lama glama
FWNGYTNK

SEQ ID NO: 398 - protein, 11 aa, Lama glama
NAATRLSSTNY

SEQ ID NO: 399 - protein, 12 aa, Lama glama
NAERIWPNRRSF

SEQ ID NO: 400 - protein, 10 aa, Lama glama
NVRWTMGGNY

SEQ ID NO: 401 - protein, 8 aa, Lama glama
FYNGYLNK

SEQ ID NO: 402 - protein, 8 aa, Lama glama
FYNGHLNK

SEQ ID NO: 403 - protein, 15 aa, Lama glama
ATRASINYNYANWPY

SEQ ID NO: 404 - protein, 12 aa, Lama glama
NAERIWPNRRTF

SEQ ID NO: 405 - protein, 8 aa, Lama glama
ARSEGGLF

SEQ ID NO: 406 - protein, 9 aa, Lama glama
RSTVGNLLY

SEQ ID NO: 407 - protein, 15 aa, Lama glama
AARASINYNAANWPY

SEQ ID NO: 408 - protein, 15 aa, Lama glama
ARGGLLLPRPELYEY

SEQ ID NO: 409 - protein, 15 aa, Lama glama
AARASINYNYASWPY

SEQ ID NO: 410 - protein, 15 aa, Lama glama
FVIGMSDSAPRARAY

SEQ ID NO: 411 - protein, 15 aa, Lama glama
ATRASINYNAATWPY

SEQ ID NO: 412 - protein, 8 aa, Lama glama
ARSAGGQF

SEQ ID NO: 413 - protein, 15 aa, Lama glama
AARASINYNYANWPY

SEQ ID NO: 414 - protein, 15 aa, Lama glama
KAAARVTEYSYEYDY

SEQ ID NO: 415 - protein, 10 aa, Lama glama
WTAIAGQGEY

SEQ ID NO: 416 - protein, 15 aa, Lama glama
AARASINYNYANWPY

SEQ ID NO: 417 - protein, 17 aa, Lama glama
KAHISRSYYYDSLGYDY

SEQ ID NO: 418 - protein, 15 aa, Lama glama
AARGSPAYVARLYAY

SEQ ID NO: 419 - protein, 14 aa, Lama glama
TRASVNYNYAAWPY

SEQ ID NO: 420 - protein, 15 aa, Lama glama
AARASINYNYANWPY

SEQ ID NO: 421 - protein, 15 aa, Lama glama
AARGSPGYVARLYAH

SEQ ID NO: 422 - protein, 15 aa, Lama glama
AARASINYNYANWPY

SEQ ID NO: 423 - protein, 15 aa, Lama glama
FVIAIPDSAPRPRAY

SEQ ID NO: 424 - protein, 15 aa, Lama glama
ARGGLLLPKPELFDS

SEQ ID NO: 425 - protein, 9 aa, Lama glama
IAKTKATTK

SEQ ID NO: 426 - protein, 8 aa, Lama glama
ARSEGGLF

SEQ ID NO: 427 - protein, 8 aa, Lama glama
ARSAGGES

SEQ ID NO: 428 - protein, 15 aa, Lama glama
AARASINYNYANWPY

SEQ ID NO: 429 - protein, 15 aa, Lama glama
ATRASINYNYANWPY

SEQ ID NO: 430 - protein, 8 aa, Lama glama
ARSEGGLF

SEQ ID NO: 431 - protein, 15 aa, Lama glama
ARGGLLLPKPELFDG

SEQ ID NO: 432 - protein, 12 aa, Lama glama
SARTPSLRVTWF

SEQ ID NO: 433 - protein, 15 aa, Lama glama
ATRASINYNYAAWPY

SEQ ID NO: 434 - protein, 15 aa, Lama glama
AARGSPGYVARLYAH

SEQ ID NO: 435 - protein, 14 aa, Lama glama
NAKKVKPSLFTNTY

SEQ ID NO: 436 - protein, 15 aa, Lama glama
ATRASINYNYANWPY

SEQ ID NO: 437 - protein, 15 aa, Lama glama
AARASINYNYANWPY

SEQ ID NO: 438 - protein, 8 aa, Lama glama
FYNGYLNK

SEQ ID NO: 439 - protein, 8 aa, Lama glama
FYNGHLNK

SEQ ID NO: 440 - protein, 15 aa, Lama glama
ATRASINYNYANWPY

SEQ ID NO: 441 - protein, 15 aa, Lama glama
FVIAIPDSAPRPRSY

SEQ ID NO: 442 - protein, 15 aa, Lama glama
AARASPAYVARLYAY

SEQ ID NO: 443 - protein, 9 aa, Lama glama
NGRTKATVK

SEQ ID NO: 444 - protein, 8 aa, Lama glama
FYNGHVNK

SEQ ID NO: 445 - protein, 17 aa, Lama glama
NAHISRSYYYDSEGYRY

SEQ ID NO: 446 - protein, 15 aa, Lama glama
ATRASVNYNYAAWPY

SEQ ID NO: 447 - protein, 8 aa, Lama glama
FYNGHLNK

SEQ ID NO: 448 - protein, 8 aa, Lama glama
ARSEGGQF

SEQ ID NO: 449 - protein, 12 aa, Lama glama
NAERIWPNRRTF

SEQ ID NO: 450 - protein, 15 aa, Lama glama
FVVGISDSTPRPRAY

SEQ ID NO: 451 - protein, 15 aa, Lama glama
ARGGSLVLRPGHYDY

SEQ ID NO: 452 - protein, 15 aa, Lama glama
AARGSASYVARLYPY

SEQ ID NO: 453 - protein, 15 aa, Lama glama
AARGAISYVARLYAY

SEQ ID NO: 454 - protein, 15 aa, Lama glama
AARASINYNYANWPY

SEQ ID NO: 455 - protein, 15 aa, Lama glama
ARGGLLLPKPELFEY

SEQ ID NO: 456 - protein, 15 aa, Lama glama
AARASINYNAANWPY

SEQ ID NO: 457 - protein, 15 aa, Lama glama
AARASINYNYANWPY

SEQ ID NO: 458 - protein, 19 aa, Lama glama
FWNGHTNKWGQGTQVTVSS

SEQ ID NO: 459 - protein, 15 aa, Lama glama
AARASINYNYANWPY

SEQ ID NO: 460 - protein, 9 aa, Lama glama
NAILPSRSI

SEQ ID NO: 461 - protein, 10 aa, Lama glama
WTAIAGQGEY

SEQ ID NO: 462 - protein, 12 aa, Lama glama
YANPFLGSWSNY

SEQ ID NO: 463 - protein, 8 aa, Lama glama
ARSEGGLF

SEQ ID NO: 464 - protein, 12 aa, Lama glama
YANPFLGSWSNY

SEQ ID NO: 465 - protein, 5 aa, Lama glama
HLGDT

SEQ ID NO: 466 - protein, 15 aa, Lama glama
FVIAIPDSAPRPRAY

SEQ ID NO: 467 - protein, 15 aa, Lama glama
ARGGLLLPRPELYEY

SEQ ID NO: 468 - protein, 20 aa, Lama glama
YTAIGWRNYWGQGTQVTVSS

SEQ ID NO: 469 - protein, 15 aa, Lama glama
AARASINYNYANWPY

SEQ ID NO: 470 - protein, 10 aa, Lama glama
WTAIAGQGEY

SEQ ID NO: 471 - protein, 5 aa, Lama glama
NLGDY

SEQ ID NO: 472 - protein, 5 aa, Lama glama
NLGDY

SEQ ID NO: 473 - protein, 10 aa, Lama glama
WTAIAGQGEY

SEQ ID NO: 474 - protein, 5 aa, Lama glama
HLGDT

SEQ ID NO: 475 - protein, 126 aa, Lama glama
QVQLVESGGGLVQPGGSLRLSCAASGRTFSNYAMGWFRQAPGKERRFVAGISRDDTSTSYADSVKGRFTILRDRSKNTVDLQMNSLKPEDTAIYYCAAARGIYGEISRYTGDYDSWGQGTQVTVSS

SEQ ID NO: 476 - protein, 122 aa, Lama glama
QLQLVESGGGLVQPGGSLRLSCAASGRIFGSYVMGWFRQAPGKEHEFVAASSRSTGNTVYLDSVKGRFTISRDDAKNTLYLQMNSLKPEDTAVYYCATRASINYNYANWPYRGQGTQVTVSS

SEQ ID NO: 477 - protein, 121 aa, Lama glama
QLQLVESGGGLVQSGGSLRLSCTASGSTVRVTAGGWYRQAPGNERELVADIDNGGRPTYADSVKGRFTISRDNAKNAVDLQMNNLKPEDTAVYYCFVIAIPDSAPRPRAYWGQGTQVTVSS

SEQ ID NO: 478 - protein, 115 aa, Lama glama
QVQLVESGGGLVQPGGSLRLSCAASGFTFSMKWMYWVRQGPGKGLEWVSTIDTSGSSTYYADSVKGRFTISRDNSKNTVYLQMNSLESADTAVYYCARSAGGLFRGRGTQVTVSS

SEQ ID NO: 479 - protein, 114 aa, Lama glama
QLQLVESGGGLVQAGGSLRLSCAASRTTFSFNYMGWYRQAPGNQRDLVARISSGGITNYADSVKGRFTISGDNAKNTVYLQMNSLKPEDTARYYCFYNGHLNKWGQGTQVTVSS

SEQ ID NO: 480 - protein, 115 aa, Lama glama
QLQLVESGGGLVQPGGSLRLSCAASGFTFSTKWMYWVRQGPGKGLERVAAIDASGGSTYYADSVKGRFTISRDNSENTLYLQMNSLESADTAVYYCARSEGGLFRGRGTQVTVSA

SEQ ID NO: 481 - protein, 122 aa, Lama glama
QVQLVESGGGLVQAGGSLRLSCAASGRTVGSYVMGWFRQAPGKERELLAASSRSTGNTVYADSVKGRFTISRDDAKNTVFLQMNSLKPEDTAVYYCAARASINYNYANWPYWGQGTQVTVSP

SEQ ID NO: 482 - protein, 121 aa, Lama glama
QLQLVESGGGLVQAGGSLRLACAASGFSFDDYAIGWFRQAPGKEREGVSCITSDGSTYYADSVKGRFTISSDNAKNTVYLQMNILKPEDTAVYYCARGGLLLPKPELFEYWGQGTQVTVSS

SEQ ID NO: 483 - protein, 121 aa, Lama glama
QVQLVESGGGLVQAGDSLRLSCAPSGLTASTYAMAWFRQAPGKEREFVSVIDWKGDTTYANSVKGRFTISRDNGKNTVYLQMNSLQPEDTALYYCAARGSPGYVASLYAYWGRGTQVTVSS

SEQ ID NO: 484 - protein, 114 aa, Lama glama
QLQLVESGGGLVQAGGSLRLSCVASGTTFTIEDIGWYRLAPGKQRELVAIISGGNTNYADSVKGRFTISRDNAKNTVFLQMNSLKPEDTAVYYCNARTKATAKWGRGIQVTVSS

SEQ ID NO: 485 - protein, 121 aa, Lama glama
QLQLVESGGGLVQAGGSLRLSCAASGRIFGTYVMGWFRQAPGKEREFVAAVSRSTGSTYADSVKGRFTISRDDAKNMLYLQMNNLRPEDTAIYYCATRASINYNYANWPYWGQGTQVTVSS

SEQ ID NO: 486 - protein, 122 aa, Lama glama
QVQLVESGGGLVQPGGSLRLSCAASGRTFGSYVMGWFRQAPGKEREFVAAASRSTGNTVYPDSVKGRFTISRDDAKNTLYLQMNSLKPEDTAVYYCAARASINYNYANWPYWGQGTQVTVSS

SEQ ID NO: 487 - protein, 122 aa, Lama glama
QVQLVESGGGLVQAGGSLRLSCAASGRTFGTYVMGWFRQAPGKEREFVAAFSRSTGSTTYADSVKGRFTISRDDAKNTLYLQMNRLKPEDTAVYYCATRASINYNYANWPYWGQGTQVTVSS

SEQ ID NO: 488 - protein, 126 aa, Lama glama
QVQLVESGGRLVQAGGSLRLSCAASGRTSSNYAMGWFRQAPGKERRFVAGISRDGADTSYADSVKGRFTILRDRSKNTADLQMNSLKPEDTAIYYCAAARGIYGEISRYTGDYESWGQGTQVTVSS

SEQ ID NO: 489 - protein, 115 aa, Lama glama
QLQLVESGGGLVQPGGSLRLSCAASGFTFSTKWMYWVRQGPGKGLERVAAIDASGGSTYYADSVKGRFTISRDNSENTVYLQMNSLESADTAVYYCARSEGGLFRGRGTQVTVSA

SEQ ID NO: 490 - protein, 114 aa, Lama glama
QLQLVESGGGLVQAGGSLRLSCAASGTTSSFVYMGWYRQAPGNQRDLVARISSDGSTNYADSVKGRFTISGDNAKNTVYLQMNSLKPEDTARYYCFYNGHVNKWGQGTQVTVSS

SEQ ID NO: 491 - protein, 122 aa, Lama glama
QLQLVESGGGLVQPGGSLRLSCAASGFTLDGYAIGWFRQAPGKEREGVSSIATSDGSTYYADSVKGRFTISGDDAKNTVYLHMNSLKPEDTAVYYCARGGSLVLRPKYYDYWGQGTQVTVSS

SEQ ID NO: 492 - protein, 122 aa, Lama glama
QLQLVESGGGLVQPGGSLRLSCAASGRTVGSYVMGWFRQAPGKERELLAASSRSTGNTVYADSVKGRFTISRDDAKNTLYLQMNSLKPEDTAVYYCAARASINYNYANWPYWGQGTQVTVSS

SEQ ID NO: 493 - protein, 122 aa, Lama glama
QVQLVESGGGLVQAGGSLRLSCAASGRTVGSYAMGWFRQAPGKEREFLAAFSRSTGNTVYADSVKGRFTISRDDAKNTLYLQMNSLKPEDTAVYYCAARASINYNYANWPYWGQGTQVTVSS

SEQ ID NO: 494 - protein, 121 aa, Lama glama
QVQLVESGGGLVQAGGSLRLSCTTSGSTFSITAGGWYRQAPGEQRELVADVANDGSTSYAESVKGRFTISRDNAKNIVELQMSSLELEDTAVYVCFVVGISDSTPRPRAYWGQGTQVTVAS

SEQ ID NO: 495 - protein, 121 aa, Lama glama
QLQLVESGGGLVQAGGSMRLSCTASGSTVRVTAGGWYRQAPGNERELVADIDNGGSTTYADSVKGRFTISRDNAKNAVDLQMNSLKPEDTAVYRCFVIAIPDSAPRPRAYWGQGTQVTVSS

SEQ ID NO: 496 - protein, 121 aa, Lama glama
QLQLVQSGGGLVQAGGSLRLSCTASGSTVRVTAGGWYRQTPGNERELVADIDNGGSTTYADSVKGRFTISRDNAKNAVDLQMNSLKPEDTAVYRCFVIAIPDSAPRPRAYWGQGTEVTVSS

SEQ ID NO: 497 - protein, 121 aa, Lama glama
QLQLVESGGGLVQPGGSLRLACAASGFSFDDYAIGWFRQAPGKEREGVSCITSDGSTYYADSVKGRFTISSDYAKNTVHLQMNSLKPEDTAVYYCARGGLLLPKPELFDDWGQGTQVTVSS

SEQ ID NO: 498 - protein, 121 aa, Lama glama
EVQLVESGGGLVQAGGSLRVSCTTSGSTFSITAGGWYRQTPGKERELVADIANDGSTTYANSVKGRFTISRDNAKNTVELQMSSLKLEDTAAYRCFVVGISDSMPRPRAYWGQGTQVTVSS

SEQ ID NO: 499 - protein, 115 aa, Lama glama
QLQLVESGGGLVQPGGSLRLSCAASGFTFSTKWMYWVRQGPGKGLERVAAIDAGGGSTYYADSVKGRFTISRDNSENTVYLQMNSLESADTAVYYCARSEGGLFRGRGTQVTVSA

SEQ ID NO: 500 - protein, 121 aa, Lama glama
QVQLVESGGGVVQAGGSLRLSCAASGLTSSTYVMAWFRQAPGKEREFVSVIDSRGDTTYAKSVKGRFSISRDNGKNAVYLQMNSLQPEDTALYYCAARASASYVARMYAYWGRGTQVTVSS

SEQ ID NO: 501 - protein, 121 aa, Lama glama
EVQLVESGGGLVQAGGSLRLSCAASGLTHSTYVMAWFRQAPGKEREFVSVIDSSGDTTYANSVKGRFTISRDNGKMTVYLQMNSLQPEDTALYYCAARASAAHVARLYPYWGRGTQVTVSS

SEQ ID NO: 502 - protein, 115 aa, Lama glama
QLQLVESGGGLVQAGGSLRLSCAASRTTFRINGVDWYRQVPGKQRELVATASSDGPTNYAESVKGRFTISRDNAKNTVYLQMNSLKPEDTAVYYCRIVLPSRIIWGQGTQVTVSS

SEQ ID NO: 503 - protein, 122 aa, Lama glama
QLQLVESGGGLVQAGGSLRLSCVASGRTFSTYAMGWFRQAPGKEREFVGAIRWDGGSTNYANSVKGRFTISRDNAKNTVYLQVNSLKPEDTAVYYCAARASINYNYANWPYWGRGTQVTVSS

SEQ ID NO: 504 - protein, 123 aa, Lama glama
QVQLVESGGGLVQPGGSLTLSCAASGTIESINDMGWYRQAPGKQRELVAAITSGGSTNYADSVKGRFTISRDNAKNTVYLQMNSLKPEDTAVYYCNAEVKRHYYYDSAGHRYWGQGTQVTVSS

SEQ ID NO: 505 - protein, 121 aa, Lama glama
QLQLVESGGGLVQAGDSLRLSCALSGLAASTYAMAWFRQAPGKEREFVSVIDWKGDSTTANSVKGRFTISRDHGKNTVYLQMNSLQPEDTALYYCAARGSPGYVAKLYAHWGRGTQVTVSS

SEQ ID NO: 506 - protein, 121 aa, Lama glama
QLQLVESGGGLVQAGGSLRLSCAASGLTSSTYVMAWFRQAPGKEREFVSSVDWSGDRTYSNSVKDRFTISRDNGKNTVYLQMNSLQPEDTALYYCAARASTSYVASRYPYWGRGTQVTVSS

SEQ ID NO: 507 - protein, 116 aa, Lama glama
QLQLVESGGGLVQPGGSLRLSCVLSGSTFGRNVITWYRQPPGKQREFIAMITTSGSTRYADSVKGRFTMSRDTAKNTAFLQMSNLKLEDTAVYYCWTAIAGQGEYWGQGTQVTVSS

SEQ ID NO: 508 - protein, 114 aa, Lama glama
QLQLVESGGGLVQAGGSLRLSCAASGTTFSFVYMGWYRQAPGNQRDLVARISSDGSTNYADSVKGRFTISRDNAKNTVYLQMNSLRPEDTARYYCFYNGHLNKWGQGTQVTVSS

SEQ ID NO: 509 - protein, 115 aa, Lama glama
QVQLVESGGGLVQAGGSLRLSCAASGSTFSINVMAWYRQAPGEQRELERELVAFIGRTGTTKYGDSVKGRFTISRDNAKNTVYLQMDSLRPEDTAVYYCNLGDYWGQGTQVTVSS

SEQ ID NO: 510 - protein, 114 aa, Lama glama
QLQLVESGGGLVQAGGSLRLSCAASGSTFSFNYMGWYRQAPGNQRDLVARISSGGITNYADSVKGRFTISGDNAKDTVYLQMNSLKPEDTARYYCFYNGYLNKWGQGTQVTVSS

SEQ ID NO: 511 - protein, 121 aa, Lama glama
EVQLVESGGGLVQAGESLRLSCVVSGSRFRISGMGWYRQAPEKQRELVAAITRGGSTNYSDSVKGRFTISSDNAKNTVYLQMNSLKPDDTDVYYCAATVLDIPTMSTDDYWGQGTQVTVSS

SEQ ID NO: 512 - protein, 115 aa, Lama glama
QLQLVESGGGLVQAGGSLRLSCVASGTTFSIEETYWYRLAPGKQRELVAVISGGGNTNYADSVKGRFTISRDNAKNTVYLQMNSLKPEDTAVYYCSARTIATVKWGQGIQVTVSS

SEQ ID NO: 513 - protein, 114 aa, Lama glama
QVQLVESGGGLVQAGGSLRLSCAASGTTFSFNYMGWYRQAPGNQRDLVARISSGGITNYADSVKGRFTISGDNAKNVVYLQMNSLKPEDTARYYCFYNGHLNKWGQGTQVTVSS

SEQ ID NO: 514 - protein, 114 aa, Lama glama
QVQLVESGGGLVQAGGSLRLSCAASGTTFSFVYMGWFRQAPGNQRDLVARISSDGSTNYADSVKGRFTISGDNAKNTVYLQMNSLKPEDTARYFCFYNAHLNKWGQGTQVTVSS

SEQ ID NO: 515 - protein, 121 aa, Lama glama
QVQLVESGGGAVQAGGSLRLSCAASGLTSSTYVMAWFRQAPGKEREFVSVIDSRGDTTYANSVKGRFTISRDNGKNTVYLQMNSLQPTDTALYSCAARGSAAYVARLYPYWGRGTQVTVSS

SEQ ID NO: 516 - protein, 121 aa, Lama glama
QVQLVESGGGLVQAGGSLRLSCVASGLAHSTYVMAWFRQAPGKEREFVSVIDWSGDTTYANSVKGRFTISRDNGKNTVYLQMNSLQPEDTALYYCAARASAAHVARLYPYWGRGTQVTVSS

SEQ ID NO: 517 - protein, 122 aa, Lama glama
QLQLVESGGGLVQAGGSLRLSCAASGRTFGTYVIGWFRQAPGKEREFVAAFSRSTGNTVYPDSVKGRFTISRDDAKNTVTLQMNSLKPEDTAVYYCAARASINYNYANWPYWGQGTQVTVSS

SEQ ID NO: 518 - protein, 122 aa, Lama glama
QLQLVESGGGLVQPGGSLRLSCAASGRTVGSYVMGWFRQAPGKEREFLAANSRSTGNTVYADSVKGRFTISRDDAKNTLYLQMNSLKPEDTAVYYCATRASINYNYANWPYWGQGTQVTVSS

SEQ ID NO: 519 - protein, 114 aa, Lama glama
QLQLVESGGGLVQAGGSLRLSCAASGSTFPINVFAWYRQAPGEQRELGELVAFIGRSGTTNYGDSVKGRFTISRDNAKNTVYLQMDSLRPEDTAVYYCNLGDYWGQGTQVTVSS

SEQ ID NO: 520 - protein, 118 aa, Lama glama
QLQLVESGGGLVKAGGSLRLSCAASGRTFSSYRMGWFRQAPGKERESVAHITGDGNSIYYADSAKGRFTISVDYAKNTVYLQMNSLKFEDTATYYCAAFTPEGGWNYWGQGTQVTVSS

SEQ ID NO: 521 - protein, 122 aa, Lama glama
QLQLVESGGGLVQAGGSLRLSCAASGRTVGSYVMGWFRQAPGKEREFLAASSRSTGNTVYADSVKGRFSISRDDAKNTLYLQMNSLKPEDTAVYYCATRASINYNYANWPYWGQGTQVTVSS

SEQ ID NO: 522 - protein, 121 aa, Lama glama
EVQLVESGGGLVQAGGSLRLSCAASGLTASTYVMAWFRQAPGKERDFVSLIDWRGEPTYANSVKGRFTISRDNGKNTVFLQMNSLRPEDTALYYCAVRASPGYVASLYPYWGRGTQVTVSS

SEQ ID NO: 523 - protein, 122 aa, Lama glama
QLQLVESGGGLVQAGGSLRLSCVASGRTVGSYAVAWFRQAPGKERELLAASSRSTGNTVYADSVKGRFTIARDDAKNTLYLQMNSLKPEDTAVYYCAARASINYNYANWPYWGQGTQVTVSS

SEQ ID NO: 524 - protein, 115 aa, Lama glama
EVQLVESGGGLVQPGGSLRLSCAASGFTFSTKWMYWVRQGPGKGLEWVSTIDASGGSTYYADSVKGRFTISRDNTEKKVYLQMNSLESADTAVYYCVRSAGGQFRGRGTQVTVSS

SEQ ID NO: 525 - protein, 118 aa, Lama glama
QLQLVESGGGLVQAGGSLRLSCAASGTTFDMNSMGWYRQAPGFKRDLVATISIGGDTNYTDSVKGRFTISRDNAKNTVYMQMNSLKPEDTAVYYCNAERIWPNRRSFWGQGTQVTVSS

SEQ ID NO: 526 - protein, 115 aa, Lama glama
QVQLVESGGGLVQPGGSLRLSCKASGFTFSTKWMYWVRQGPGKGLERVAAIDASGGSTYYADSVKGRFTISRDNSDNTVYLQMNSLESADTAVYYCARSEGGQFRGQGTQVTVSS

SEQ ID NO: 527 - protein, 122 aa, Lama glama
QVQLVESGGGLVQAGGSLRLSCAASGRTFGTYVMGWFRQAPGKEREFVAAFSRSTGSTTYADSVKGRFTISRDDAKNTLYLQMNSLKPEDTAVYYCATRASINYNYANWPYWGQGTQVTVSS

SEQ ID NO: 528 - protein, 119 aa, Lama glama
QVQLVESGGGLVQAGGSLRLSCAASGRPISAYTWAWFRQAPGKEREFVAAITSSAGTTYYADSVRGRFTISRDNAENTVYLRMNTLKPEDTAVYYCAADDQGDNWYDHWGQGTQVTVSS

SEQ ID NO: 529 - protein, 122 aa, Lama glama
QVQLVESGGGLVQAGGSLRLSCAASGRVFGTYVMGWFRQAPGKEREFVAAFSRSTGHITYADSVKGRFTISRDDAKNTLYLQMNSLKPEDTAVYYCATRASINYNYANWPYWGQGTQVTVSS

SEQ ID NO: 530 - protein, 121 aa, Lama glama
QVQLVESGGGLVQAGGSLRLSCTTSGSTFSITAGGWYRQAPGKQRELVADIANDGSTTSADSVKGRFTISRDNAKNTVELQMSSLKLEDTAVYYCFVVGKSDSMPRARAYWGQGTQVTVSS

SEQ ID NO: 531 - protein, 122 aa, Lama glama
QVQLVESGGGLVQAGGSLRLSCAASGRTIGSYVMGWFRQAPGKEREFVVAFSRSTGHISYADSVKGRFTISRDDAKNTLYLQMNSLKPEDTAVYYCAARASINYNYANWPYWGQGTQVTVSS

SEQ ID NO: 532 - protein, 116 aa, Lama glama
QVQLVESGGGLVQAGGSLRLSCAASGRTFSSGALGWFRQAPGKEREFVATIRWSDERKQYSEFVKGRFTISRDSAKNTVYLQMNNLQPEDTAVYYCRSTVGNVLYWGQGTQVTVSS

SEQ ID NO: 533 - protein, 122 aa, Lama glama
QLQLVESGGDLVQAGGSLRLSCAASGFTLDDYAIGWFRQAPGKEREGVSCIASSDGSTYYADSVKGRFTISTDNAKNTVYLQMNSLKPEDTAVYYCARGGLLLPKPELFDSWGQGTQVTVSS

SEQ ID NO: 534 - protein, 122 aa, Lama glama
QLQLVESGGGLVQAGGSLTLSCAASGFTLDDYAIGWFRQAPGKEREGVSCIATSDGSTYYADSVKGRFTISSDNAKNTVYLQMNSLKPEDTAVYYCARGGLLLPKPELFDFRGQGTQVTVSP

SEQ ID NO: 535 - protein, 123 aa, Lama glama
QVQLVESGGGLVQPGGSLTLSCAASGTIESINDMGWYRQAPGKQRELVAAITSGGSTNYADSVKGRFTISRDNAKNTVYLQMNSLKPEDTAVYYCNAEVKRHYYYDSAGYRYWGQGTQVTVSS

SEQ ID NO: 536 - protein, 114 aa, Lama glama
QLQLVESGGGLVQAGGSLRLSCAASGTTFSFVYMGWYRQAPGNQRDLVARISRDGSTNYLDSVKGRFTISGDNAKNTVYLQMNSLKPEDTARYYCFYNGHLNKWGRGTQVTVSS

SEQ ID NO: 537 - protein, 123 aa, Lama glama
QLQLVESGGGLVQAGGSLRLSCAASGRTFVGSYVMGWFRQAPGKAREFVAASSRSTGNTVYLDAVKGRFTVSRDDAKNTLYLQMSSLKPEDTAVYYCATRASINYNYANWPYWGQGTQVTVSS

SEQ ID NO: 538 - protein, 122 aa, Lama glama
QVQLVESGGGLVQAGGSLRLSCAASGRTFGSYVMGWFRQAPGKEREFLAAFSRSTGSTVYADSVKGRFTISKDDAKNTLYLQMNSLKPEDTAVYYCATRASINYNYANWPYWGQGTQVTVSS

SEQ ID NO: 539 - protein, 129 aa, Lama glama
QVQLVESGGGLVQAGGSLRLSCAASGFTFDDYAIGWFRQAPGKEREGIACRSTDGSTYYADSVEGRFTISSDNAKRTVYLQMNSLKPGDTAVYYCAADLMGVHRRCSGYLNLEYGMDYWAKGTLVTVSS

SEQ ID NO: 540 - protein, 122 aa, Lama glama
QLQLVESGGGLVQAGGSLRLSCAASGRTVGSYVMGWFRQAPGKEREFLAANSRSTGNTVYADSVKGRFTISRDDAKNTLYLQMNSLKPEDTAVYYCATRASINYNYANWPYWGQGTQVNVSS

SEQ ID NO: 541 - protein, 114 aa, Lama glama
QLQLVESGGGLVQAGGSLRLSCAASGTTFSFVYMGWYRQAPGNQRDLVARISSDGSTNYADSVKGRFTISGDNAKNTVYLQMNSLRPEDTARYYCFYNGHLNKWGQGTQVTVSS

SEQ ID NO: 542 - protein, 121 aa, Lama glama
EVQLVESGGGLVQAGGSLRLSCTTSGSTFSITAGGWYRQAPGKQRELVADIANDGSTTYADSVKGRFTISRDNAKNTVELQMSSLKLEDTAVYLCFVVGISDSTPRPRAYWGQGTQVTVSS

SEQ ID NO: 543 - protein, 121 aa, Lama glama
EVQLVESGGGLVQAGGSLRLSCAASGLTSSTYAMAWFRQAPGKEREFVSVIDWRGSTTYTNSVKGRFTISRDNGKNTVYLQMNSLQPEDTALYYCAARASPGYVGSLYPYWGRGTQVTVSS

SEQ ID NO: 544 - protein, 118 aa, Lama glama
QLQLVESGGGLMQAGGSLRLSCAASGTTSSLNLMGWYRQAPGKKRESVAHMTSDGRTDYTDSVKGRFTISRDNSKNTVYLQMNNLKPEDTAVYYCYANPFLGSWSNYWGQGTQVTVSS

SEQ ID NO: 545 - protein, 116 aa, Lama glama
QVQLVESGGGLVEAGGSLRLSCAASGRTFSSGALGWFRQAPGKEREFVATIRWSDERKQYSEFVKGRFTISRDSAKNTVYLQMNNLQPEDTAVYYCRSTVGNVLYWGQGTQVTVSS

SEQ ID NO: 546 - protein, 114 aa, Lama glama
QLQLVESGGGLVQAGGSLRLSCVASGTTFTIEDIGWYRLAPGKQRELVAIISGGNTNYADSVKGRFTISRDNTKNTVYLQMNSLKPEDTAVYYCNGRTKATVKWGRGTQVTVSS

SEQ ID NO: 547 - protein, 114 aa, Lama glama
QVQLVESGGGLVQAGGSLRLSCAASGTTFSFVYMGWYRQAPGNQRELVARISSDGSTNYSDSVKGRFLISGANAKNTVYLQMNSLKPEDTARYYCFYNGHLNKWGQGTQVTVSS

SEQ ID NO: 548 - protein, 120 aa, Lama glama
QLQLVESGGGLVQAGGSLRLSCAASGSTFNFRIMGWYRQVPGKQRELVAVINSGGSADYTEDVKGRFTISRDDAKSTMYLQMNSLKPEDTAVYYCHAKKVEPTLFTTTYWGQGTQVTVSS

SEQ ID NO: 549 - protein, 126 aa, Lama glama
QVQLVESGGGLVQAGGSLRLSCAASGRTSSNYNMGWFRQAPGKEREFVAAIPWTGSTTYYADPVKGRFTISRDSTKNTGDLQMNSLKPEDTAVYYCALGRRENYYALLQQRGYDYWGQGTQVTVSS

SEQ ID NO: 550 - protein, 114 aa, Lama glama
QLQLVESGGGLVQAGGSLRLSCAASGTTFSFVYMGWYRQAPGNQRDLVARISSDGSTNYADSVKGRFTISGDNAKNIVYLQMNSLKPEDTARYYCFYNGHLNKWGQGTQVTVSS

SEQ ID NO: 551 - protein, 118 aa, Lama glama
QLQLVESGGGLVQAGGSLRLSCAASGTTSSLNLMGWYRQAPGKKRESVAHMTSDGRTDYVDSVKGRFTISRDNSKNTVYLQMNNLKPEDTAVYICYANPFLGSWSNYWGQGTQVTVSS

SEQ ID NO: 552 - protein, 121 aa, Lama glama
QVQLVESGGGLVQAGGSLRLSCAASGLTASTYAMAWFRQAPGKEREFVSVIDWRGDTTYANSVKGRFTISRDNGKSTVYLQMNSLQPEDTALYYCAARGSPGYVARLYAHWGRGTQVTVSS

SEQ ID NO: 553 - protein, 115 aa, Lama glama
EVQLVESGGGLVQAGGSLRLSCAASGFTFSTKWMYWVRQGPGKGLEWVSTIDASGGSTYYADSVKGRFTISRDNTEKKVYLQMNSLESADTAVYYCARSAGGQFRGRGTQVTVSS

SEQ ID NO: 554 - protein, 120 aa, Lama glama
QLQLVESGGGLVQAGGSLRLSCAASGSTFNFNIMGWYRQVPGKQRELVAVINSGGSADYTEDVKGRFTISRDNAKSIMYLQMNSLKPEDTAVYYCNAKKVEPTLFTTTYWGQGTQVTVSS

SEQ ID NO: 555 - protein, 114 aa, Lama glama
QLQLVESGGGLVQAGGSLRLSCAASGTTFSFNYMGWYRQAPGNQRDLVARISSGGSTNYADSVKGRFTISADNAKNTVYLQMNSLKPEDTARYTCFWNGYTNKWGQGTQVTVSS

SEQ ID NO: 556 - protein, 117 aa, Lama glama
QVQLVESGGGLVQHGGSLRLSCAVSGSIFTTATMAWYRQAPGKQRDWVAVITTGGSTRYADDVKGRFAISRDNAKNTVYLQMNNLKPEDTAVYYCNAATRLSSTNYWGQGTQVSVSS

SEQ ID NO: 557 - protein, 118 aa, Lama glama
QLQLVESGGGLVQAGGSLRLSCAASGTTFDMNSMGWYRQAPGFKRDLVATISIGGDTNYADSVKGRFTISRDNAKNTVYMQMNSLKPEDTAVYYCNAERIWPNRRSFWGQGTQVTVSS

SEQ ID NO: 558 - protein, 116 aa, Lama glama
QLQLVESGGGLVQPGGSLRLSCAASGSIGRLNVMGWFRQAPGKQRELVAVISSDNKKDYADSVQGRFTISRDNSKNTVYLQMNSLKPEDTAVYYCNVRWTMGGNYWGQGTQVTVSS

SEQ ID NO: 559 - protein, 114 aa, Lama glama
QLQLVESGGGLVQAGGSLRLSCAASGSTFSFNYMGWYRQAPGNQRDLVARISSGGITNYADSVKGRFTISGDNAKNTVYLQMNSLKPEDTARYYCFYNGYLNKWGRGTQVTVSS

SEQ ID NO: 560 - protein, 114 aa, Lama glama
QLQLVESGGGLVQTGGSLRLSCAASGTTFSFVYMGWYRQAPGNQRDLVARISSDGSTNYADSVKGRFTISGGNAKNTVYLQMNSLKPEDTARYYCFYNGHLNKWGQGTQVTVSS

SEQ ID NO: 561 - protein, 123 aa, Lama glama
QLQLVESGGGLVQAGGSLRLSCAASGRTFVGTYVMGWFRQAPGKAREFVAASSRSTGNTVYLDSVKGRFTVSRDDAKNTLYLQMNSLKPEDTAVYYCATRASINYNYANWPYWGQGTQVTVSS

SEQ ID NO: 562 - protein, 118 aa, Lama glama
EVQLVESGGGLVQAGGSLRLSCAASGTTFDMNSMGWYRQGPGFKRELVATISMGGDTNYADSVKGRFTISRANAKNTVYMQMNSLKPEDTAVYYCNAERIWPNRRTFWGQGTQVTVSS

SEQ ID NO: 563 - protein, 115 aa, Lama glama
QLQLVESGGGSVQPGESLRLSCAASGFTFSTKWMYWVRQGPGKGLERVAAIDAGGTSTYYADSVKGRFTISRDNSENTVYLQMNSLESADTAVYYCARSEGGLFRGRGTQVTVSA

SEQ ID NO: 564 - protein, 116 aa, Lama glama
QLQLVESGGGLVEAGGSLRLSCAASGRTFSSGALGWFRQAPGKEREFVATIRWSDERTQYSEFVKGRFTISRDNAKNTVYLQMNNLQPEDTAVYYCRSTVGNLLYWGQGTQVTVSS

SEQ ID NO: 565 - protein, 123 aa, Lama glama
EVQLVESGGGLVQAGGSLRLSCAASGRTFVGTYVMGWFRQAPGKAREFVAASSRSTGNTVYLDSVKGRFTVSRDDAKNTLYLQMNSLKPEDTAVYYCAARASINYNAANWPYWGQGTQVTVSS

SEQ ID NO: 566 - protein, 122 aa, Lama glama
QLQLVESGGGLVQPGGSLRLSCAVSGFTLDDYAIGWFRQAPGKEREGVSSIATSDGSTYYADSVKGRFTISSDNAKNTVYLQMNSLKPEDTAVYYCARGGLLLPRPELYEYWGQGTQVTVSS

SEQ ID NO: 567 - protein, 122 aa, Lama glama
QVQLVESGGGLVQAGGSLRLSCAASGRTVGTYVIGWFRQAPGKEREFVAAFSRSTGSTVYPDSVKGRFTISRDDAKNTVYLQMNNLKPEDTAVYYCAARASINYNYASWPYWGQGTQVTVSS

SEQ ID NO: 568 - protein, 121 aa, Lama glama
QVQLVESGGGLVQAGGSLRLSCTVSGSTVRLTAGGWYRQVPGNERELVADIDNGGGTTYADSVKGRFTISRDNAKNTVDLQMNSLKPEDTAVYQCFVIGMSDSAPRARAYWGQGTQVTVSS

SEQ ID NO: 569 - protein, 123 aa, Lama glama
EVQLVESGGGLVQAGGSLRLSCAASGRTFVGSYVMGWFRQAPGKEREFVAASSRSTGNTVYLDSVKGRFTVSRDDAKNTLYLQMNSLKTEDTAVYYCATRASINYNAATWPYWGQGTQVTVSS

SEQ ID NO: 570 - protein, 115 aa, Lama glama
QLQLVESGGGLVQPGGSLRLSCAASGFTFSTKWMYWVRQGPGKGLEWVSTIDASGGSTYYADSVKGRFTISRDNTEKKVYLQMNSLESADTAVYYCARSAGGQFRGRGTQVTVSS

SEQ ID NO: 571 - protein, 122 aa, Lama glama
QLQLVESGGGLVQAGGSLRLSCAASGRTVGSYAMGWFRQAPGKEREFLAAFSRSTGPTVYADSVKGRFTISRDDAKNTLYLQMNSLKPEDTAVYYCAARASINYNYANWPYWGQGTQVTVSS

SEQ ID NO: 572 - protein, 121 aa, Lama glama
EVQLVESGGGLVQAGGSLRLSCAASGSTFSIDAMAWYRQAPGNTRELVAGISSGGRSHVADSVKGRFTISGDNAKNTVYLQMNNLKPEDTAVYYCKAAARVTEYSYEYDYWGQGTQVTVSS

SEQ ID NO: 573 - protein, 116 aa, Lama glama
QLQLVESGGGLVQAGGSLRLSCVLSGNTFGRNVITWYRQPPGKQREFIAMITSSGSTRYADSVKGRFTMSRDTAKNTAFLQMSSLKVEDTAVYYCWTAIAGQGEYWGQGTQVTVSS

SEQ ID NO: 574 - protein, 122 aa, Lama glama
QVQLVESGGGLVQAGGSLRLSCAASGRTFGSYVMGWFRQAPGKEREFVAAASRSTGNTVYPDSVKGRFTISRDDAKNTLYLQMNSLKPEDTAVYYCAARASINYNYANWPYWGQGTQVTVSS

SEQ ID NO: 575 - protein, 123 aa, Lama glama
QVQLVESGGGLVQAGGSLRLSCAASGTIESINDMGWYRQAPGKQRELVATVSSSGMTNYADSAKGRFTISRDNAKNTMYLQMNSLKPEDTAVYYCKAHISRSYYYDSLGYDYWGQGTQVTVSS

SEQ ID NO: 576 - protein, 121 aa, Lama glama
EVQLVESGGGLVQAGDSLRLSCARSGLTASTYAMAWFRQAPGKEREFVSVIDWKGDTTYANSVKGRFTISRDNGKNTVYLQMNSLQPEDTALYYCAARGSPAYVARLYAYWGRGTQVTVSS

SEQ ID NO: 577 - protein, 123 aa, Lama glama
QLQLVESGGGLAQAGGSLRLSCAASGRTFVGSYVMGWFRQAPGKEREFVAASSRSTGNTVYLDSVKGRFTISRDDAKNTLFLQMNRLKPEDTAVYYCATRASVNYNYAAWPYWGQGTQVTVSS

SEQ ID NO: 578 - protein, 122 aa, Lama glama
QLQLVESGGGLVQAGGSLRLSCAASGRTVGSYAMGWFRQAPGKEREFLAASSRSTGNTVYADSVKGRFTISRDDAKNTLYLQMNSLKPEDTAVYYCAARASINYNYANWPYWGQGTQVTVSS

SEQ ID NO: 579 - protein, 121 aa, Lama glama
QLQLVESGGGLVQAGDSLRLSCTSSGLTASTYAMAWFRQAPGKEREFVAVVDWKGDTTYANSVKGRFTLSRDNGKNTNYLQMNSLQPEDTALYYCAARGSPGYVARLYAHWGRGTQVTVSS

SEQ ID NO: 580 - protein, 122 aa, Lama glama
QVQLVESGGGLVQAGGSLRLSCAASGRTVGSYVMGWFRQAPGKERELLAAFSRSTGNTVYADSVKGRFTISRDDAKNTLYLQMNSLKPEDTAVYYCAARASINYNYANWPYWGQGTQVTVSS

SEQ ID NO: 581 - protein, 121 aa, Lama glama
QLQLVESGGGLVQAGGSLRLSCTASGSTVRVTAGGWYRQAPGNERELVADIDNGGRPTYADSVKGRFTISRDNAKNAVDLQMNNLKPEDTAVYYCFVIAIPDSAPRPRAYWGQGTQVTVSS

SEQ ID NO: 582 - protein, 122 aa, Lama glama
QLQLVESGGGLVQAGGSLRLSCAASGFTLDDYAIGWFRQAPGKEREGVSCIASSDGSTYYADSVKGRFTISTDNAKNTVYLQMNSLKPEDTAVYYCARGGLLLPKPELFDSWGQGTQVTVSS

SEQ ID NO: 583 - protein, 115 aa, Lama glama
QLQLVESGGGLVQAGGSLRLSCVASGTTFTIEDIGWHRQAPGKQLELVAVSRGGGNADYSDSVKGRFTISRDNAKTTVYLQMSSLKPEDTAVYYCIAKTKATTKWGRGIQVTVSS

SEQ ID NO: 584 - protein, 115 aa, Lama glama
QVQLVESGGGLVQPGRSLRLSCAASGFTFSTKWMYWVRQGPGKGLERVAAIDAGGGSTYYADSVKGRFTISRDNSENTVYLQMNSLESADTAVYYCARSEGGLFRGRGTQVTVSA

SEQ ID NO: 585 - protein, 115 aa, Lama glama
QLQLVESGGGLVQPGGSLRLSCAASGFTFSNKWMYWVRQGPGKGLEWVSTIDASGGRTWYADSVKGRFTISRDNTKNTLYLQMNSLKSADAAVYYCARSAGGESRGRGTQVTVSS

SEQ ID NO: 586 - protein, 122 aa, Lama glama
QVQLVESGGGLVQAGDSLRLSCAASTRTVGTYVMGWFRQAPGKERELVAASSRSTGNTVYPDSVKGRFTVSRDDAKNALYLQMNSLKPEDTAIYYCAARASINYNYANWPYWGQGTQVTVSS

SEQ ID NO: 587 - protein, 123 aa, Lama glama
QVQLVESGGGLVQAGGSLRLSCAASGRTFVGTYVMGWFRQAPGKAREFVAASSRSTGNTVYLDSVKGRFTVSRDDAKNTLYLQMNSLKPEDTAVYYCATRASINYNYANWPYWGQGTQVTVSS

SEQ ID NO: 588 - protein, 115 aa, Lama glama
QLQLVESGGDLVQPGGSLRLSCAASGFTFSTKWMYWVRQGPGKGLERVAAIDAGGTSTYYADSVKGRFTISRDNSENTVYLQMNSLESADTAVYYCARSEGGLFRGRGTQVTVSA

SEQ ID NO: 589 - protein, 122 aa, Lama glama
QLQLVESGGGLVQAGGSLRLSCAASGFTLDDYAIGWFRQAPGKEREGVSCIATSDGSTYYADSVKGRFTISSDAAKNTVYLQMDSLKPEDTAVYYCARGGLLLPKPELFDGWGQGTQVTVST

SEQ ID NO: 590 - protein, 118 aa, Lama glama
QLQLVESGGGLVQAGGSLRLSCTASGSTSSIVIISWYRQAPGEQRDLVATINNGGDVDVADFVKGRFTISRDNAKNAVYLQMNNLKPEDTAVYYCSARTPSLRVTWFWGQGTQVTVSS

SEQ ID NO: 591 - protein, 123 aa, Lama glama
QVQLVESGGGLVQAGGSLRLSCAASGRTFVGSYVMGWFRQAPGKEREFVAASSRSTGNTVYLDSVKGRFTVSRDDAKNTLYLQMNSLKPEDTAVYYCATRASINYNYAAWPYWGQGTQVTVSS

SEQ ID NO: 592 - protein, 121 aa, Lama glama
QLQLVESGGGLVQAGDSLRLSCVFSGLTASTYAMAWFRQAPGKEREFVSVIDWKGDTTYANSVKGRFTISRDNGNKTVYLQMNSLQPEDTALYYCAARGSPGYVARLYAHWGRGTQVTVSS

SEQ ID NO: 593 - protein, 120 aa, Lama glama
QVQLVESGGGLVQPGGSLRLSCAASTSTLDFAIMGWYRQAPGKLRELVAVINNGGDADYTEDVKGRFTISRDNAKNTIYLQMNSLKPEDTAVYYCNAKKVKPSLFTNTYWGQGTQVTVSS

SEQ ID NO: 594 - protein, 122 aa, Lama glama
QVQLVESGGGLVQAGGSLRLSCAASGRTVGSYAVGWFRQAPGKEREFLAASSRSTGNTVYADSVKGRFTISRDDAKNTLYLQMNSLKPEDTAVYYCATRASINYNYANWPYWGQGTQVTVSS

SEQ ID NO: 595 - protein, 122 aa, Lama glama
QLQLVESGGGLVQAGGSLRLSCAASGRTVGSYAVAWFRQAPGKERELLAASSRSTGNTVYADSVKGRFTIARDDAKNTLYLQMNSLKPEDTAVYYCAARASINYNYANWPYWGQGTQVTVSS

SEQ ID NO: 596 - protein, 114 aa, Lama glama
QLQLVEFGGGLVQAGGSLRLSCAASGSTFSFNYMGWYRQAPGNQRDLVARISSGGITNYADSVKGRFTISGDNAKNTVYLQMNSLKPEDTARYYCFYNGYLNKWGQGTQVTVSS

SEQ ID NO: 597 - protein, 114 aa, Lama glama
QLQLVESGGGLVQAGGSLRLSCEASRTTFSFNYMGWYRQAPGNQRDLVARISSGGITNYADSVKGRFTISGDNAKNTVYLQMNSLKPEDTARYYCFYNGHLNKWGQGTQVTVSS

SEQ ID NO: 598 - protein, 123 aa, Lama glama
QLQLVESGGGLVQAGGSLRLSCAASGRTFVGSYVMGWFRQAPGKEREFVAASSRSTGNTVYLDSVKGRFTISRDDAKNMLYLQMNSLKPEDTAVYYCATRASINYNYANWPYWGQGTQVTVSS

SEQ ID NO: 599 - protein, 121 aa, Lama glama
QLQLVESGGGLVQAGGSLRLSCAASGSTVRVTAGGWYRQTPGNERELVADIDNGGSTTYADSVKGRFTISRDNAKNAVDLQMNSLKPEDTAVYRCFVIAIPDSAPRPRSYWGQGTEVTVSS

SEQ ID NO: 600 - protein, 121 aa, Lama glama
QVQLVESGGGLVQAGGSLRLSCAASGLTSSTYVMAWFRQAPGKEREFVSVIDSRGDTTYANSVKGRFTTSRDFGKNTVYLQMNSLQPEDTALYYCAARASPAYVARLYAYWGRGTQVTVSS

SEQ ID NO: 601 - protein, 114 aa, Lama glama
QLQLVESGGGLVQAGGSLRLSCVASGTTFTIEDIGWYRLAPGKQRELVAIISGGNTNYADSVKGRFTISRDNAKNTVYLQMNSLKPEDTAVYYCNGRTKATVKWGRGTQVTVSS

SEQ ID NO: 602 - protein, 114 aa, Lama glama
QVQLVESGGGLVQAGGSLGLSCAASGTTFSFVYMGWYRQAPGNQRDLVARISSDGSTNYADSVKGRFTISGDNAKNTVYLQMNNLKPEDTARYYCFYNGHVNKWGQGTQVTVSS

SEQ ID NO: 603 - protein, 123 aa, Lama glama
EVQLVESGGGLVQAGGSLRLSCAASGSISSINDMGWHRQAPGKQRELVAAISSSGSTNYADSVKGRFTISRDNAENTVYLQMNDLKPEDTAVYYCNAHISRSYYYDSEGYRYWGQGTQVTVSL

SEQ ID NO: 604 - protein, 123 aa, Lama glama
QLQLVESGGGLVQAGGSLRLSCAASGRTFVGSYVMGWFRQAPGKEREFVAASSRSTGNTVYLDSVKGRFTISRDDAKNTLFLQMNRLKPEDTAVYYCATRASVNYNYAAWPYWGQGTQVTVSS

SEQ ID NO: 605 - protein, 114 aa, Lama glama
QLQLVESGGGLVQAGGSLRLSCAASGTTFSFVYMGWYRQAPGNQRELVARLSSDGSTNYADSVKGRFLISGDNAKNTVYLQMNSLKPEDTARYYCFYNGHLNKWGQGTQVTVSS

SEQ ID NO: 606 - protein, 115 aa, Lama glama
QLQLVESGGGLVQPGGSLRLSCAASGFTFSTKWMYWVRQGPGKGLERVAAIDASGGSTYYADSVKGRFTISRDNSKNTLYLQMNSLESADTAVYYCARSEGGQFRGRGTQVTVSS

SEQ ID NO: 607 - protein, 118 aa, Lama glama
QLQLVESGGGLVQAGGSLRLSCAASGTTFDMNSIGWYRQAPGKQRELVATISIGGSTNYADSVKGRFTISRGNAKNTVYMQMNSLKPEDTAVYYCNAERIWPNRRTFWGQGTQVTVSS

SEQ ID NO: 608 - protein, 121 aa, Lama glama
EVQLVESGGGLVQAGGSLRLSCTGSGSTFSVTAGGWYRQAPGKQRELVADIANDGSTTYLDFVKGRFTISRDNAKNSVDLQMNSLKPEDTAVYYCFVVGISDSTPRPRAYWGQGTQVTVSS

SEQ ID NO: 609 - protein, 122 aa, Lama glama
QVQLVESGGGLVQPGGSLRLSCAASGFSLDYYAIGWFRQAPGKEREGVSSIASSDGSTYYADSVKGRFTISSDEAKNTVYLHMNSLKPEDTAVYYCARGGSLVLRPGHYDYWGQGTQVTVSS

SEQ ID NO: 610 - protein, 121 aa, Lama glama
QLQLVESGGGVVQAGGSLRLSCAASGLTSSTYVMAWFRQAPGKEREFVSVIDSRGDTTIAKSVKGRFSISRENGKNTVYLQMNSLQPEDTALYYCAARGSASYVARLYPYWGRGTQVTVSS

SEQ ID NO: 611 - protein, 121 aa, Lama glama
QVQLVESGGGLVQAGGSLRLSCTASGLTSSTYVMAWFRQAPGKEREFVSVIDSSGGTTYANSAKGRFTISRANGENTVYLQMNSLQPEDTALYYCAARGAISYVARLYAYWGRGTQVTVSS

SEQ ID NO: 612 - protein, 122 aa, Lama glama
QVQLVESGGGLVQAGGSLRLSCAASGRTVGTYAIGWFRQAPGKEREFVAAFSRSTGNTVYPDSVKGRFTISRDDAKNTLYLQMNSLKPEDTAVYYCAARASINYNYANWPYWGQGTQVTVSS

SEQ ID NO: 613 - protein, 122 aa, Lama glama
QLQLVESGGGLVQAGGSLRLSCAASGFTLDDYAIGWFRQAPGKEREGVSCIASSDGSTYYADSVKGRFTISTDNAKNTVYLQMNSLKPEDTAVYYCARGGLLLPKPELFEYWGQGTQVTVSS

SEQ ID NO: 614 - protein, 122 aa, Lama glama
QLQLVESGGGLVQAGGSLRLSCTASGRTVGTYVIGWFRQAPGKEREFVAALGRNTGNTVYPDSVKGRFTISKDDAKNTLYLQMNSLKPEDTAVYYCAARASINYNAANWPYWGQGTQVTVSS

SEQ ID NO: 615 - protein, 122 aa, Lama glama; identical to SEQ ID NO: 523

SEQ ID NO: 616 - protein, 114 aa, Lama glama
QLQLVESGGGLVQAGGSLRLSCAASGTTFSFVYMGWYRQAPGNQRDLVARISSDGSTNYADSVKGRFTISGDNAKNTVYLQMNSLKPEDTARYYCFWNGHTNKWGQGTQVTVSS

SEQ ID NO: 617 - protein, 122 aa, Lama glama
QLQLVESGGGLVQAGGSLRLSCAASGRTFGTYVIGWFRQAPGKEREFVAAFSRSTGNTVYPDSVKGRFTISRDDAKNTVYLQMNSLKPEDTAVYYCAARASINYNYANWPYWGQGTQVTVSS

SEQ ID NO: 618 - protein, 115 aa, Lama glama
QLQLVESGGGLVEAGGSLRLSCAVSGTTFSANTMVWHRQAPGKQREWVARISSSGTTNYADSVRGRFSLSRGNAKNTVYLQMNSLKPDDTAVYYCNAILPSRSIWGRGTQVTVSS

SEQ ID NO: 619 - protein, 116 aa, Lama glama
EVQLVESGGGLVQAGESLRLSCVLSGSTFGRNVITWYRQPPGKQREFIAMITTSGNTRYADSVKGRFTMSRDTAKNTAFLQMSNLKLEDTAVYYCWTAIAGQGEYWGQGTQVTVSS

SEQ ID NO: 620 - protein, 118 aa, Lama glama
QLQLVESGGGLVQPGGSLRLSCAASGTTSSLNLMGWYRQAPGKKRESVAHMTSDGRTDYVDSVKGRFTISRDNSKNTVYLQMNNLKPEDTAVYICYANPFLGSWSNYWGQGTQVTVSS

SEQ ID NO: 621 - protein, 115 aa, Lama glama
QLQLVESGGGLVQPGGSLRLSCAASGFTFSTKWMYWVRQGPGKGLERVAAIDAGGTSTYYADSVKGRFTISRDNSENTVYLQMNSLESADTAVYYCARSEGGLFRGRGTQVTVSA

SEQ ID NO: 622 - protein, 118 aa, Lama glama
QLQLVESGGGLVQAGGSLRLSCAASGTTSSLNLMGWYRQAPGKKRESVAHMTSDGDTDYVDSVKGRFTISRDNSKNTVYLQMNNVKPEDTAVYYCYANPFLGSWSNYWGQGTQVTVSS

SEQ ID NO: 623 - protein, 111 aa, Lama glama
QLQLVESGGGEVQAGESLRLSCAASGSTFSINVFAWYRDYPGKQRELVAVIGRSGTPRYADSVKGRFTISRDNAKNTVYLQMNSLEPEDTAVYYCHLGDTWGQGTQVTVSS

SEQ ID NO: 624 - protein, 121 aa, Lama glama
QLQLVESGGGLVQAGGSLRLSCTASGSTVRVTAGGWYRQTPGNERELVADIDNGGSTTYADSVKGRFTISRDNAKNAVDLQMNSLKPEDTAVYRCFVIAIPDSAPRPRAYWGQGTEVTVSS

SEQ ID NO: 625 - protein, 122 aa, Lama glama
QLQLVESGGGLVQAGGSLRLSCAASGFTLDDYAIGWFRQAPGKEREGVSSIATSDGSTYYADSVKGRFTISSDNAKNTVYLQMNSLKPEDTAVYYCARGGLLLPRPELYEYWGQGTQVTVSS

SEQ ID NO: 626 - protein, 115 aa, Lama glama
QLQLVESGGGLVQAGGSLRLSCAVSASTSSINLMGWYRQAPGKRRDVVAVINSGGDTDYADSVKGRFTISRDNAKNTIYLQMNSLKSEDTAVYYCYTAIGWRNYWGQGTQVTVSS

SEQ ID NO: 627 - protein, 122 aa, Lama glama
QLQLVESGGGLVQAGGSLRLSCAASGRTFGSYVMGWFRQAPGKERAFVGAASRSTGTTVYLDSVKGRFTISRDDAKNTLYLQMNSLKPEDTAVYYCAARASINYNYANWPYWGQGTQVTVSS

SEQ ID NO: 628 - protein, 116 aa, Lama glama
EVQLVESGGGVVQAGGSLRLSCVLSGNTFGDNVITWYRQPPGKQREFIAMITKSGSTRYADSVKGRFTMSRDTAKNTAFLQMSSLKVEDTAVYYCWTAIAGQGEYWGQGTQVTVSS

SEQ ID NO: 629 - protein, 114 aa, Lama glama
QVQLVESGGGLVQAGGSLRLSCVAYGSTFSINVFAWYRQAPGEQRELGELVAFIGRSGTTKYGDSVKGRFTISRDNAKNTVYLQMDSLRPEDTAVYYCNLGDYWGQGTQVTVSS

SEQ ID NO: 630 - protein, 111 aa, Lama glama
QLQLVESGGGLVQAGGSLRLSCAASGSTFSINVFAWYRQGSGEQRELVAFIGRSGTARYGDSVKGRFSISRDNAKKTVYLQMDSLRPEDTAVYYCNLGDYWGQGTQVTVSS

SEQ ID NO: 631 - protein, 116 aa, Lama glama
QLQLVESGGGLVQAGESLRLSCVLSGSTFGRNVITWYRQPPGKQREFIAMITTSGSTRYADSVKGRFTMSRDTAKNTAFLQMSNLKLEDTAVYYCWTAIAGQGEYWGQGTQVTVSS

SEQ ID NO: 632 - protein, 111 aa, Lama glama
QLQLVESGGGEVQAGESLRLSCAASGSTFSINVFAWYRDYPGKQRELVAVIGRSGTPRYADSVKGRFTISRDNVRNTVALQMNSLEPEDTAVYYCHLGDTWGQGTQVTVSS

SEQ ID NO: 633 - protein, 461 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 634 - protein, 405 aa, Homo sapiens
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 635 - DNA, 24 bp, Lama glama
  source 1..24: mol_type = other DNA
ggaagcaccttccctatcaatgtc

SEQ ID NO: 636 - DNA, 24 bp, Lama glama
  source 1..24: mol_type = other DNA
ggacgccccatcagtgcctataca

SEQ ID NO: 637 - DNA, 24 bp, Lama glama
  source 1..24: mol_type = other DNA
ggaactatcgagagtatcaatgac

SEQ ID NO: 638 - DNA, 24 bp, Lama glama
  source 1..24: mol_type = other DNA
ggaagcacgttcaatttccgtatc

SEQ ID NO: 639 - DNA, 24 bp, Lama glama
  source 1..24: mol_type = other DNA
ggaagcatcttcacgaccgctacc

SEQ ID NO: 640 - DNA, 24 bp, Lama glama
  source 1..24: mol_type = other DNA
gggagcatcggaagactcaatgtc

SEQ ID NO: 641 - DNA, 24 bp, Lama glama
  source 1..24: mol_type = other DNA
ggaacgacgttcgatatgaattcc

SEQ ID NO: 642 - DNA, 24 bp, Lama glama
  source 1..24: mol_type = other DNA
ggacgcaccttcagtagcggcgcc

SEQ ID NO: 643 - DNA, 24 bp, Lama glama
  source 1..24: mol_type = other DNA
ggacgcaccttcgtcggtagctat

SEQ ID NO: 644 - DNA, 24 bp, Lama glama
  source 1..24: mol_type = other DNA
ggaaccatcgaaagtatcaatgac

SEQ ID NO: 645 - DNA, 27 bp, Lama glama
  source 1..27: mol_type = other DNA
ggacgcaccttcgtcggtagctatgtc

SEQ ID NO: 646 - DNA, 24 bp, Lama glama
  source 1..24: mol_type = other DNA
ggactcaccgccagtacctatgcc

SEQ ID NO: 647 - DNA, 24 bp, Lama glama
  source 1..24: mol_type = other DNA
ggaacgactttcacgatcgaagac

SEQ ID NO: 648 - DNA, 24 bp, Lama glama
  source 1..24: mol_type = other DNA
ggaagcaccagcagtatcgttatc

SEQ ID NO: 649 - DNA, 24 bp, Lama glama
  source 1..24: mol_type = other DNA
ggaaccaccttcagtttcgtttac

SEQ ID NO: 650 - DNA, 24 bp, Lama glama
  source 1..24: mol_type = other DNA
ggaaccaccttcagtgccaatacc

SEQ ID NO: 651 - DNA, 24 bp, Lama glama
  source 1..24: mol_type = other DNA
ggaaccacctcaagcctcaatctc

SEQ ID NO: 652 - DNA, 24 bp, Lama glama
  source 1..24: mol_type = other DNA
ggattcaccttcagtaccaaatgg

SEQ ID NO: 653 - DNA, 24 bp, Lama glama
  source 1..24: mol_type = other DNA
ggaagcaccttcagtatcaatgtc

SEQ ID NO: 654 - DNA, 24 bp, Lama glama
  source 1..24: mol_type = other DNA
ggaagcaccgtcagggtaactgcc

SEQ ID NO: 655 - DNA, 24 bp, Lama glama
  source 1..24: mol_type = other DNA
ggaagcaccttcggtcgcaatgtc

SEQ ID NO: 656 - DNA, 30 bp, Lama glama
  source 1..30: mol_type = other DNA
gtcgcatttattggtaggagtggaaccaca

SEQ ID NO: 657 - DNA, 24 bp, Lama glama
  source 1..24: mol_type = other DNA
attacctcgagtgctggtaccaca

SEQ ID NO: 658 - DNA, 21 bp, Lama glama
  source 1..21: mol_type = other DNA
attaccagtggtggtagtaca

SEQ ID NO: 659 - DNA, 21 bp, Lama glama
  source 1..21: mol_type = other DNA
attaatagtggtggtagcgcc

SEQ ID NO: 660 - DNA, 21 bp, Lama glama
  source 1..21: mol_type = other DNA
attactactggtggtagcaca

SEQ ID NO: 661 - DNA, 21 bp, Lama glama
  source 1..21: mol_type = other DNA
attagtagtgataataaaaaa

SEQ ID NO: 662 - DNA, 21 bp, Lama glama
  source 1..21: mol_type = other DNA
ataagtatgggaggcgacaca

SEQ ID NO: 663 - DNA, 24 bp, Lama glama
  source 1..24: mol_type = other DNA
attaggtggagtgatgagcgcaca

SEQ ID NO: 664 - DNA, 24 bp, Lama glama
  source 1..24: mol_type = other DNA
tcaagccggagtactggcaatacc

SEQ ID NO: 665 - DNA, 21 bp, Lama glama
  source 1..21: mol_type = other DNA
gttagtagtagtggtatgaca

SEQ ID NO: 666 - DNA, 24 bp, Lama glama
  source 1..24: mol_type = other DNA
tcaagccggagtactggtaatacc

SEQ ID NO: 667 - DNA, 21 bp, Lama glama
  source 1..21: mol_type = other DNA
gttgactggaaaggtgataca

SEQ ID NO: 668 - DNA, 21 bp, Lama glama
  source 1..21: mol_type = other DNA
tctcgtggtggtggtaacgca

SEQ ID NO: 669 - DNA, 21 bp, Lama glama
  source 1..21: mol_type = other DNA
attaataatggtggtgacgta

SEQ ID NO: 670 - DNA, 21 bp, Lama glama
  source 1..21: mol_type = other DNA
attagtagtgatggtagtaca

SEQ ID NO: 671 - DNA, 21 bp, Lama glama
  source 1..21: mol_type = other DNA
attagtagtagtggtacgaca

SEQ ID NO: 672 - DNA, 21 bp, Lama glama
  source 1..21: mol_type = other DNA
atgacgagtgatggtcggacg

SEQ ID NO: 673 - DNA, 24 bp, Lama glama
  source 1..24: mol_type = other DNA
attgatgctggtggtactagcaca

SEQ ID NO: 674 - DNA, 21 bp, Lama glama
  source 1..21: mol_type = other DNA
atcggtaggagtggtacgcca

SEQ ID NO: 675 - DNA, 21 bp, Lama glama
  source 1..21: mol_type = other DNA
attgataatggtggtagtaca

SEQ ID NO: 676 - DNA, 21 bp, Lama glama
  source 1..21: mol_type = other DNA
attacgactagtggtagcaca

SEQ ID NO: 677 - DNA, 16 bp, Lama glama
  source 1..16: mol_type = other DNA
aatctaggtgactact

SEQ ID NO: 678 - DNA, 36 bp, Lama glama
  source 1..36: mol_type = other DNA
gcagccgacgatcagggggataactggtatgaccac

SEQ ID NO: 679 - DNA, 51 bp, Lama glama
  source 1..51: mol_type = other DNA
aatgcagaggtcaagcgccattactattacgactctgctggctatcgctac

SEQ ID NO: 680 - DNA, 42 bp, Lama glama
  source 1..42: mol_type = other DNA
catgcaaaaaaagttgaaccaacattattcactacgacctac

SEQ ID NO: 681 - DNA, 33 bp, Lama glama
  source 1..33: mol_type = other DNA
aatgcagcgaccagattgtccagtactaactac

SEQ ID NO: 682 - DNA, 30 bp, Lama glama
  source 1..30: mol_type = other DNA
aatgtccgttggactatgggtggaaactac

SEQ ID NO: 683 - DNA, 36 bp, Lama glama
  source 1..36: mol_type = other DNA
aatgcagagcggatttggcctaatcgacgtaccttt

SEQ ID NO: 684 - DNA, 27 bp, Lama glama
  source 1..27: mol_type = other DNA
cggtcaaccgtcggcaacttactatac

SEQ ID NO: 685 - DNA, 45 bp, Lama glama
  source 1..45: mol_type = other DNA
gcaacaagggccagtattaattacaacgccgcgacttggccctac

SEQ ID NO: 686 - DNA, 51 bp, Lama glama
  source 1..51: mol_type = other DNA
aaggcccacattagtcgtagttactactacgatagcttagggtatgattac

SEQ ID NO: 687 - DNA, 42 bp, Lama glama
  source 1..42: mol_type = other DNA
acaagggccagtgttaattacaactacgcggcttggccctac

SEQ ID NO: 688 - DNA, 45 bp, Lama glama
  source 1..45: mol_type = other DNA
gcagccaggggaagccctggttacgtggccagactatatgcacac

SEQ ID NO: 689 - DNA, 27 bp, Lama glama
  source 1..27: mol_type = other DNA
attgctaagactaaagcgaccacaaag

SEQ ID NO: 690 - DNA, 36 bp, Lama glama
  source 1..36: mol_type = other DNA
agtgcacgcacaccttcactccgtgtgacctggttc

SEQ ID NO: 691 - DNA, 24 bp, Lama glama
  source 1..24: mol_type = other DNA
ttttggaatgggcacacgaacaag

SEQ ID NO: 692 - DNA, 27 bp, Lama glama
  source 1..27: mol_type = other DNA
aatgccattctaccgtctcgtagtata

SEQ ID NO: 693 - DNA, 36 bp, Lama glama
  source 1..36: mol_type = other DNA
tatgcaaacccgttcctcggtagctggtccaactac

SEQ ID NO: 694 - DNA, 24 bp, Lama glama
  source 1..24: mol_type = other DNA
gcaagatcagagggcgggctgttc

SEQ ID NO: 695 - DNA, 15 bp, Lama glama
  source 1..15: mol_type = other DNA
catctaggggacacc

SEQ ID NO: 696 - DNA, 45 bp, Lama glama
  source 1..45: mol_type = other DNA
tttgtaatagcgatccctgattctgcgcccagacctcgcgcctac

SEQ ID NO: 697 - DNA, 30 bp, Lama glama
  source 1..30: mol_type = other DNA
tggacggctatcgctggtcagggagagtac

SEQ ID NO: 698 - DNA, 342 bp, Lama glama
  source 1..342: mol_type = other DNA
cagttgcagctggtggagtctgggggaggcttggtgcaggctggggggtctctgagactctcctgtgcagcctctggaagcaccttccctatcaatgtcttcgcctggtaccgccaggctccaggggagcagcgcgagttaggcgagttggtcgcatttattggtaggagtggaaccacaaactatggagactccgtgaagggccgattcaccatctccagagacaacgccaagaacacagtgtatctgcaaatggacagcctgagacctgaagacacggccgtctattattgtaatctaggtgactactggggtcaggggacccaggtcaccgtctcctca

SEQ ID NO: 699 - DNA, 357 bp, Lama glama
  source 1..357: mol_type = other DNA
caggtgcagctggtggagtctgggggaggattggtgcaggctgggggctctctgagactctcctgtgcagcctctggacgccccatcagtgcctatacatgggcctggttccgccaggctccagggaaggagcgtgagtttgtagcagctattacctcgagtgctggtaccacatactatgcagactccgtaaggggccgattcaccatctccagagacaacgccgagaacacggtgtatctgcgaatgaacaccctgaaacctgaggacacggccgtttattactgcgcagccgacgatcagggggataactggtatgaccactggggccaggggacccaggtcaccgtctcctca

SEQ ID NO: 700 - DNA, 369 bp, Lama glama
  source 1..369: mol_type = other DNA
caggtgcagctggtggagtctgggggaggcttggtgcagcctggggggtctctgacactctcctgtgcagcctctggaactatcgagagtatcaatgacatgggctggtaccgccaggctccagggaagcagcgcgagttggtcgcagctattaccagtggtggtagtacaaactatgcagactccgtgaagggccgattcaccatctccagagacaacgccaagaacacggtgtatctgcaaatgaacagcctgaaacctgaggacacggccgtctattattgtaatgcagaggtcaagcgccattactattacgactctgctggctatcgctactggggccaggggacccaggtcaccgtctcctca

SEQ ID NO: 701 - DNA, 360 bp, Lama glama
  source 1..360: mol_type = other DNA
cagttgcagctcgtggagtctgggggaggcttggtgcaggctggggggtctctgagactctcctgtgcagcctctggaagcacgttcaatttccgtatcatgggctggtaccgccaggttccaggaaagcagcgcgagttggtcgcagttattaatagtggtggtagcgccgactatacagaggacgtgaagggccgattcaccatctccagagacgacgccaagagcacaatgtatctacaaatgaacagcctgaaacctgaagacacggccgtctattactgtcatgcaaaaaaagttgaaccaacattattcactacgacctactggggccaggggacccaggtcaccgtctcctca

SEQ ID NO: 702 - DNA, 351 bp, Lama glama
  source 1..351: mol_type = other DNA
caggtgcagctggtggagtctgggggaggcttggtgcagcatggggggtctctgagactgtcctgtgcagtctctggaagcatcttcacgaccgctaccatggcctggtaccgccaggctccggggaagcagcgcgactgggttgcagttattactactggtggtagcacacgctatgcagacgacgtgaagggccgattcgccatctccagagacaacgccaagaacacggtgtatctgcaaatgaacaacctgaaacctgaggacacggccgtctattactgtaatgcagcgaccagattgtccagtactaactactggggccaggggacccaggtcagcgtctcctca

SEQ ID NO: 703 - DNA, 348 bp, Lama glama
  source 1..348: mol_type = other DNA
cagttgcagctcgtggagtctgggggaggcttggtgcagcctggggggtctctgagactctcctgtgcagcctctgggagcatcggaagactcaatgtcatgggctggttccgccaggctccagggaagcagcgcgagttggtcgcagttattagtagtgataataaaaaagactatgcagactccgtgcagggccgattcaccatctccagagacaactccaagaacacggtgtatctgcaaatgaacagcctgaaacctgaggacacggccgtctactattgtaatgtccgttggactatgggtggaaactactggggccaggggacccaggtcaccgtctcctca

SEQ ID NO: 704 - DNA, 354 bp, Lama glama
  source 1..354: mol_type = other DNA
gaggtgcagctggtggagtctgggggaggcttggtgcaggcgggggggtctctgagactctcctgtgcggcctctggaacgacgttcgatatgaattccatgggctggtaccgccagggtccaggattcaagcgggagttggtcgcgactataagtatgggaggcgacacaaactatgcagactccgtgaagggccgattcacgatctccagagccaacgccaagaatacggtgtacatgcaaatgaacagcctgaaacctgaggacacggccgtctactactgtaatgcagagcggatttggcctaatcgacgtaccttttggggccaggggacccaggtcaccgtctcctca

SEQ ID NO: 705 - DNA, 348 bp, Lama glama
  source 1..348: mol_type = other DNA
cagttgcagctggtggagtctgggggaggattggtggaggctgggggctctctgagactctcctgtgcagcctctggacgcaccttcagtagcggcgccttgggctggttccgccaggctccagggaaggagcgtgagtttgtagcaactattaggtggagtgatgagcgcacacagtattcagagttcgtgaagggccgattcaccatctccagagacaacgccaagaacacggtgtatctgcagatgaacaacctccaacctgaggacacggccgtttattactgtcggtcaaccgtcggcaacttactatactggggccaggggacccaggtcaccgtctcctca

SEQ ID NO: 706 - DNA, 369 bp, Lama glama
  source 1..369: mol_type = other DNA
gaggtgcagctggtggagtctgggggaggattggtgcaggctgggggctctctgagactctcctgtgcagcctctggacgcaccttcgtcggtagctatgtcatgggctggttccgccaggctccaggaaaggagcgtgaatttgttgcagcttcaagccggagtactggcaataccgtctatttagactccgtgaagggtcgtttcaccgtctccagagacgacgccaagaacacgctgtatctgcaaatgaacagcctgaaaactgaggacacggccgtttattactgtgcaacaagggccagtattaattacaacgccgcgacttggccctactggggccaggggacccaggtcaccgtctcctcg

SEQ ID NO: 707 - DNA, 369 bp, Lama glama
  source 1..369: mol_type = other DNA
caggtgcagctggtggagtctgggggaggcttggtgcaggctggggggtctctgagactctcctgtgcagcctctggaaccatcgaaagtatcaatgacatgggctggtaccgccaggctccagggaagcagcgcgagttggtcgcaactgttagtagtagtggtatgacaaactatgcagactccgcgaaggggcgattcaccatctccagagacaacgccaagaacacaatgtatctacaaatgaacagcctgaaacctgaggacacggccgtctattactgtaaggcccacattagtcgtagttactactacgatagcttagggtatgattactggggccaggggacccaggtcaccgtctcctca

SEQ ID NO: 708 - DNA, 369 bp, Lama glama
  source 1..369: mol_type = other DNA
cagttgcagctcgtggagtctgggggaggattggcgcaggctgggggctctctgagactctcctgtgcagcctctggacgcaccttcgtcggtagctatgtcatgggctggttccgccaggctccaggaaaggagcgtgaatttgttgcagcttcaagccggagtactggtaataccgtctatttagactccgtgaagggtcgtttcaccatctccagagacgacgccaagaacacgctgttcctgcaaatgaaccgcctgaaacctgaggacacggccgtttattactgtgcaacaagggccagtgttaattacaactacgcggcttggccctactggggccaggggacccaggtcaccgtctcctca

SEQ ID NO: 709 - DNA, 363 bp, Lama glama
  source 1..363: mol_type = other DNA
cagttgcagctcgtggagtctgggggaggattggtgcaggctggggactcgttgagactctcctgcacatcctctggactcaccgccagtacctatgccatggcctggttccgccaggctccagggaaggagcgcgagtttgtggcagtagttgactggaaaggtgatacaacctatgcaaactccgtgaagggccggttcaccctctccagagacaacggcaagaacacgaactatctacaaatgaacagcctgcaacctgaggatacggccctttattactgtgcagccaggggaagccctggttacgtggccagactatatgcacactggggccgggggacccaggtcaccgtctcctca

SEQ ID NO: 710 - DNA, 345 bp, Lama glama
  source 1..345: mol_type = other DNA
cagttgcagctcgtggagtctgggggaggcttggtgcaggctggggggtctctgagactctcctgtgtagcctcaggaacgactttcacgatcgaagacatcggctggcaccgccaggctcccgggaagcagctcgaattggtcgcagtttctcgtggtggtggtaacgcagattattctgactccgtgaagggccgattcaccatctccagagacaacgccaagactacggtgtatctccaaatgagcagcctgaaacctgaggacacggccgtctattactgtattgctaagactaaagcgaccacaaagtggggccgggggatccaggtcactgtctcctca

SEQ ID NO: 711 - DNA, 354 bp, Lama glama
  source 1..354: mol_type = other DNA
cagttgcagctcgtggagtctgggggaggcttggtgcaggctggggggtctctgagactctcctgtacagcctctggaagcaccagcagtatcgttatcatcagttggtaccgccaggctccaggggagcagcgcgacttggtcgcaactattaataatggtggtgacgtagacgtggcagattttgtgaagggccgattcaccatctccagagacaacgccaagaacgcggtgtatctgcaaatgaacaacctgaaacctgaggacacggccgtctattattgtagtgcacgcacaccttcactccgtgtgacctggttctggggccaggggacccaggtcaccgtctcctca

SEQ ID NO: 712 - DNA, 342 bp, Lama glama
  source 1..342: mol_type = other DNA
cagttgcagctcgtggagtctgggggaggcttggtgcaggctggggggtctctgagactctcctgtgcagcctctggaaccaccttcagtttcgtttacatgggctggtaccgccaggctccagggaaccagcgcgacttggtcgcgcgtattagtagtgatggtagtacaaactatgcagactccgtgaagggccgattcaccatctccggagacaacgccaagaacacagtgtatctgcaaatgaacagcctgaaacctgaggacacggcccgctattactgtttttggaatgggcacacgaacaagtggggccaggggacccaggtcaccgtctcctca

SEQ ID NO: 713 - DNA, 345 bp, Lama glama
  source 1..345: mol_type = other DNA
cagttgcagctcgtggagtctgggggaggcttggtggaggctggggggtctctgagactctcctgtgcagtttctggaaccaccttcagtgccaataccatggtgtggcaccgccaggctcccgggaagcagcgcgagtgggtcgcacgcattagtagtagtggtacgacaaactatgcagactccgtgaggggccgattcagcctctccagaggcaacgccaagaacacagtgtatctgcaaatgaacagcctgaagcctgacgacacggccgtctattactgtaatgccattctaccgtctcgtagtatatggggacgggggacccaggtcaccgtctcctca

SEQ ID NO: 714 - DNA, 354 bp, Lama glama
  source 1..354: mol_type = other DNA
cagttgcagctcgtggagtctgggggaggcttggtgcagcctggggggtctctgagactctcctgtgctgcctctggaaccacctcaagcctcaatctcatgggctggtaccgccaggctccagggaagaagcgcgagtcggtcgcacatatgacgagtgatggtcggacggactatgtagactccgtgaagggccgattcaccatctccagagacaactccaagaataccgtgtatctgcaaatgaacaacctgaaacctgaggacacggccgtctatatctgctatgcaaacccgttcctcggtagctggtccaactactggggccaggggacccaggtcaccgtctcctca

SEQ ID NO: 715 - DNA, 345 bp, Lama glama
  source 1..345: mol_type = other DNA
cagttgcagctcgtggagtctgggggaggcttggtgcagcctggggggtctctgagactctcctgtgcagcctctggattcaccttcagtaccaaatggatgtattgggtccgtcagggaccggggaaggggctcgagcgggtcgctgctattgatgctggtggtactagcacatactatgcagactccgtgaagggccggttcaccatctccagagacaactccgagaacacggtgtatctacaaatgaacagtctggaatctgcggacacggccgtgtattactgtgcaagatcagagggcgggctgttccggggccgggggacccaggtcaccgtctccgca

SEQ ID NO: 716 - DNA, 333 bp, Lama glama
  source 1..333: mol_type = other DNA
cagttgcagctcgtggagtctgggggaggcgaggtgcaggctggggagtctctgagactctcctgtgcagcctctggaagcaccttcagtatcaatgtcttcgcctggtaccgcgactatccagggaagcagcgcgagttggtcgcagttatcggtaggagtggtacgccacgttatgcagactccgtgaagggccgattcaccatctccagagacaacgccaagaacacggtgtatctgcaaatgaacagcctggaacctgaggacacggccgtctattattgtcatctaggggacacctggggccaggggacccaggtcaccgtctcctca

SEQ ID NO: 717 - DNA, 363 bp, Lama glama
  source 1..363: mol_type = other DNA
cagttgcagctcgtggagtctgggggaggcttggtgcaggctggggggtctctgagactctcctgtacagcctctggaagcaccgtcagggtaactgccgggggctggtaccgccagactccagggaacgagcgcgaattggtcgctgatattgataatggtggtagtacaacttatgcagactccgtgaagggccgattcaccatctccagagacaacgccaagaacgcggtggatctgcaaatgaacagcctgaaacctgaggacacggccgtctatcgctgttttgtaatagcgatccctgattctgcgcccagacctcgcgcctactggggccaggggaccgaggtcaccgtctcctca

SEQ ID NO: 718 - DNA, 348 bp, Lama glama
  source 1..348: mol_type = other DNA
cagttgcagctcgtggagtctgggggaggcttggtgcaggctggggaatctctgagactctcctgtgtactctctggaagcaccttcggtcgcaatgtcattacctggtaccgccagcctccagggaagcagcgcgagttcatcgcgatgattacgactagtggtagcacacgttacgcagactccgtgaagggccgattcaccatgtccagagacaccgccaagaacacagcatttctgcaaatgagcaacctgaaactcgaagacacggccgtctattactgttggacggctatcgctggtcagggagagtactggggccaggggacccaggtcaccgtctcctca

(19) United States
(12) Patent Application Publication
(10) Pub. No.: US 2025/0163183 A1
(43) Pub. Date: May 22, 2025

(54) CAMELID ANTIBODIES AGAINST ACTIVATED PROTEIN C AND USES THEREOF

(71) Applicant: COAGULANT THERAPEUTICS CORPORATION, Dongdaemun-gu (KR)

(72) Inventors: Terry HERMISTON, Mill Valley, CA (US); Maxine BAUZON, Hercules, CA (US); Derek SIM, Burlingame, CA (US); Douglas M. SCHNEIDER, Layfayette, CA (US)

(21) Appl. No.: 18/843,107

(22) PCT Filed: Mar. 3, 2023
(86) PCT No.: PCT/US2023/063713

§ 371 Date: Aug. 30, 2024

Related U.S. Application Data
(60) Provisional application No. 63/316,927, filed on Mar. 4, 2022.

(51) Int. Cl. C07K 16/40; A61K 39/00

(52) U.S. Cl. CPC C07K 16/40; C12Y 304/21069; A61K 2039/505; C07K 2317/22; C07K 2317/53; C07K 2317/569; C07K 2317/76; C07K 2317/92; C07K 2317/94

(57) ABSTRACT

The present invention provides antibodies against activated protein C (APC). Certain disclosed antibodies inhibit the anticoagulant activity of APC while preserving its beneficial cytoprotective functions. The present invention also provides nucleic acids, vectors, and host cells for producing the antibodies disclosed herein, as well as methods of using the antibodies to treat medical conditions such as bleeding, sepsis, and inflammation.

### CROSS-REFERENCE TO RELATED APPLICATIONS

This application claims priority to U.S. Provisional Application No. 63/316,927 filed on Mar. 4, 2022, the contents of which are incorporated by reference in their entireties.

### SEQUENCE LISTING

This application includes a sequence listing in XML format titled “172085.00117_ST26. xml”, which is 643,566 bytes in size and was created on Feb. 21, 2023. The sequence listing is electronically submitted with this application via Patent Center and is incorporated herein by reference in its entirety.

### FIELD

This application concerns antibodies, antibody fragments, and other binders of activated protein C (APC) and related formulations, nucleic acids, methods of preparation, and methods of use.

### BACKGROUND

Protein C (PC) is a zymogen that is synthesized in the liver as a single-chain, 461-amino acid precursor (SEQ ID NO: 633) and secreted into the blood. Prior to secretion, the single-chain precursor is converted into a heterodimer by removal of a lysine-arginine dipeptide (amino acids 198-199 in SEQ ID NO: 633) and a 42-amino acid preproleader (amino acids 1-42 in SEQ ID NO: 633). The heterodimeric form (417 amino acids) consists of a light chain (155 amino acids, 21 kDa) and a heavy chain (262 amino acids, 41 kDa) linked by a disulfide bridge. The light chain contains one gamma-carboxy glutamic acid (Gla) domain (45 amino acids), two EGF-like domains (46 amino acids), and linker sequences, while the heavy chain contains a highly polar activation peptide (amino acids 200-211 in SEQ ID NO: 633) and a catalytic domain with a typical serine protease catalytic triad. Cleavage of the PC zymogen at the thrombin cleavage site leads to removal of the activation peptide and activation of PC to activated PC (APC; SEQ ID NO: 634; 405 amino acids).

Human PC undergoes extensive post-translational modifications, including glycosylation, vitamin K-dependent gamma-carboxylation, and gamma-hydroxylation. It contains four potential N-linked glycosylation sites: one in the light chain (Asn97) and three in the heavy chain (Asn248, Asn313, and Asn329). Its Gla domain contains nine Gla residues and is responsible for the calcium-dependent binding of PC to negatively charged phospholipid membranes. The Gla domain can also bind to endothelial protein C receptor (EPCR), which aligns thrombin and thrombomodulin on the endothelial membrane during PC activation.

PC activation occurs on the surface of endothelial cells in a two-step process. It requires binding of PC (via the Gla domain) to EPCR on endothelial cells, followed by proteolytic cleavage of PC by thrombin/thrombomodulin complexes at Arg12 of the heavy chain. This single cleavage liberates the 12-amino acid activation peptide, converting the zymogen PC into the active serine protease APC.

Under physiological conditions, APC circulates at very low concentrations (1-2 ng/ml or 40 pM) in human blood and has a half-life of 20-30 minutes, whereas PC circulates at a much higher concentration (3-5 μg/ml or 65 nM) and has a half-life of 6-8 hours.

The PC pathway plays a pivotal role in regulating coagulation and serves as a natural defense mechanism against thrombosis by amplifying an anticoagulant response as a coagulant response mounts. Upon injury, thrombin is generated to initiate coagulation. Thrombin also triggers an anticoagulant response by binding to thrombomodulin in the lining of the vascular surface, thereby promoting PC activation. As a result, APC generation is correlated with thrombin concentration and PC levels. APC functions as an anticoagulant by inactivating two coagulation cofactors (i.e., factor Va and factor VIIIa) via proteolytic cleavage, thereby inhibiting the generation of thrombin. APC also directly contributes to an enhanced fibrinolytic response via complex formation with plasminogen activator inhibitors.

Bleeding is a major problem, both in medical conditions and in connection with medical procedures. For example, bleeding causes complications following surgery, organ transplant, intracranial hemorrhage, aortic aneurysm, post-partum hemorrhage, trauma, and overdose of certain anticoagulants. In fact, the leading cause of death in humans 1-44 years of age is acute bleeding associated with traumatic injury. There are currently few options for treating life-threatening, non-compressible hemorrhage. Bleeding disorders may be treated by inhibiting APC to promote thrombin generation and coagulation. Thus, a first object of the present invention is to provide a safe and efficacious treatment for bleeding.

In addition to its anticoagulant functions, APC also induces cytoprotective effects via its anti-apoptotic, anti-inflammatory, and endothelial barrier stabilization activities, all of which contribute to the regenerative outcomes associated with APC (e.g., stimulation of neurogenesis, angiogenesis, and wound healing).

Ischemia is a life-threatening condition that results from an inadequate blood supply to an organ or tissue. An insufficient blood supply causes tissues to become starved of oxygen and can potentially result in a heart attack or stroke. In highly metabolically active tissues, such as the heart and brain, ischemia can cause irreversible damage in as little as 3-4 minutes. APC's cytoprotective effects include decreasing apoptosis and inhibiting expression of inflammatory mediators following ischemia. Thus, a second object of the present invention is to provide a safe and efficacious treatment for ischemia.

Studies of human diseases and animal models suggest that extracellular histones are involved in either the onset or development of inflammatory processes in various organs (Moiana et al., Clin Biochemistry 94 (2021) 12-19 at 15). Thus, histones are being studied as potential therapeutic targets in diseases in which inflammation and thrombosis play a key pathophysiological role. APC reduces the cytotoxicity of extracellular histones through histone proteolysis. This function of APC relies on a negatively charged exosite, which includes Glu330 and Glu333.

Extracellular histones are known to be major mediators of death in sepsis. Sepsis is a life-threatening condition that occurs when the body's response to infection causes injury to its own tissues and organs. Sepsis is a leading cause of morbidity and mortality in the United States, accounting for nearly 270,000 deaths annually. APC's cytoprotective effects include inhibiting histone toxicity associated with death in sepsis. Thus, a third object of the present invention is to provide a safe and efficacious treatment for sepsis.

Histones form part of neutrophil extracellular traps (NETs), networks of extracellular fibers, primarily composed of DNA from neutrophils. NETs have been shown to be involved in the pathogenesis and progression of cancer. Cancer is a leading cause of death worldwide, accounting for nearly 10 million deaths in 2020, or one in six deaths. NETs affect cancer via several mechanisms, including the establishment of an inflammatory microenvironment and interaction with other pro-tumor mechanisms such as inflammasomes and autophagy (Shao et al., Frontiers in Oncology vol. 11 article 714357 (2021) p. 1). Evidence suggests that NETs play a role in various cancer types, including breast, lung, colorectal, pancreatic, blood, neurological, and cutaneous cancers (Id. at p. 6). Thus, interventions that affect the PC pathway can potentially impact cancer, and a fourth object of the present invention is to provide a safe and efficacious treatment for cancer.

APC-based therapeutics have been under development for several years. Genetic engineering of APC has produced APC variants with a selective reduction of either its anticoagulant or cytoprotective activities. (Griffin et al., Blood 125: 2898, 2015). For example, one such variant, 3K3A-APC (Lys191-193Ala), has less than 10% of wild-type APC's anticoagulant activity while retaining its full cytoprotective activity, and has advanced to clinical trials for ischemic stroke. However, previous attempts to translate research observations into APC-based medicines have failed, including the initial attempts to use APC as a treatment for sepsis. While encouraging clinical trial results led to the approval of a recombinant APC protein-based drug (drotrecogin alfa (activated), recombinant human activated protein C (rhAPC), trade name Xigris™) in 2001 (N Engl J Med 344:699, 2001), the positive initial findings were not replicated in subsequent randomized clinical trials, and this drug has been withdrawn from the market.

One potential problem with using exogenous proteins such as APC and APC variants as therapeutics is the potential for the recipient's immune system to generate anti-drug antibodies (ADAs) against them. The production of ADAs can have dramatic consequences, such as loss of efficacy and neutralization of the drug or other adverse events (J Pharm Sci 110(7):2575-2584, 2021). Thus, a fifth object of the present invention is to provide an APC-based therapeutic that has a lower immunogenicity risk.

Antibodies to APC have been explored as a means to selectively enhance or inhibit different functions of APC (see, e.g., US Pat. App. Pub. 20150307625 to Zhao et al. published Oct. 29, 2015; and US Pat. App. Pub. 2018326053 to Egner et al. published Nov. 15, 2018). For example, Magisetty et al. reported that a murine antibody that selectively inhibits the anticoagulant activity of APC while preserving its cytoprotective activity markedly reduced the severity of joint bleeding in a mouse model of hemophilic arthropathy, whereas an antibody that inhibits both the anticoagulant and cytoprotective activities of APC did not (Blood (2022) blood.2021013119). In Zhao et al., two different antibodies to APC were assessed as an APC-based treatment for hemophilia (Nat Commun 11(1):2992, 2020). One of the antibodies, termed class I, targeted the active site of APC, while the other, termed class II, targeted an exosite of APC. When these antibodies were administered to monkeys, both increased thrombin generation and promoted plasma clotting. However, whereas the class II antibody was well tolerated, the class I antibody resulted in animal death. Yet, the class II antibody still partially inhibited the active site of APC, which suggests that it may partially inhibit the cytoprotective effects of APC. Thus, a sixth object of the present invention is to provide an anti-APC antibody that maintains or enhances the cytoprotective effects of APC.

### SUMMARY

In a first aspect, the present invention provides isolated antibodies that specifically bind to activated protein C (APC) and minimally bind to unactivated protein C (PC). In some embodiments, the antibodies comprise a CDR3 comprising an amino acid sequence selected from the group consisting of SEQ ID NOs: 317-474. In some embodiments, the antibodies comprise a heavy chain variable domain (V_{H}) selected from the group consisting of SEQ ID NOs: 475-632.

In a second aspect, the present invention provides nucleic acids encoding the antibodies disclosed herein.

In a third aspect, the present invention provides vectors comprising the nucleic acids disclosed herein.

In a fourth aspect, the present invention provides host cells comprising a nucleic acid or vector disclosed herein.

In a fifth aspect, the present invention provides methods of producing an antibody disclosed herein. The methods comprise: (a) culturing a host cell disclosed herein under conditions that result in production of the antibody, and (b) isolating the antibody from the host cell.

In a sixth aspect, the present invention provides pharmaceutical compositions comprising an antibody disclosed herein and a pharmaceutically acceptable carrier.

In a seventh aspect, the present invention provides methods for treating or preventing a condition in a subject. The methods comprise administering a therapeutically effective amount of an antibody disclosed herein, an antibody disclosed herein specifically bound to an exogenous APC protein or variant thereof, or a pharmaceutical composition disclosed herein to the subject. In some embodiments, the condition is a condition that can be treated or prevented by enchaining or inhibiting the anticoagulant function of APC. In other embodiments, the condition is a condition that can be treated or prevented by enhancing or inhibiting the cytoprotective functions of APC.

In an eighth aspect, the present invention provides uses of the antibodies disclosed herein as a medicament.

### BRIEF DESCRIPTION OF THE DRAWINGS

FIG. 1A-FIG. 1F show the results of an epitope binning and affinity analysis of nanobodies LP1-LP21 against human activated protein C (APC) by bio-layer interferometry (BLI). The binding of APC to nanobody was detected based on the spectral shift of the reflected light at the sensor surface loaded with nanobody. The biosensor tips with nanobody-APC complexes were then dipped into TPP-4885 Fab solution. FIG. 1A is a table of the results. Based on spectral shifts, 8 clones were found to not block TPP-4885 Fab binding to APC (top) and 13 clones were found to block binding. FIG. 1B shows the results of an additional binning experiment performed with the nanobodies that did not block TPP-4885 Fab binding to human APC. LP7, LP9, LP12, and LP18 were found to not compete with LP2, LP3, and LP10 for APC binding. Values presented in the table represent binding responses over background (red=competition between analyte and ligand for APC, orange=competition but with measurable analyte binding, green=no competition). FIG. 1C shows the results of a clustering analysis, which identified 2 clusters of nanobodies at an AU p-value cut-off of 95% (red=AU approximately unbiased p-values, green=bp bootstrap probability, grey=edge number). FIG. 1D includes a table summarizing the nanobody clusters (top) and a graph of the median CDR3 length of each cluster (bottom). FIG. 1E is a graph showing K_{D} measurements for LP1-LP21 generated using BLI. Each nanobody was tested against 5 different concentrations of APC. R² for these measurements ranged from 0.92 to 0.99. FIG. 1F is a representative sensogram generated for antibody LP6 in the presence of BSA.

FIG. 2A-FIG. 2C show the effect of nanobodies LP1-LP21 on APC anticoagulant activity. FIG. 2A is a standard curve of Protac®-APTT clotting time in protein C (PC)-depleted plasma diluted in normal human plasma (∘) to achieve PC levels ranging from 0.78% to 100%. The APTT clotting time of normal human plasma (•) and PC-depleted plasma (V) in the absence of Protac® are also shown. FIG. 2B is a graph comparing the Protac®-APTT clotting time in the presence of the nanobodies (300 nM) in two independent experiments (Screen 1 and Screen 2). A consistent rank order was observed in the shortening of the Protac®-APTT clotting. FIG. 2C is a graph showing the average Protac®-APTT clotting time and percent APC activity in plasma of each nanobody (duplicates in 2 independent experiments). This graph demonstrates that the nanobodies have a wide variety of effects on Protac®-APTT clotting time. Among the nanobodies, LP11, LP8, and LP20 exhibited the highest potency for inhibiting anticoagulant activity.

FIG. 3A-FIG. 3F show the effect of nanobodies LP1-LP21 on APC-mediated histone H3 cleavage. APC (50 nM) and nanobody (500 nM) were preincubated together for 30 minutes. After the incubation, the APC-nanobody mixtures were added to histone H3 (100 μg/mL). Over a period of 2 hours, samples were taken at different time points and added to reducing sample buffer for SDS-PAGE analyses. The 20 kDa molecular marker was used as a reference for normalization between gels. FIG. 3A is an SDS-PAGE gel showing a time course of histone H3 cleavage by APC in the absence nanobody (representative of 3 experiments). FIG. 3B is a graph of histone H3 cleavage versus time for 9 nanobodies that had a minimal effect on APC-mediated H3 cleavage. The percent of H3 cleavage was calculated via normalization with the H3 band intensity at time=0 min: 100%−(intensity_{t=x min}/intensity_{t=0 min}). FIG. 3C is an SDS-PAGE gel showing that the nanobody LP14 enhances H3 cleavage. FIG. 3D is a graph of histone H3 cleavage versus time for 12 nanobodies that inhibited APC-mediated H3 cleavage. FIG. 3E is an SDS-PAGE gel showing that the nanobody LP21 potently inhibits H3 cleavage. FIG. 3F is a table presenting the data shown in (B) and (D) as numerical values.

FIG. 4A-FIG. 4E show the effect of nanobodies LP1-LP21 on APC-mediated cleavage of SEAP-protease-activated receptor 1 (PAR1). FIG. 4A is a series of graphs showing normalized SEAP-PAR1 cleavage for each nanobody over a range of nanobody concentrations. APC and nanobodies were added to HEK293 cells expressing SEAP-PAR1 and EPCR. Nanobodies were found to modulate APC-mediated PAR1 cleavage to different degrees. LP8, LP17, LP19, and LP21 showed the most potent inhibition, while LP3, LP9, LP11, and LP18 showed minimal inhibition even at the highest concentration of 500 nM (n=3 independent experiments). PAR1 cleavage is expressed as the percentage of the total SEAP activity present on the cells versus background. FIG. 4B is a table describing the R41Q-SEAP-PAR1 and R46Q-SEAP-PAR1 cell lines that were used to evaluate the cleavage pattern of APC (50 nM) at R46 and R41, respectively. FIG. 4C is a series of graphs showing the R46/R41 cleavage ratio for the PAR1 cleavage-inhibiting nanobodies LP8 and LP20. These nanobodies inhibited APC cleavage at both R46 and R41 in a dose-dependent manner. At high concentrations of LP8 and LP20, the cleavage ratio shifted to below 1, i.e., a less cytoprotective PAR1 cleavage profile (n=3 independent experiments). FIG. 4D is a series of graphs showing the R46/R41 cleavage ratio for the PAR1 cleavage-sparing nanobodies LP11 and LP18, which induced a curvilinear cleavage response at both R46 and R41. The R46/R41 cleavage ratios were above 1 at all tested concentrations, suggesting that LP11 and LP18 may enhance cytoprotection by APC (n=3 independent experiments). FIG. 4E is a table presenting the data shown in (C) and (D) as numerical values.

FIG. 5A-FIG. 5H show the effect of LP11 on APC-mediated endothelial barrier function. FIG. 5A is a graph of normalized cell index (NCI) versus time of thrombin addition (n=4), which demonstrates the barrier protective effect of APC (40 nM) in the presence of various concentrations of LP11 against barrier permeability induced by thrombin (0.25 nM). FIG. 5B is a graph quantifying the data shown in (A) based on the lowest NCI value of each time course, with the APC effect in the absence of LP11 set to 100% and the thrombin alone group set to 0%. FIG. 5C is a graph showing transient endothelial barrier disruption shortly after APC-priming of the EA.hy926 cells (n=4). FIG. 5D is a graph quantifying the data shown in (C). FIG. 5E is a graph showing inhibition of the transient barrier disruption by APC-priming (40 nM) using PAR1 antagonists, vorapaxar (1 μM) or SCH79797 (1 μM) (n=3). FIG. 5F is a graph showing inhibition of the transient endothelial cell barrier disruption upon APC-priming (40 nM) in the presence of LP11 (n=4). FIG. 5G is a graph quantifying the data shown in (F). FIG. 5H is a graph showing the overall profile of the changes in NCI plotted against the time of APC addition to the cells (APC-priming).

FIG. 6A-FIG. 6B show the purified nanobodies (LP1-LP21) run on a reducing SDS-PAGE gel (12% Bis-Tris with MOPS running buffer). FIG. 6A shows a gel with LP1 to LP7. FIG. 6B shows a gel with LP8 to LP21.

FIG. 7 shows a schematic outlining the process by which nanobodies LP1-LP21 were selected from the nanobody library for further study.

FIG. 8 shows ELISA screening (n=2) of 158 unique APC-PPACK specific clones against APC, FXa-PPACK, and PBS-coated wells. A selective binding towards APC over FXa-PPACK and PC was observed for these clones. Twenty-one nanobodies, named LP1 to LP21, belonging to 18 sequence-diverse families were selected for further characterizations.

FIG. 9 shows the results of ELISA screening of nanobodies LP1-LP21 against APC, FVIIa, FIXa, and FXIa-coated wells. Selective binding for APC was observed for all 21 of these nanobodies (n=3 independent experiments). ELISA plates were coated with 100 μL 10 μg/mL APC, FVIIa, FIXa, or FXIa in PBS for 1 hour followed by blocking with 200 μL PBS 1% casein pH 7.2 for 1 hour. Plates were washed with PBS-T (0.05%, pH 7.2) 4 times. Nanobodies (100 μL at 2 μg/mL in PBS) were added to the plates. After 1 hour of incubation, plates were washed 4 times with PBS-T and incubated with 1:25,000 HRP-anti-V5 (100 μL). After 1 hour of incubation, plates were washed 4 times with PBS-T and the amount of nanobody binding to the APC-coated plates was detected using TMB substrate. The reaction was terminated using 100 μL 2 N H₂SO₄.

FIG. 10 shows the effect of nanobodies on S-2366 cleavage by APC. The effect of the nanobodies on the amidolytic activity of APC against the small chromogenic substrate S-2366 was measured by the change in absorbance at 405 nm over time in a reaction consisting of APC (20 nM), nanobody (0-80 nM), and S-2366 (1 mM) in HBS/0.1% BSA. APC-mediated S-2366 cleavage was enhanced by 8 nanobodies, partially inhibited by 5 nanobodies, and unaffected by 8 nanobodies (n=3).

FIG. 11 shows Lineweaver-Burke plots of APC-mediated S-2366 cleavage in the presence of nanobodies that enhanced or inhibited cleavage (n=3). Reactions consisted of APC (16.7 nM), nanobody (67 nM), and S-2366 (0.075-1.2 mM) in HBS/0.1% BSA.

FIG. 12 demonstrates that LP11 shortened the prolonged clotting time in plasma with FVIII inhibitor in the presence of Protac®, a PC activator (n=3).

FIG. 13A-FIG. 13C show a characterization of LP11 expressed in mammalian cells. FIG. 13A is an SDS-PAGE gel showing purified mammalian LP11. FIG. 13B is a graph of Protac®-APTT clotting time versus LP11 concentration, which shows inhibition of Protac®-APTT by mammalian LP11. FIG. 13C is a set of graphs showing the effect of mammalian LP11 on cleavage in R41Q-SEAP-PAR1 and R46Q-SEAP-PAR1 cell lines.

FIG. 14A-FIG. 14B summarize the library panning experiment. FIG. 14A is a table summarizing the library panning process. Successive rounds (R1, R2, R3, and R4) of selection were performed on plates coated at 4° C. overnight with decreasing APC-PPACK concentrations (100, 50, 10, and 1 nM) and an increasing number and duration of washes. Negative selections were performed with factor Xa-PPACK, PC, and empty wells blocked with 2% bovine serum albumin (BSA) to remove non-specific binders. Enrichment for APC-PPACK binders with minimal binding to factor Xa-PPACK and PC was confirmed via phage pool ELISA between rounds. Phage bound to APC-PPACK was eluted using 100 mM triethylamine, followed by neutralization with 1 M Tris-HCl pH 7.5. Eluted phage was used to infect TG1 cells. Outputs were titered and plated on 2YTCG (2YT medium; 50 μg/mL Carbenicillin, 2% glucose) plates and allowed to grow at 30° C. overnight. A second arm of panning (R2′ and R3′) was performed with more stringent negative selection for PC. CFU, colony forming unit. FIG. 14B is a table summarizing the phage pool output from panning rounds R2, R3, R4, and R3′.

FIG. 15 is a schematic comparing the structure of conventional antibodies to that of camelid antibodies and V_{H}H antibodies.

### DETAILED DESCRIPTION

The present disclosure relates to antibodies that specifically bind to activated protein C (APC). In preferred embodiments, the antibodies bind to the activated form of this enzyme (i.e., APC) and minimally bind to the zymogen form of this enzyme (i.e., protein C (PC)). In some embodiments, the disclosed antibodies inhibit the anticoagulant activity of APC while at least partially preserving or even enhancing the pleiotropic cytoprotective functions of APC. In some embodiments, the disclosed antibodies enhance the cytoprotective functions of APC while partially or completely preserving the anticoagulant activity of APC. The present invention also provides polynucleotides encoding these antibodies, pharmaceutical compositions comprising these antibodies, methods of making these antibodies, and methods for treating conditions by administering therapeutically effective amounts of these antibodies.

The anti-APC antibodies of the present invention offer several advantages over the recombinant APC protein therapeutics that are currently on the market or in development. For example, anti-APC antibodies have a lower risk of immunogenicity than therapeutics comprising exogenous APC proteins and variants thereof (e.g., 3K3A-APC). Anti-drug antibodies (ADAs) against exogenous APC proteins may cross-react with the endogenous PC/APC protein, resulting in an autoimmune response. For example, the APC variant 3K3A-APC from ZZ Biotech, LLC is in clinical trials where it is being administered to patients in a high number of repeated doses to treat ischemic stroke (ClinicalTrials.gov Trial Id. NCT02222714). This may effectively immunize patients, causing them to develop anti-APC antibodies that could cross-react with endogenous APC. In contrast, ADAs against anti-APC antibodies should not cross-react with endogenous PC/APC protein, making the use of anti-APC antibodies a safer therapeutic strategy. Further, depending on the selected antibody format, fewer doses of an anti-APC antibody may be required to achieve a desired therapeutic effect as compared to the number of doses required for an APC protein-based drug.

### Antibodies:

In a first aspect, the present invention provides antibodies that specifically bind to APC. As used herein, the term “antibody” refers to a protein that comprises at least one antigen-binding domain from an immunoglobulin. This term encompasses both full-length immunoglobulins and antigen-binding fragments thereof.

As stated above, the term “antibody” includes fragments of full-length immunoglobulins that comprise an antigen-binding domain. Examples of antigen-binding fragments include, without limitation, (i) Fab fragments, i.e., monovalent fragments consisting a heavy chain variable region (V_{H}), a light chain variable region (V_{L}), a constant domain of the κ light chain (CL), and a first constant domain of the heavy chain (CH1); (ii) F(ab′)2 fragments, i.e., bivalent fragments comprising two Fab fragments linked by a disulfide bridge at the hinge region; (iii) Fd fragments consisting of the V_{H} and CH1; (iv) Fv fragments consisting of the V_{L} and V_{H} of a single arm of an antibody; (v) dAb fragments (Nature 341:544-546, 1989), which consists of a V_{H}; (vi) isolated complementarity determining regions (CDRs); (vii) minibodies, diabodies, triabodies, tetrabodies, and kappa bodies (see, e.g., Protein Eng 10:949-57, 1997); (viii) fragments of camelid antibodies, including V_{H}H antibodies, V_{H}H dimers, and V_{H}H-Fc fusions; and (ix) fragments of cartilaginous fish antibodies, including VNAR antibodies.

In some embodiments, the antibody is selected from the group consisting of an IgG1 antibody, an IgG2 antibody, an IgG3 antibody, an IgG4 antibody, an IgM antibody, an IgA1 antibody, an IgA2 antibody, a secretory IgA antibody, an IgD antibody, an IgE antibody, and antigen-binding fragments thereof. In other embodiments, the antibody comprises an alternative scaffold, such as a scaffold that comprises non-immunoglobulin binding proteins (e.g., an Affibody®, Affilin™, Affimer®, Alphabody, Anticalin®, Atrimer, Avimer, Centyrin, DARPin®, Fynomer, Kunitz domain, OBody, Pronectin®, or repebody). In some embodiments, the antibodies are in the IgG4 format. This format offers a therapeutic advantage, as IgG4 is the only subclass of IgG that does not mediate common IgG effector functions, such as antibody-dependent cell-mediated cytotoxicity or complement dependent-cytotoxicity.

In preferred embodiments, the antibody is a nanobody. As used herein, the term “nanobody” refers to an antibody fragment consisting of a single monomeric variable antibody domain. A nanobody may also be referred to as a “single-domain antibody.” In conventional antibodies, the antigen-binding region consists of a heavy chain variable region (V_{H}) and a light chain variable region (V_{L}). Thus, nanobodies may be obtained from a single variable region (V_{H} or V_{L}) of a conventional antibody. However, nanobodies may also be obtained from a heavy-chain antibody. A “heavy-chain antibody” or “heavy-chain-only antibody” is an antibody that consists of two heavy chains and lacks the two light chains found in conventional antibodies. Heavy-chain antibodies bind antigens using only a V_{H} domain. Thus, the V_{H} domains of heavy-chain antibodies are great candidates for use as nanobodies. Heavy-chain antibodies are produced naturally by camelids (e.g., llamas, alpacas, and camels) and cartilaginous fish (e.g., sharks). Camelids produce heavy-chain antibodies from which single-domain antibodies called V_{H}H antibodies can be obtained, and cartilaginous fish produce heavy-chain antibodies, referred to as immunoglobulin new antigen receptor (IgNAR), from which single-domain antibodies called VNAR antibodies can be obtained. Thus, in some embodiments, the nanobody is a V_{H}H antibody or a VNAR antibody.

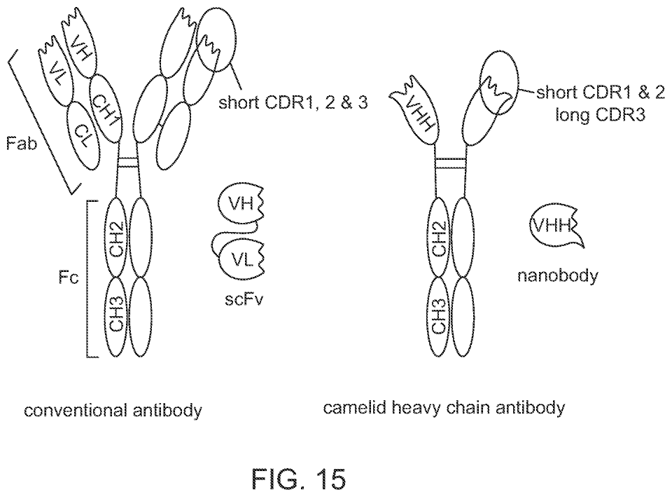

The antibody library that was screened by the inventors in the Examples was a V_{H}H antibody library produced in llamas. Thus, in preferred embodiments, the antibody of the present invention is a camelid V_{H}H antibody. Camelid antibodies comprise two heavy chains that lack the first constant Ig domain (CH1) found in typical heavy chains (FIG. 15). The variable portion of these camelid heavy chains is referred to as a single variable domain on a heavy chain (V_{H}H). Thus, nanobodies that consist of the V_{H}H portion of a camelid antibody are referred to as “V_{H}H antibodies.” V_{H}H antibodies have a similar structural architecture to V_{H} of typical human immunoglobulins and comprise four conserved framework regions (FR1/2/3/4) surrounding three hypervariable antigen-binding loops called complementarity determining regions (CDR1/2/3) (FIG. 15). Thus, the paratope of a V_{H}H antibody is comprised of (1) three CDRs that are in direct contact with the antigen and are involved in antigen binding and (2) four framework regions that maintain the overall structure. V_{H}H antibodies can be prepared using methods known in the art (see, e.g., U.S. Pat. Nos. 6,765,087, 6,838,254, WO 06/079372).

Therapeutics based on V_{H}H antibodies have recently been developed. For example, the first V_{H}H antibody-based drug, caplacizumab, was approved in 2018. V_{H}H antibodies are well-suited for use as therapeutics for several reasons. First, V_{H}H antibodies are extremely robust, offering a prolonged shelf life at both 4° C. and −20° C., resistance to proteolytic degradation and tolerance to increased temperature (60-80° C., or several weeks at 37° C.), exposure to non-physiological pH (pH range 3.0-9.0), elevated pressure (500-750 MPa), and chemical denaturants (2-3 M guanidinium chloride, 6-8 M urea). Notably, the robustness of V_{H}H antibodies has been attributed to their efficient refolding after chemical or thermal denaturation. Second, the monomeric structure of V_{H}H antibodies and their lack of post-translational modifications allow for their expression in microbial systems, including Escherichia coli, Saccharomyces cerevisiae, and Pichia pastoris. Consequently, V_{H}H antibodies can be manufactured at a low cost by producing them in milligram quantities per liter of culture in shaker flasks. Third, V_{H}H antibodies generated from camelids have a low immunogenic profile and are, thus, suitable for human administration. The sequence identity between V_{H}H antibodies and the V_{H} of human immunoglobulins of family III is above 80%. The camelid germline IGHV family 3 was found to have 95% sequence identity with its human FR counterpart. Nonetheless, the V_{H}H antibodies of the present invention can be “humanized” if desired. Fourth, V_{H}H antibodies offer increased flexibility for antigen-recognition as compared to traditional antibodies. Within a variable domain, CDR3 is the main contributor for antigen recognition and specificity, whereas CDR1 and CDR2 contribute to binding strength. The camelid V_{H}H antibody CDR3 is, on average, 18 amino acids long, which is substantially longer than the average 12- or 14-amino acid CDR3 of V_{H} domains from mouse or human antibodies, respectively. The extended CDR3 loops of V_{H}H antibodies can form finger-like structures or convex paratopes that can penetrate into small cavities or interact with concave surfaces on the surface antigens, respectively. This allows V_{H}H antibodies to target epitopes that are inaccessible to conventional antibodies. This increased flexibility allows for increased specificity, such that some V_{H}H antibodies can distinguish between different isoforms of the same protein. This increased specificity is useful for the development of antibodies that specifically recognize APC as opposed to PC, as these proteins differ by only 12 amino acids. Fifth, the small size of V_{H}H antibodies allows for high tissue penetration, and some nanobodies have even been reported to cross the blood-brain barrier.

The antibodies of the present invention are anti-APC antibodies. As used herein, the term “anti-APC antibody” refers to an antibody that specifically binds to an epitope of APC. Preferably, the antibodies specifically bind to human APC, which has the amino acid sequence of SEQ ID NO: 634, or a variant thereof. As used herein, the term “specific binding” refers to an ability to bind to a particular antigen (e.g., APC) in preference to other molecules. Typically, an antibody that exhibits specific binding binds to an antigen with an equilibrium dissociation constant (K_{D}) of at least about 10⁻⁵ M and binds to that antigen with an affinity that is higher (e.g., at least two-fold higher) than its binding affinity for an irrelevant protein (e.g., BSA, casein). Often, a higher affinity (i.e., lower K_{D}, e.g., in the low nanomolar range) translates to a more potent and specific therapeutic, as it increases the likelihood that the antibody will locate and bind to its target antigen.

The antibodies disclosed herein have a high specificity for the activated form of protein C (APC), as opposed to the inactive zymogen form (PC). This is useful given that there is a 1700-fold difference in plasma concentrations of APC (˜40 μM) and PC (70 nM). Thus, it is preferable that the antibodies of the present invention minimally bind to PC. An antibody that “minimally binds” to a particular antigen either (a) does not bind to the antigen at detectable levels, or (b) binds to the antigen with an equilibrium dissociation constant (K_{D}) that is lower than about 10⁻² M.

The term “protein C” or “PC” may refer to any variant, isoform, or homolog of the zymogen PC. Preferably, PC is human PC, which has the amino acid sequence of SEQ ID NO: 633, or a variant thereof.

The term “epitope” refers to the region of an antigen to which an antibody specifically binds. For examples of APC epitopes, see U.S. Patent Application Publication 2018/326053, which describes epitopes outside of the catalytic triad of the active site of human APC. Conversely, the term “paratope” refers to the area of the antibody to which the antigen specifically binds.

Antibody binding activities may be assessed using methods that are known in the art, including enzyme-linked immunosorbent assay (ELISA), surface plasmon resonance (SPR), radioimmunoassay, bio-layer interferometry (BLI), and the like. For example, to ensure that the antibodies disclosed herein have a suitable association constant (i.e., kₒₙ) for use as human therapeutics, they were assessed via BLI analysis.

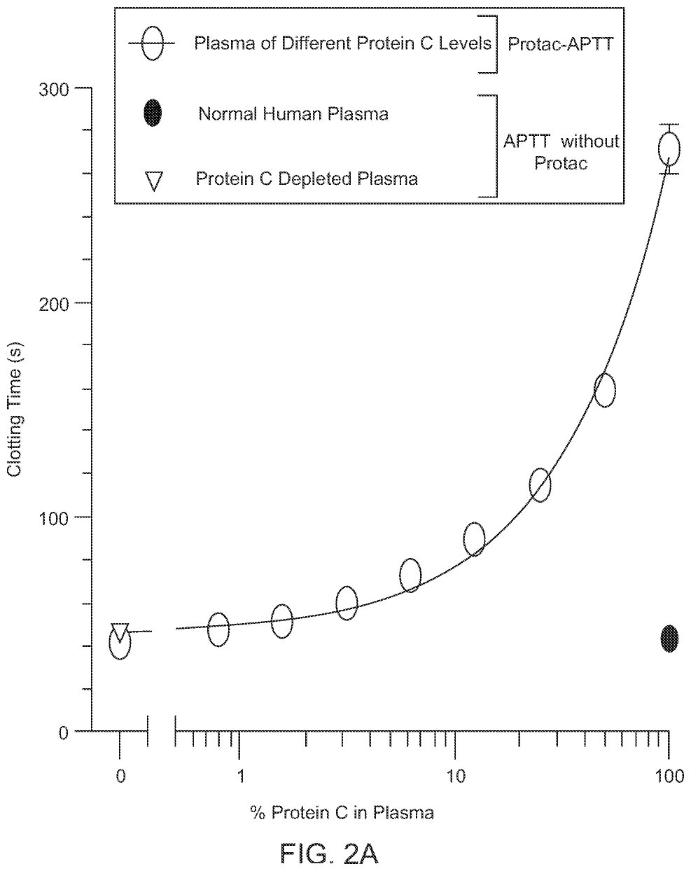

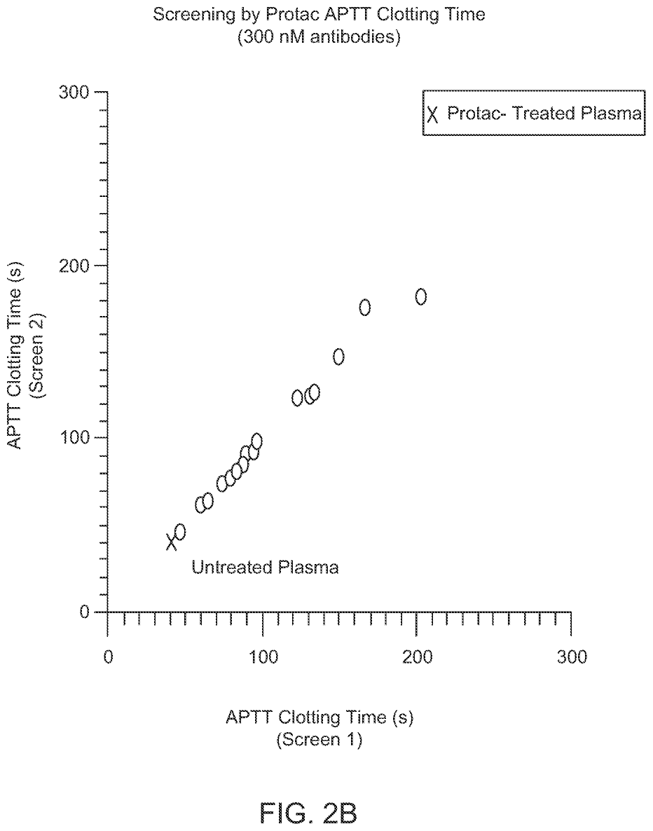

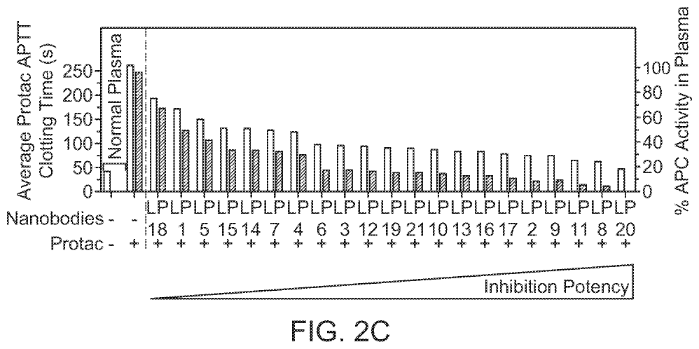

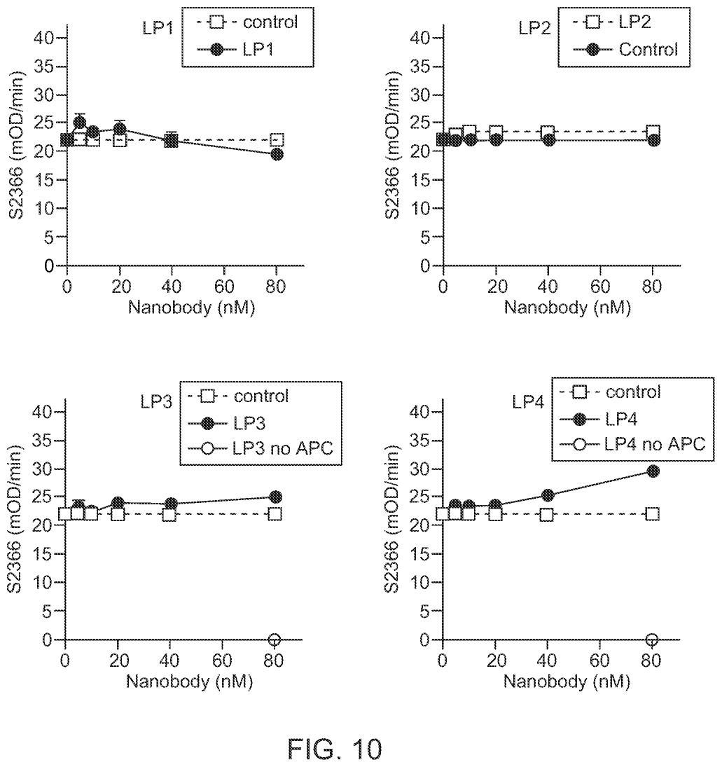

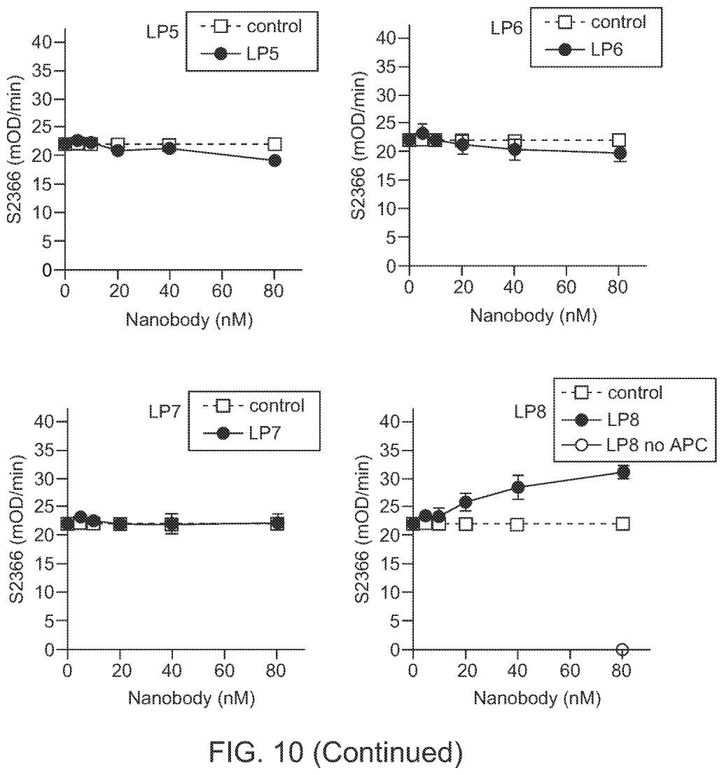

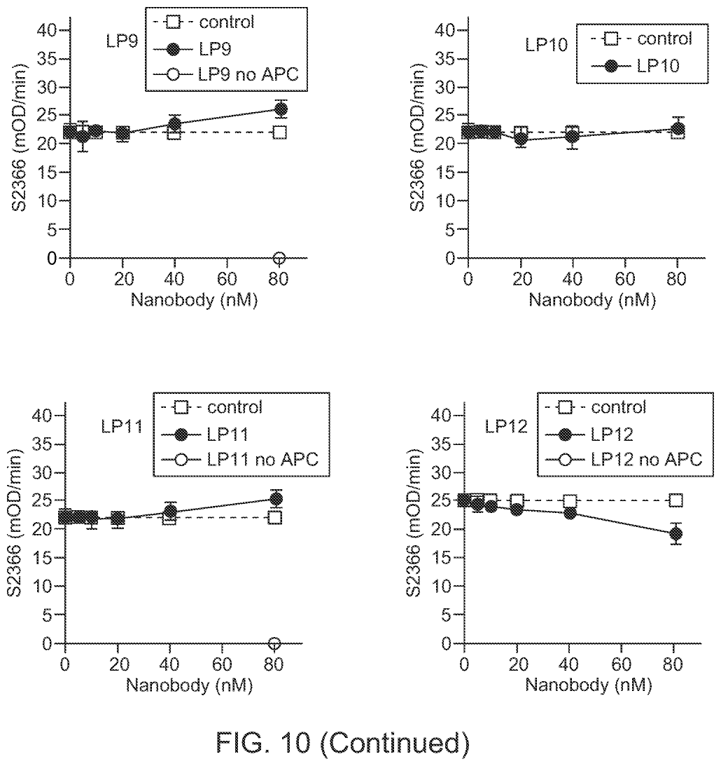

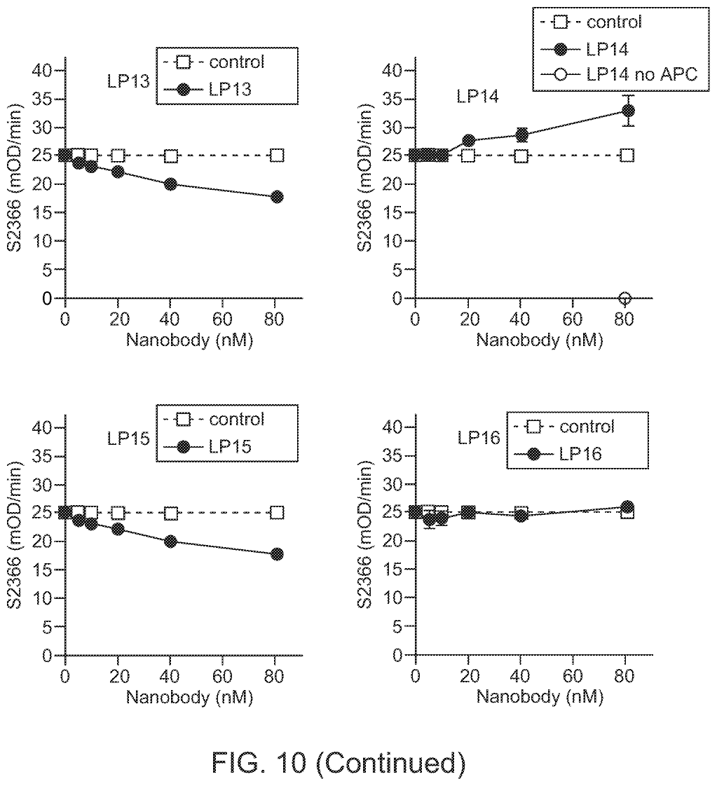

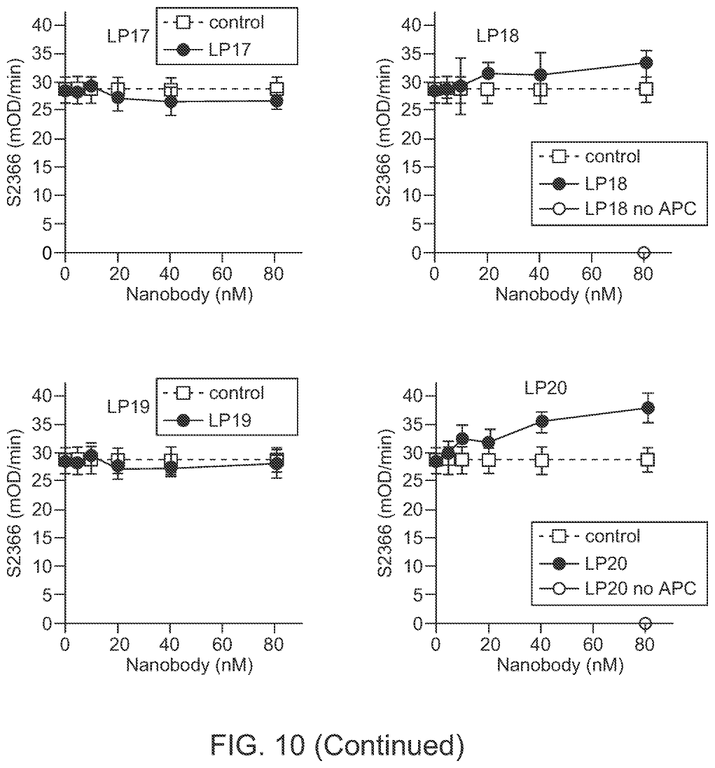

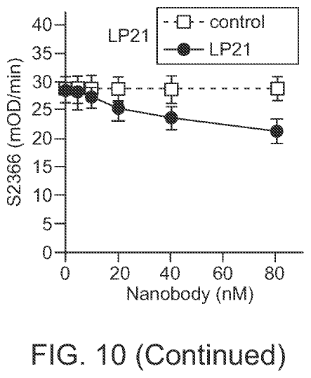

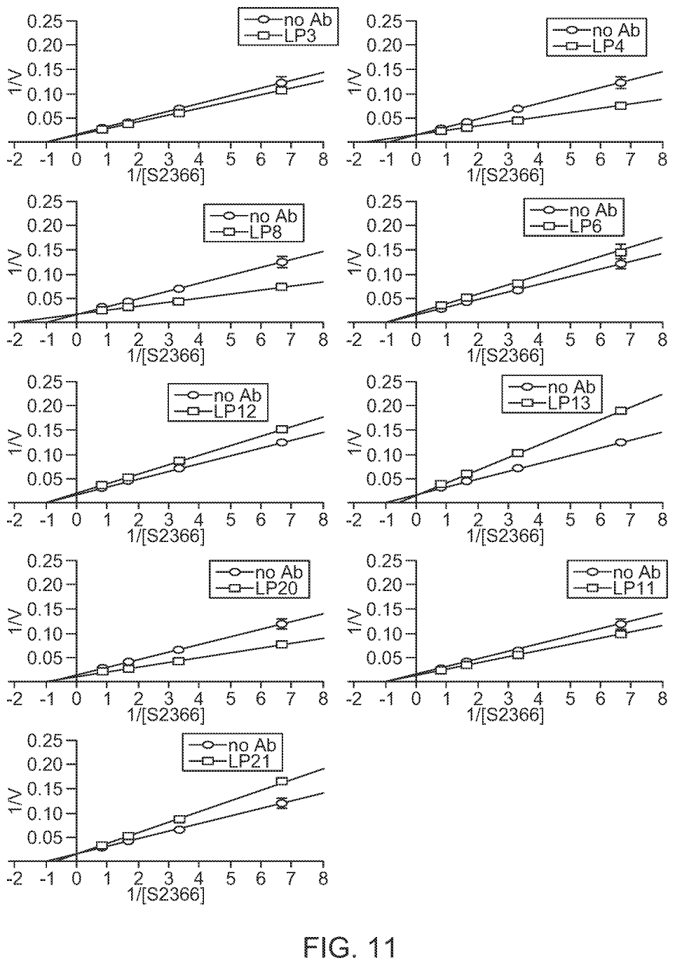

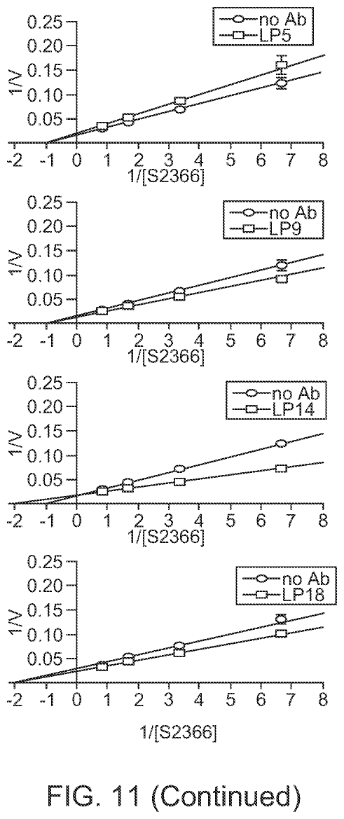

In some embodiments, the antibodies of the present invention inhibit the anticoagulant activity of APC (i.e., relative to the anticoagulant activity in a no-antibody control) and thereby promote blood clot formation. In some embodiments, the antibodies inhibit the anticoagulant activity of APC by at least about 10%, about 20%, about 30%, about 40%, about 50%, about 60%, about 70%, about 80%, about 90%, about 95%, about 96%, about 97%, about 98%, about 99%, or about 100%. The antibodies may exert this effect by blocking APC's ability to inactivate the clotting factors factor Va and factor VIIIa, thereby increasing thrombin generation. Suitable assays for assessing the ability of a particular antibody to inhibit the anticoagulant activity of APC include, without limitation, amidolytic activity assays, substrate cleavage assays (e.g., S-2366), and thrombin-generation assays. Alternatively, the ability of an antibody to decrease clotting time may be measured using an activated partial thromboplastin time (APTT) clotting assay. See Nat Commun 11(1):2992, 2020 for a description of these assays. In the Examples, the ability of 21 anti-APC antibodies to inhibit the anticoagulant activity of APC was tested using both an S-2366 cleavage assay and a Protac®-modified APTT assay (see FIG. 2, FIG. 10, FIG. 11, and Table 6).

For some indications, it may be preferable to use an anti-APC antibody that maintains or enhances the anticoagulant activity of APC. For example, such an antibody may be useful for treating patients with cancer-associated thrombosis, which is a major cause of mortality in cancer patients. Thus, in other embodiments, the antibodies of the present invention enhance or minimally affect the anticoagulant activity of APC.

As used herein, an antibody “minimally affects” a particular APC activity if the level of the APC activity in the presence of the antibody is within +/−20% of the level of the APC activity in a no-antibody control. In some cases, an antibody that “minimally affects” an APC activity is one that has no detectable effect on the APC activity (i.e., one for which the level of the APC activity in the presence of the antibody is the same as the level of the APC activity in a no-antibody control). As used herein, a “no-antibody control” is a comparable sample to which no anti-APC antibody has been added.

For most human therapeutics, it is preferable that an anti-APC antibody maintains or enhances APC's pleiotropic cytoprotective functions. The terms “cytoprotective functions” and “cytoprotective activities” are used interchangeably herein to describe the anti-apoptotic, anti-inflammatory, and endothelial barrier stabilization functions of APC, which all contribute to the regenerative outcomes associated with APC. These functions are often interrelated. For example, cell death (e.g., due to an injury or infection) leads to the release of histones into the extracellular space where they interact with endothelial cells, triggering endothelial cell apoptosis and contributing to systemic inflammation. Thus, the histone cleavage function of APC reduces apoptosis, endothelial barrier disruption, and inflammation.

However, for some indications, it may be preferable to use an anti-APC antibody that blocks APC's cytoprotective functions. For example, a short-term disruption in endothelial barrier function could be used to temporarily increase vascular permeability to induce inflammation for a therapeutic purpose. For cancer treatment, it may be advantageous to block cytoprotective activity to promote an immune response against a tumor.

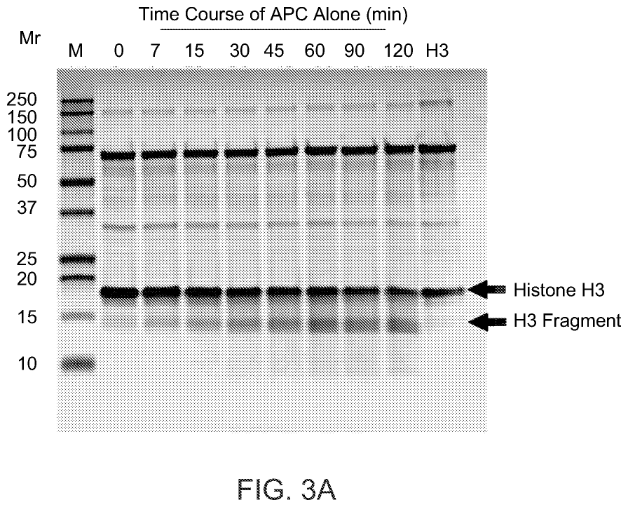

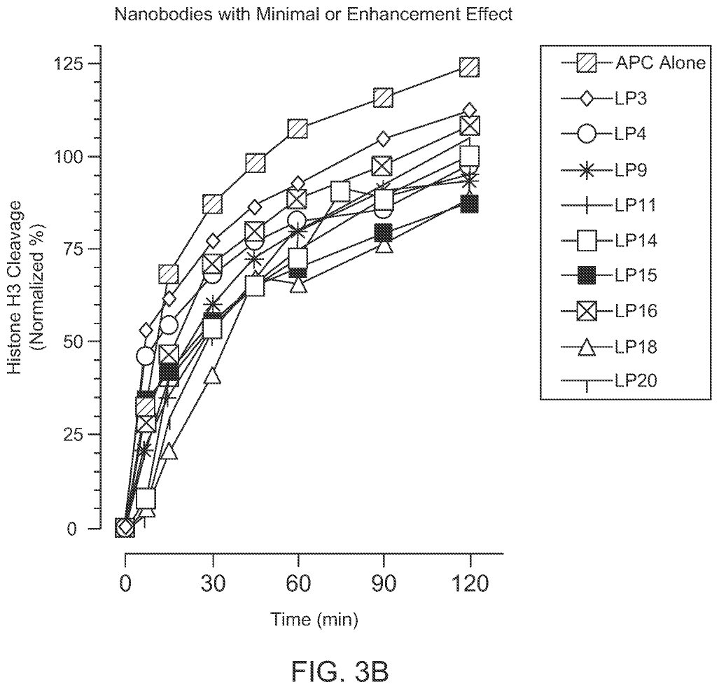

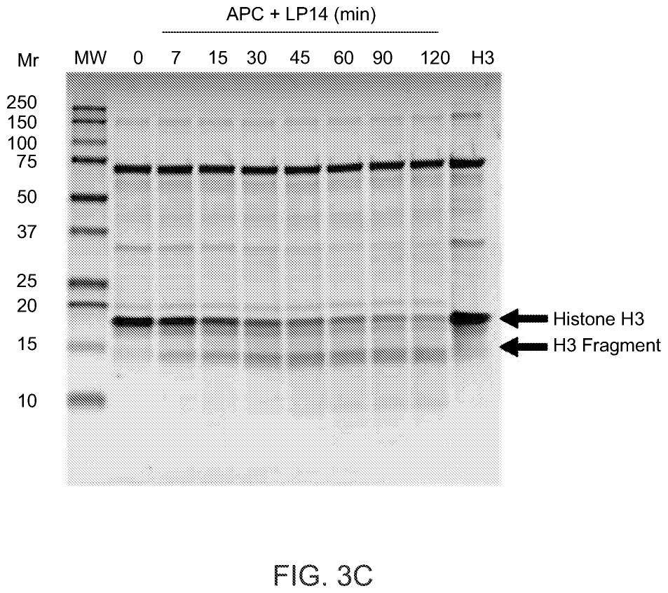

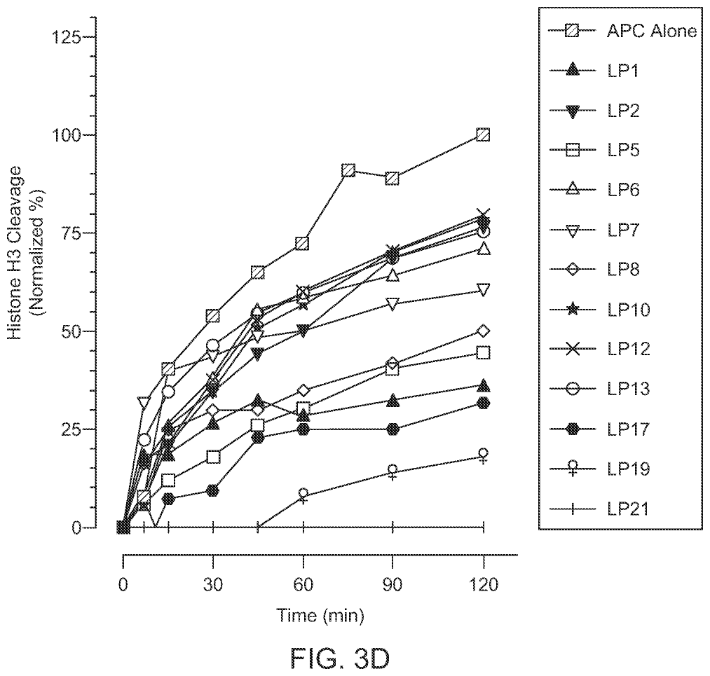

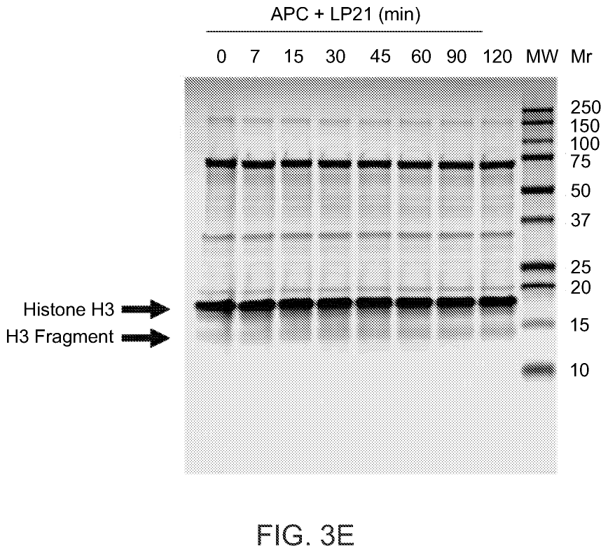

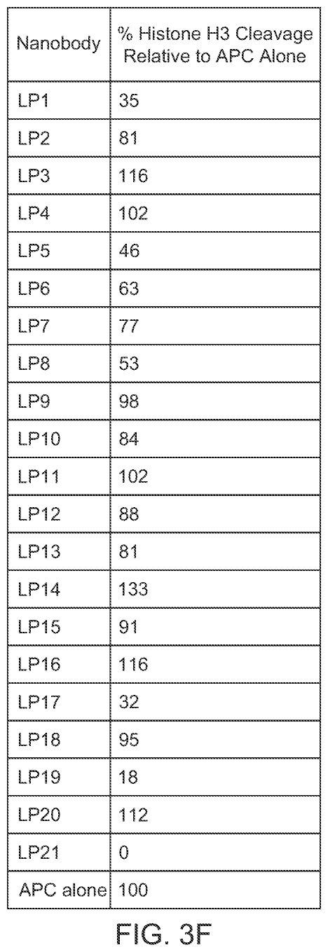

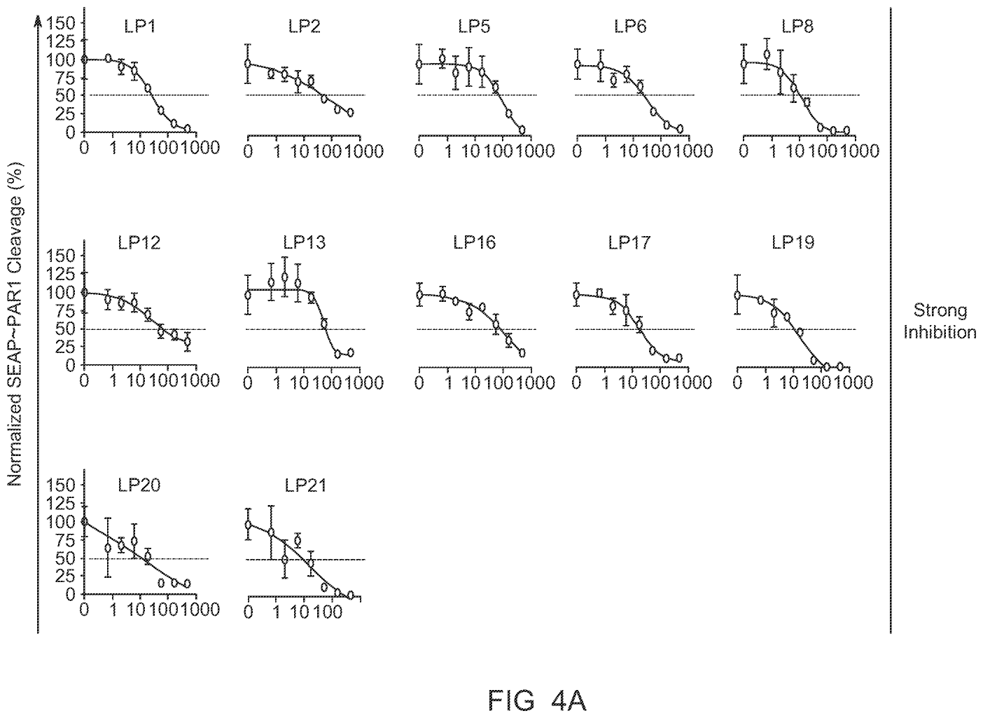

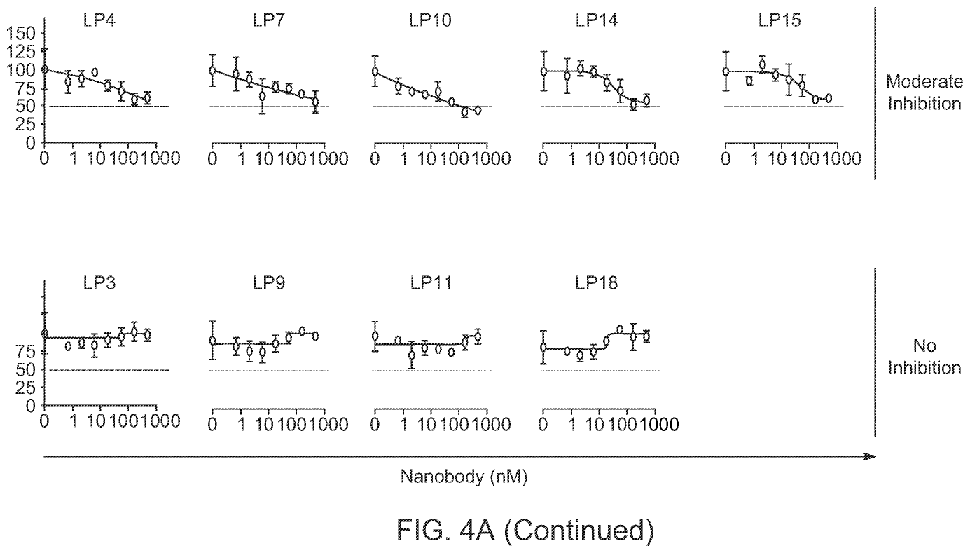

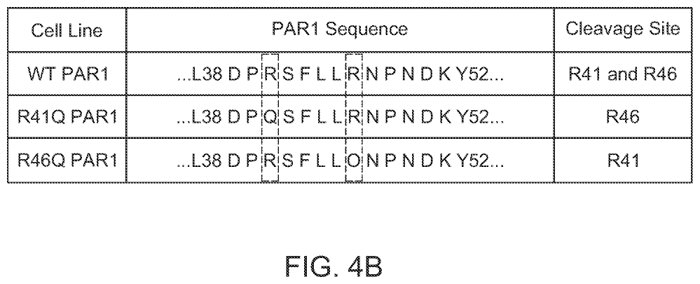

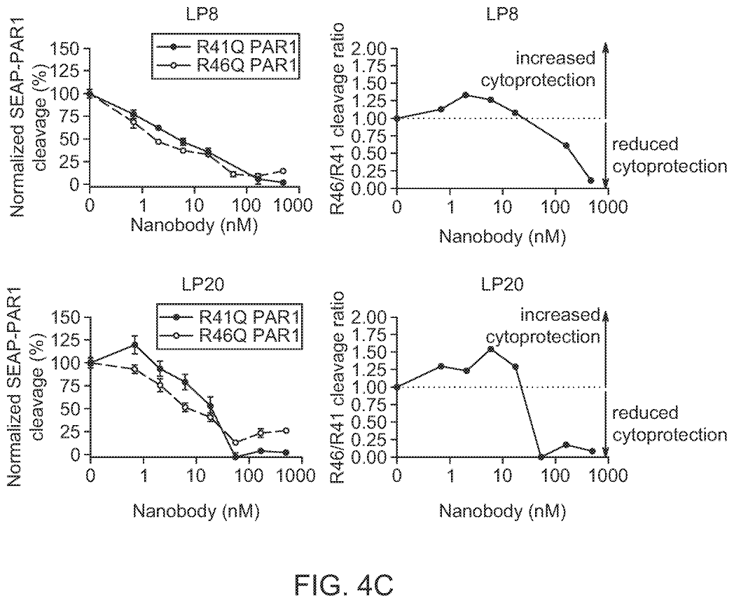

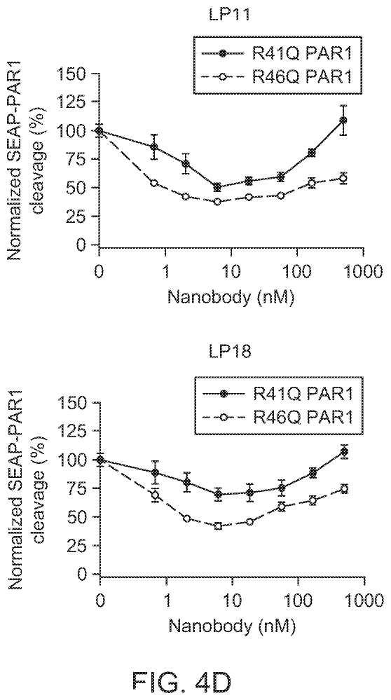

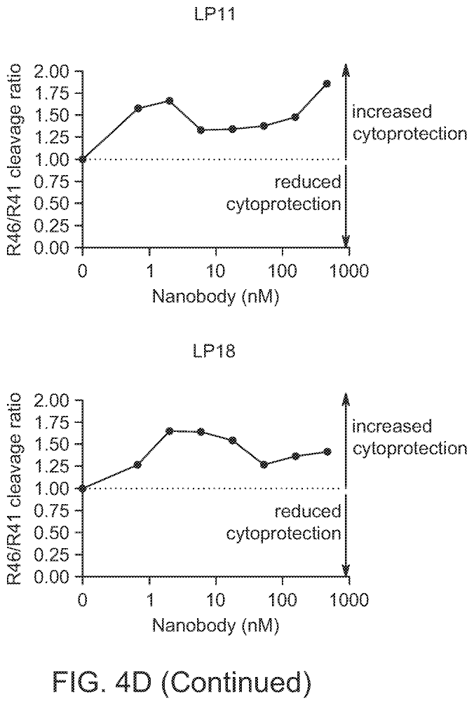

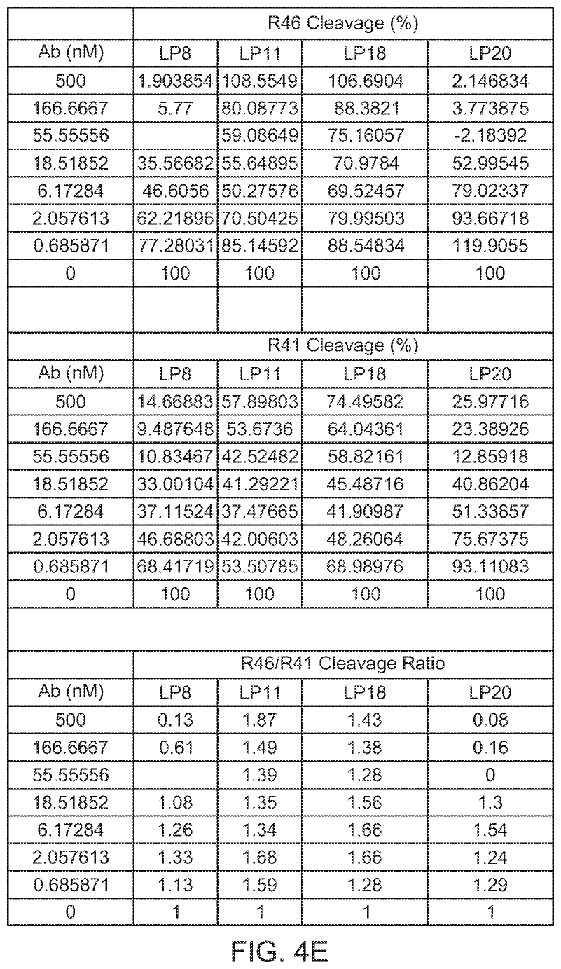

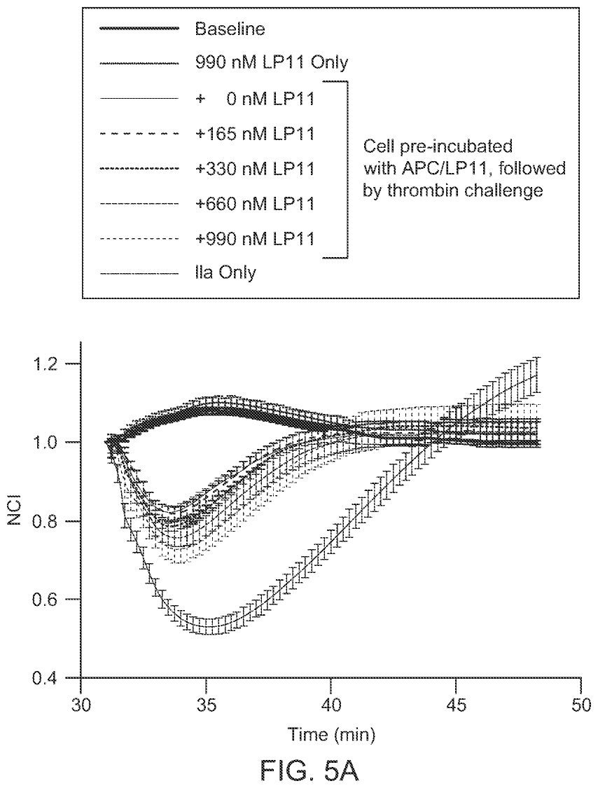

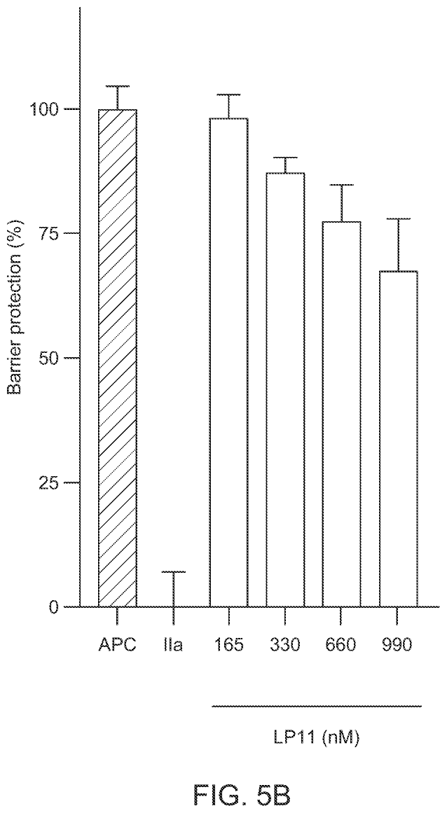

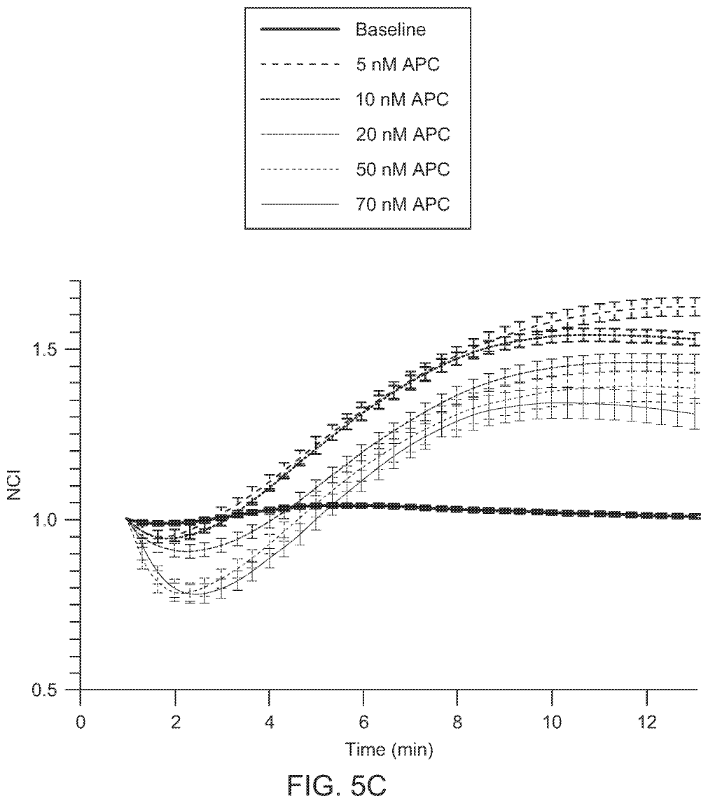

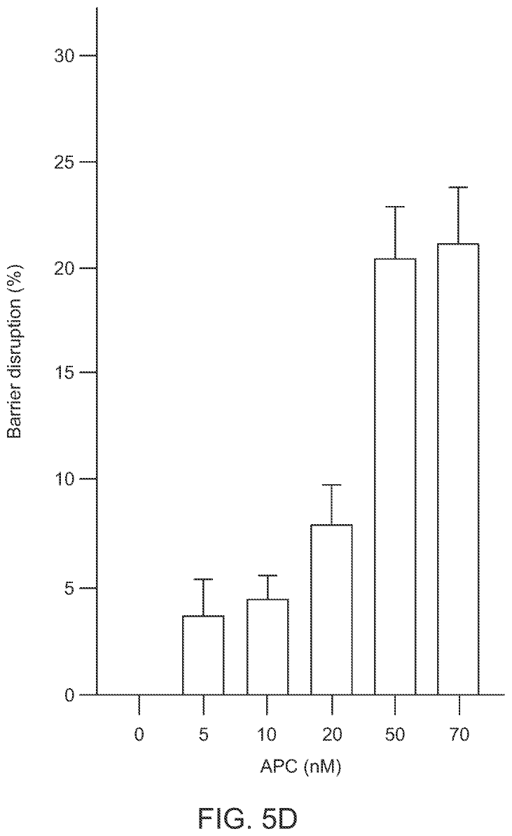

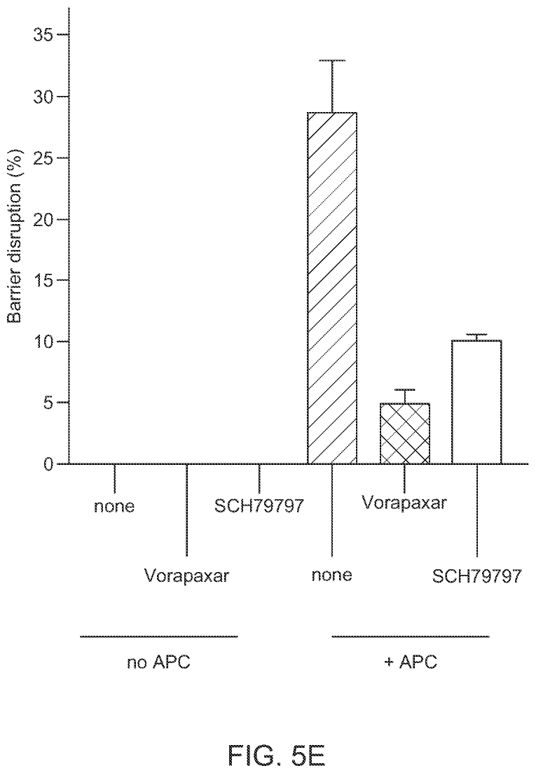

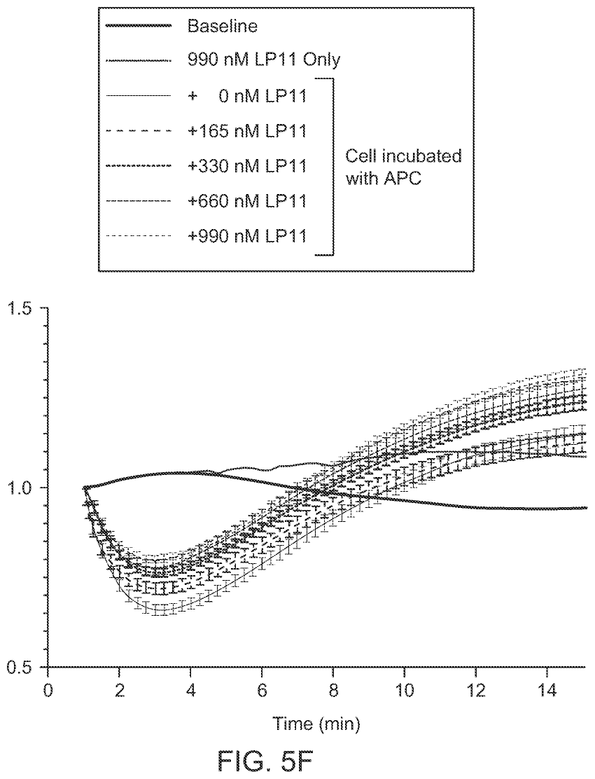

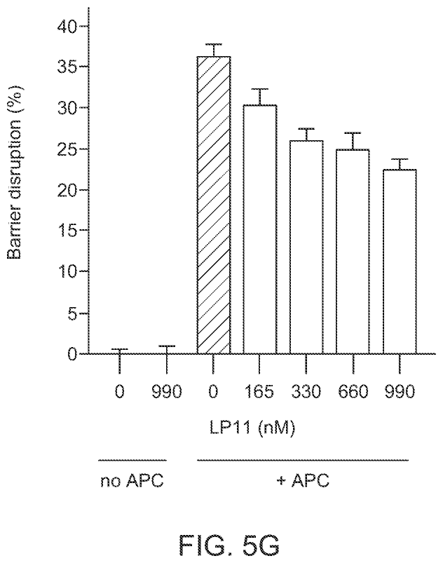

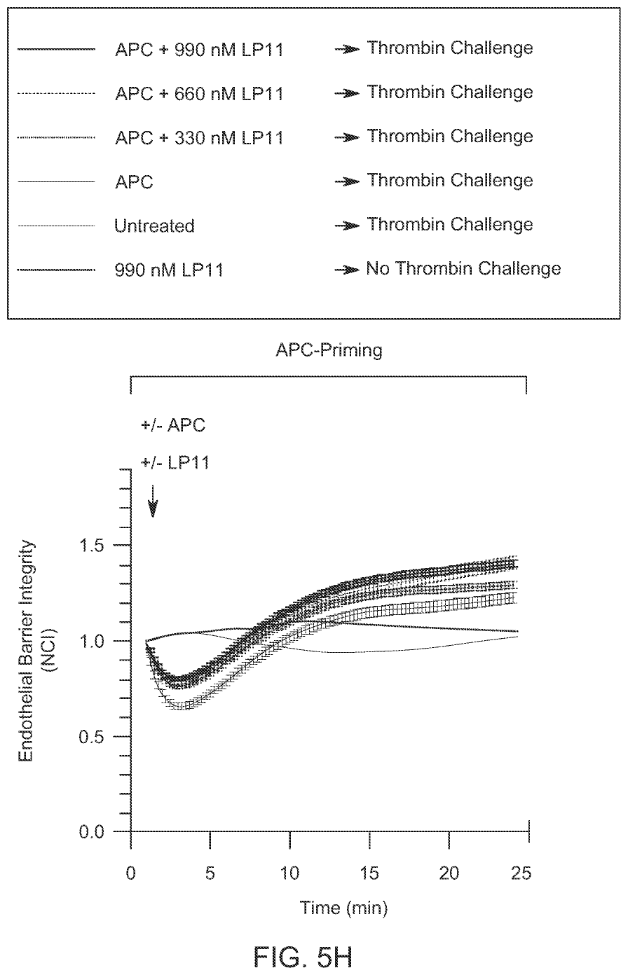

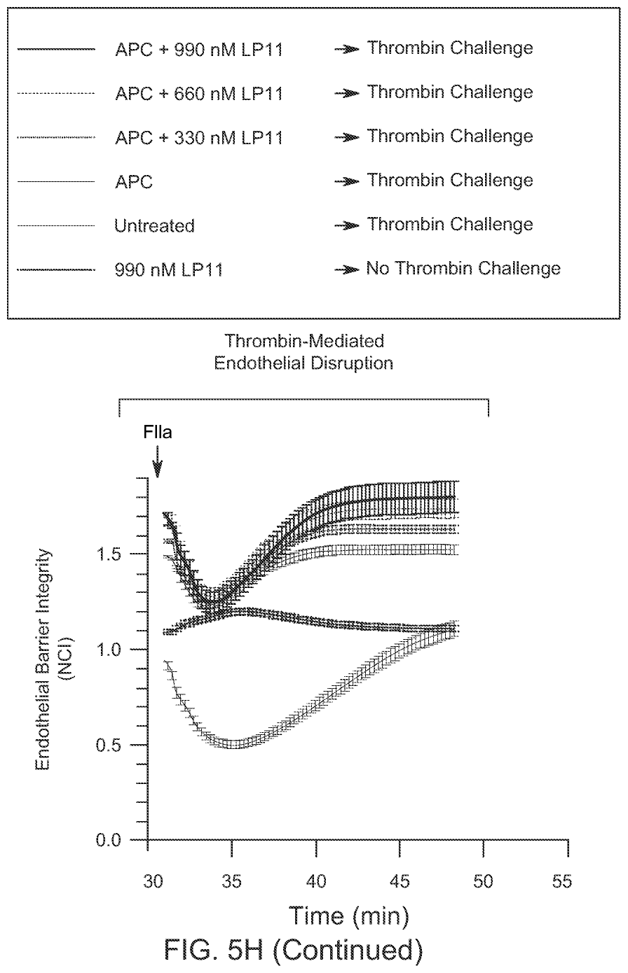

Thus, in some embodiments, the antibodies of the present invention enhance, minimally affect, or inhibit a cytoprotective function of APC (i.e., relative to the cytoprotective function in a no-antibody control). Specifically, in some embodiments, the antibodies (1) enhance, minimally affect, or inhibit APC-mediated histone cleavage, (2) enhance, minimally affect, or inhibit APC-mediated protease-activated receptor 1 (PAR1) cleavage at residue R46, (3) inhibit, minimally affect, or inhibit APC-mediated PAR1 cleavage at residue R41; (4) increase, minimally affect, or decrease the ratio of APC-mediated PAR1 cleavage at residue R46 to APC-mediated PAR1 cleavage at residue R41; and/or (5) enhance, minimally affect, or inhibit APC-mediated endothelial barrier protection. In the Examples, the effects of 21 anti-APC antibodies on APC-mediated histone cleavage were tested using a histone H3 cleavage assay (see FIG. 3). Further, the effects of these 21 antibodies on PAR1 cleavage were tested using a secreted embryonic alkaline phosphatase (SEAP)-PAR1 cleavage assay (see FIG. 4). Using this assay, two antibodies (i.e., LP11 and LP18) were identified that preferentially inhibit APC-mediated cleavage of PAR1 at residue R41 as compared to residue R46, which enhances cytoprotective PAR1 signaling. Finally, the effect of one anti-APC antibody (i.e., LP11) on APC-mediated endothelial barrier protection was tested using an in vitro endothelial barrier function assay (see FIG. 5).

In some embodiments, the antibodies of the present invention increase or decrease the half-life of APC (i.e., relative to the half-life in the absence of the antibody). In other embodiments, the antibodies minimally affect the half-life of APC. In the Examples, the effects of 21 anti-APC antibodies on the half-life of APC were tested using an in vitro plasma half-life assay (see Table 8).

The antibodies disclosed herein bind to exosites (i.e., sites other than the active site) of APC. The antibodies were generated against an active site-blocked APC protein, referred to herein as “APC-PPACK,” which comprises APC bound in its active site by the tripeptide inhibitor Phe-Pro-Arg-chloromethylketone (PPACK). Antibodies that target exosites of APC have been shown to inhibit this enzyme's antithrombotic activity while persevering its beneficial cytoprotective functions (Nat Commun 11(1):2992, 2020). The separation of APC's anticoagulant and cytoprotective functions is possible because these functions involve distinct sites on the protein surface, i.e., the amino acids that mediate APC's interactions with cofactors and substrates are found in exosites that are far removed from the active site.

The antibodies disclosed herein were identified by panning a custom V_{H}H phage library, selecting for antibodies that bind to human APC-PPACK and deselecting for antibodies that bind to human PC and XPPACK. “XPPACK” is another serine kinase that is bound by PPACK. Thus, selection against XPPACK reduces the likelihood that the selected antibodies have affinity for PPACK rather than APC. The V_{H}H phage library was constructed from heavy-chain antibodies generated by immunizing llamas with APC-PPACK. The libraries included only the V_{H}H portion of the resulting llama antibodies, which offer full antigen-binding potential despite being remarkably small (usually 12-14 kDa). The sequences of each V_{H}H antibody and its CDRs were identified via sequencing. The sequence identifiers for the protein sequences of the 158 unique anti-APC antibodies identified in the Examples are listed in Table 1. The sequence identifiers for the DNA sequences of the top 21 antibodies, which were selected for further analysis via functional assays, are listed in Table 2.

TABLE 1
Sequence identifiers for the amino acid sequences of the CDRs
and the full-length V_{H}H region of each of the 158 unique anti-
APC antibodies disclosed herein. The names of the antibodies that
were selected for further analysis in the Examples are highlighted
in bold font and the new names that were given to these antibodies
(i.e., LP1-LP21) are indicated in parentheses.

SEQ ID NO.

| Antibody name | CDR1 | CDR2 | CDR3 | VHH |
|---|---|---|---|---|
| SR16983-R2P1C08 | 1 | 159 | 317 | 475 |
| SR16983-R2P1C10 | 2 | 160 | 318 | 476 |
| SR16983-R2P1C11 | 3 | 161 | 319 | 477 |
| SR16983-R2P1C12 | 4 | 162 | 320 | 478 |
| SR16983-R2P1F04 | 5 | 163 | 321 | 479 |
| SR16983-R3P2A5 | 6 | 164 | 322 | 480 |
| SR16983-R3P2A9 | 7 | 165 | 323 | 481 |
| SR16983-R3P2B6 | 8 | 166 | 324 | 482 |
| SR16983-R3P2D12 | 9 | 167 | 325 | 483 |
| SR16983-R3P2E7 | 10 | 168 | 326 | 484 |
| SR16983-R3P2F4 | 11 | 169 | 327 | 485 |
| SR16983-R3P2G9 | 12 | 170 | 328 | 486 |
| SR16983-R3P2G12 | 13 | 171 | 329 | 487 |
| SR16983-R3P2A07 | 14 | 172 | 330 | 488 |
| SR16983-R3P2A08 | 15 | 173 | 331 | 489 |
| SR16983-R3P2D01 | 16 | 174 | 332 | 490 |
| SR16983-R3P2D02 | 17 | 175 | 333 | 491 |
| SR16983-R3P2D06 | 18 | 176 | 334 | 492 |
| SR16983-R3P2D09 | 19 | 177 | 335 | 493 |
| SR16983-R3P2E05 | 20 | 178 | 336 | 494 |
| SR16983-R3P2G08 | 21 | 179 | 337 | 495 |
| SR16983-R3P2H01 | 22 | 180 | 338 | 496 |
| SR16983-R3P2H05 | 23 | 181 | 339 | 497 |
| SR16983-R3P2H09 | 24 | 182 | 340 | 498 |
| SR16983-R3P2H10 | 25 | 183 | 341 | 499 |
| SR16983-R2-P2-A08 | 26 | 184 | 342 | 500 |
| SR16983-R2-P2-C03 | 27 | 185 | 343 | 501 |
| SR16983-R2-P2-C04 | 28 | 186 | 344 | 502 |
| SR16983-R2-P2-D01 | 29 | 187 | 345 | 503 |
| SR16983-R2-P2-D05 | 30 | 188 | 346 | 504 |
| SR16983-R2-P2-D08 | 31 | 189 | 347 | 505 |
| SR16983-R2-P2-E03 | 32 | 190 | 348 | 506 |
| SR16983-R2-P2-E10 | 33 | 191 | 349 | 507 |
| SR16983-R2-P2-F02 | 34 | 192 | 350 | 508 |
| SR16983-R2-P2-F06 | 35 | 193 | 351 | 509 |
| SR16983-R2-P2-F07 | 36 | 194 | 352 | 510 |
| SR16983-R2-P2-G01 | 37 | 195 | 353 | 511 |
| SR16983-R2-P2-G09 | 38 | 196 | 354 | 512 |
| SR16983-R2-P2-G11 | 39 | 197 | 355 | 513 |
| SR16983-R2-P2-H04 | 40 | 198 | 356 | 514 |
| SR16983-R2-P3-A04 | 41 | 199 | 357 | 515 |
| SR16983-R2-P3-A12 | 42 | 200 | 358 | 516 |
| SR16983-R2-P3-B01 | 43 | 201 | 359 | 517 |
| SR16983-R2-P3-B05 | 44 | 202 | 360 | 518 |
| SR16983-R2-P3-D06 | 45 | 203 | 361 | 519 |
| (LP1) | | | | |
| SR16983-R2-P3-D08 | 46 | 204 | 362 | 520 |
| SR16983-R2-P3-E04 | 47 | 205 | 363 | 521 |
| SR16983-R2-P3-E05 | 48 | 206 | 364 | 522 |
| SR16983-R2-P3-E06 | 49 | 207 | 365 | 523 |
| SR16983-R2-P3-F01 | 50 | 208 | 366 | 524 |
| SR16983-R2-P3-F07 | 51 | 209 | 367 | 525 |
| SR16983-R2-P3-F10 | 52 | 210 | 368 | 526 |
| SR16983-R2-P3-F12 | 53 | 211 | 369 | 527 |
| SR16983-R2-P3-H10 | 54 | 212 | 370 | 528 |
| (LP2) | | | | |
| SR16983-R2-P4-B05 | 55 | 213 | 371 | 529 |
| SR16983-R2-P4-C02 | 56 | 214 | 372 | 530 |
| SR16983-R2-P4-D07 | 57 | 215 | 373 | 531 |
| SR16983-R2-P4-E03 | 58 | 216 | 374 | 532 |
| SR16983-R2-P4-E06 | 59 | 217 | 375 | 533 |
| SR16983-R2-P4-E08 | 60 | 218 | 376 | 534 |
| SR16983-R2-P4-E09 | 61 | 219 | 377 | 535 |
| (LP3) | | | | |
| SR16983-R2-P4-E11 | 62 | 220 | 378 | 536 |
| SR16983-R2-P4-F01 | 63 | 221 | 379 | 537 |
| SR16983-R2-P4-F03 | 64 | 222 | 380 | 538 |
| SR16983-R2-P4-F09 | 65 | 223 | 381 | 539 |
| SR16983-R2-P4-G01 | 66 | 224 | 382 | 540 |
| SR16983-R2-P4-G02 | 67 | 225 | 383 | 541 |
| SR16983-R2-P4-G06 | 68 | 226 | 384 | 542 |
| SR16983-R2-P4-G08 | 69 | 227 | 385 | 543 |
| SR16983-R2-P4-H08 | 70 | 228 | 386 | 544 |
| SR16983-R2-P5-A12 | 71 | 229 | 387 | 545 |
| SR16983-R2-P5-D01 | 72 | 230 | 388 | 546 |
| SR16983-R2-P5-H07 | 73 | 231 | 389 | 547 |
| SR16983-R3-P2-A06 | 74 | 232 | 390 | 548 |
| (LP4) | | | | |
| SR16983-R3-P2-A08 | 75 | 233 | 391 | 549 |
| SR16983-R3-P2-B02 | 76 | 234 | 392 | 550 |
| SR16983-R3-P2-B05 | 77 | 235 | 393 | 55 |
| SR16983-R3-P2-C08 | 78 | 236 | 394 | 552 |
| SR16983-R3-P2-D01 | 79 | 237 | 395 | 553 |
| SR16983-R3-P2-D12 | 80 | 238 | 396 | 554 |
| SR16983-R3-P2-E09 | 81 | 239 | 397 | 555 |
| SR16983-R3-P2-F04 | 82 | 240 | 398 | 556 |
| (LP5) | | | | |
| SR16983-R3-P2-F07 | 83 | 241 | 399 | 557 |
| SR16983-R3-P2-F11 | 84 | 242 | 400 | 558 |
| (LP6) | | | | |
| SR16983-R3-P2-G01 | 85 | 243 | 401 | 559 |
| SR16983-R3-P2-G05 | 86 | 244 | 402 | 560 |
| SR16983-R3-P2-H03 | 87 | 245 | 403 | 561 |
| SR16983-R3-P2-H08 | 88 | 246 | 404 | 562 |
| (LP7) | | | | |
| SR16983-R3-P3-A02 | 89 | 247 | 405 | 563 |
| SR16983-R3-P3-A04 | 90 | 248 | 406 | 564 |
| (LP8) | | | | |
| SR16983-R3-P3-A08 | 91 | 249 | 407 | 565 |
| SR16983-R3-P3-A12 | 92 | 250 | 408 | 566 |
| SR16983-R3-P3-B03 | 93 | 251 | 409 | 567 |
| SR16983-R3-P3-B04 | 94 | 252 | 410 | 568 |
| SR16983-R3-P3-B05 | 95 | 253 | 411 | 569 |
| (LP9) | | | | |
| SR16983-R3-P3-C06 | 96 | 254 | 412 | 570 |
| SR16983-R3-P3-C10 | 97 | 255 | 413 | 571 |
| SR16983-R3-P3-D03 | 98 | 256 | 414 | 572 |
| SR16983-R3-P3-E02 | 99 | 257 | 415 | 573 |
| SR16983-R3-P3-E04 | 100 | 258 | 416 | 574 |
| SR16983-R3-P3-E06 | 101 | 259 | 417 | 575 |
| (LP10) | | | | |
| SR16983-R3-P3-F01 | 102 | 260 | 418 | 576 |
| SR16983-R3-P3-F03 | 103 | 261 | 419 | 577 |
| (LP11) | | | | |
| SR16983-R3-P3-F04 | 104 | 262 | 420 | 578 |
| SR16983-R3-P3-F06 | 105 | 263 | 421 | 579 |
| (LP12) | | | | |
| SR16983-R3-P3-G03 | 106 | 264 | 422 | 580 |
| SR16983-R3-P3-G04 | 107 | 265 | 423 | 581 |
| SR16983-R3-P3-G05 | 108 | 266 | 424 | 582 |
| SR16983-R3-P3-G08 | 109 | 267 | 425 | 583 |
| (LP13) | | | | |
| SR16983-R3-P3-H05 | 110 | 268 | 426 | 584 |
| SR16983-R3-P3-H06 | 111 | 269 | 427 | 585 |
| SR16983-R3-P3-H10 | 112 | 270 | 428 | 586 |
| SR16983-R3-P3-H11 | 113 | 271 | 429 | 587 |
| SR16983-R3-P4-A02 | 114 | 272 | 430 | 588 |
| SR16983-R3-P4-A05 | 115 | 273 | 431 | 589 |
| SR16983-R3-P4-A06 | 116 | 274 | 432 | 590 |
| (LP14) | | | | |
| SR16983-R3-P4-A07 | 117 | 275 | 433 | 591 |
| SR16983-R3-P4-A08 | 118 | 276 | 434 | 592 |
| SR16983-R3-P4-C02 | 119 | 277 | 435 | 593 |
| SR16983-R3-P4-E04 | 120 | 278 | 436 | 594 |
| SR16983-R3-P4-E06 | 121 | 279 | 437 | 595 |
| SR16983-R3-P4-E11 | 122 | 280 | 438 | 596 |
| SR16983-R3-P4-F06 | 123 | 281 | 439 | 597 |
| SR16983-R3-P4-G01 | 124 | 282 | 440 | 598 |
| SR16983-R3-P4-G09 | 125 | 283 | 441 | 599 |
| SR16983-R3-P4-H10 | 126 | 284 | 442 | 600 |
| SR16983-R3-P5-A06 | 127 | 285 | 443 | 601 |
| SR16983-R3-P5-A11 | 128 | 286 | 444 | 602 |
| SR16983-R3-P5-B01 | 129 | 287 | 445 | 603 |
| SR16983-R3-P5-B02 | 130 | 288 | 446 | 604 |
| SR16983-R3-P5-C03 | 131 | 289 | 447 | 605 |
| SR16983-R3-P5-C09 | 132 | 290 | 448 | 606 |
| SR16983-R3-P5-D01 | 133 | 291 | 449 | 607 |
| SR16983-R3-P5-D08 | 134 | 292 | 450 | 608 |
| SR16983-R3-P5-E10 | 135 | 293 | 451 | 609 |
| SR16983-R3-P5-E11 | 136 | 294 | 452 | 610 |
| SR16983-R3-P5-F02 | 137 | 295 | 453 | 611 |
| SR16983-R3-P5-F03 | 138 | 296 | 454 | 612 |
| SR16983-R3-P5-F04 | 139 | 297 | 455 | 613 |
| SR16983-R3-P5-G07 | 140 | 298 | 456 | 614 |
| SR16983-R3-P5-G12 | 141 | 299 | 457 | 615 |
| SR16983-R3-P5-H05 | 142 | 300 | 458 | 616 |
| (LP15) | | | | |
| SR16983-R3-P5-H06 | 143 | 301 | 459 | 617 |
| SR16983-R4-P1-A04 | 144 | 302 | 460 | 618 |
| (LP16) | | | | |
| SR16983-R4-P1-A05 | 145 | 303 | 461 | 619 |
| SR16983-R4-P1-A09 | 146 | 304 | 462 | 620 |
| (LP17) | | | | |
| SR16983-R4-P1-A11 | 147 | 305 | 463 | 621 |
| (LP18) | | | | |
| SR16983-R4-P1-A12 | 148 | 306 | 464 | 622 |
| SR16983-R4-P1-B06 | 149 | 307 | 465 | 623 |
| (LP19) | | | | |
| SR16983-R4-P1-B07 | 150 | 308 | 466 | 624 |
| (LP20) | | | | |
| SR16983-R4-P1-B11 | 151 | 309 | 467 | 625 |
| SR16983-R4-P1-C01 | 152 | 310 | 468 | 626 |
| SR16983-R4-P1-C05 | 153 | 311 | 469 | 627 |
| SR16983-R4-P1-C07 | 154 | 312 | 470 | 628 |
| SR16983-R4-P1-C10 | 155 | 313 | 471 | 629 |
| SR16983-R4-P1-C12 | 156 | 314 | 472 | 630 |
| SR16983-R4-P1-D01 | 157 | 315 | 473 | 631 |
| (LP21) | | | | |
| SR16983-R4-P1-D04 | 158 | 316 | 474 | 632 |

TABLE 2
Sequence identifiers for the DNA sequences of the CDRs and
the full-length V_{H}H region of the 21 antibodies that were
selected for further analysis in functional assays.

SEQ ID NO.

| Antibody name | CDR1 | CDR2 | CDR3 | VHH |
|---|---|---|---|---|
| LP1 | 635 | 656 | 677 | 698 |
| LP2 | 636 | 657 | 678 | 699 |
| LP3 | 637 | 658 | 679 | 700 |
| LP4 | 638 | 659 | 680 | 701 |
| LP5 | 639 | 660 | 681 | 702 |
| LP6 | 640 | 661 | 682 | 703 |
| LP7 | 641 | 662 | 683 | 704 |
| LP8 | 642 | 663 | 684 | 705 |
| LP9 | 643 | 664 | 685 | 706 |
| LP10 | 644 | 665 | 686 | 707 |
| LP11 | 645 | 666 | 687 | 708 |
| LP12 | 646 | 667 | 688 | 709 |
| LP13 | 647 | 668 | 689 | 710 |
| LP14 | 648 | 669 | 690 | 711 |
| LP15 | 649 | 670 | 691 | 712 |
| LP16 | 650 | 671 | 692 | 713 |
| LP17 | 651 | 672 | 693 | 714 |
| LP18 | 652 | 673 | 694 | 715 |
| LP19 | 653 | 674 | 695 | 716 |
| LP20 | 654 | 675 | 696 | 717 |
| LP21 | 655 | 676 | 697 | 718 |

The antibodies of the present invention are “isolated,” meaning that they are substantially free of other biological molecules, including antibodies having different antigenic specificities and other cellular materials. In some embodiments, the isolated antibodies are at least about 75%, about 80%, about 90%, about 95%, about 97%, about 99%, about 99.9% or about 100% pure by dry weight. Purity may be measured using standard method such as column chromatography, polyacrylamide gel electrophoresis, or high-performance liquid chromatography (HPLC). An isolated antibody that binds to human APC can, however, have cross-reactivity to other closely related antigens, e.g., APC homologs from other species.

The term “complementarity-determining regions” or “CDRs” refers to hypervariable regions that together form an antigen-binding surface that is complementary to the three-dimensional structure of the antigen. In conventional antibodies, each V_{H} and V_{L} comprises three complementarity-determining regions. The CDRs are numbered as “CDR1,” “CDR2,” and “CDR3 starting from the N-terminus of the V_{H} or V_{L} (see Proc Natl Acad Sci USA 72(12):5107, 1975; J Exp Med 132(2):211, 1970). Thus, in conventional antibodies (i.e., antibodies that comprise two heavy chains and two light chains), an antigen-binding site includes six CDRs: the three CDRs of the V_{H} and the three CDRs of the V_{L}. However, in a heavy-chain antibody, the antigen-binding site includes only the three CDRs of the V_{H} (FIG. 15).

Because CDR sequences are responsible for most antibody-antigen interactions, it is possible to create recombinant antibodies that mimic the properties of a specific naturally occurring antibody by grafting the CDR sequences of the naturally occurring antibody into the framework sequences of a different antibody with different properties (see, e.g., Nature 332:323-327, 1998; Nature 321:522-525, 1986; Proc Natl Acad Sci USA 86:10029-10033, 1989). Such framework sequences can be obtained from public databases that include germline antibody gene sequences. Thus, in some embodiments, the CDRs of the antibodies described herein are grafted into another antibody framework.

The CDRs disclosed herein as SEQ ID NOs: 1-474 are the CDRs of camelid-derived V_{H}H antibodies that were selected in the screen described in the Examples. Of the three CDRs, CDR3 is believed to be the main contributor for antigen recognition and specificity, and CDR1 and CDR2 are believed to contribute to binding strength (J Mol Biol 430:4369, 2018; Protein Eng Des Sel 31:267, 2018; Proteins 86:697, 2018). Thus, in some embodiments the antibodies of the present invention comprise a CDR3 of a V_{H}H antibody disclosed herein. Specifically, in some embodiments, the antibodies of the present invention comprise a CDR3 comprising or consisting of an amino acid sequence selected from the group consisting of SEQ ID NOs: 317-474.

In other embodiments, the antibodies of the present invention further comprise a CDR1 and a CDR2 of a V_{H}H antibody disclosed herein. Specifically, these antibodies further comprise (a) a CDR1 comprising or consisting of an amino acid sequence selected from the group consisting of SEQ ID NOs: 1-158, (b) a CDR2 comprising or consisting of an amino acid sequence selected from the group consisting of SEQ ID NOs: 159-316, or (c) both a CDR1 comprising or consisting of an amino acid sequence selected from the group consisting of SEQ ID NOs: 1-158 and a CDR2 comprising or consisting of an amino acid sequence selected from the group consisting of SEQ ID NOs: 159-316.

In certain embodiments, the antibodies of the present invention comprise a CDR1, CDR2, and CDR3 that are all derived from a single V_{H}H antibody disclosed herein. In other words, these antibodies comprise the paratope (i.e., a set of three CDRs that form an antigen-binding region) of a V_{H}H antibody disclosed herein. Specifically, these antibodies comprise the CDR1, CDR2, and CDR3 of a V_{H}H antibody having an amino acid sequence selected from the group consisting of SEQ ID NOs: 475-632.

Additionally, the present invention provides antibodies that comprise or consist of a V_{H}H disclosed herein. Specifically, these antibodies comprise or consist of a V_{H}H selected from the group consisting of SEQ ID NOs: 475-632.

In some embodiments, the antibodies comprise one or more amino acid modifications. As used herein, the term “amino acid modification” refers to a change in a polypeptide sequence. Amino acid modifications include deletions, additions, and substitutions of one or more amino acid residues. The antibodies of the present invention may comprise any combination of amino acid modifications so long as they retain the ability to bind APC with minimal to no binding to PC. In some embodiments, the antibody comprises 1, 2, 3, 4, 5, 6, 7, 8, 9, 10, 11, 12, 13, 14, 15, 16, 17, 18, 19, or 20 or more amino acid modifications. For example, in some embodiments, the antibody comprises a variant of a V_{H}H selected from SEQ ID NOs: 475-632 comprising 1, 2, 3, 4, 5, 6, 7, 8, 9, 10, 11, 12, 13, 14, 15, 16, 17, 18, 19, or 20 amino acid modifications relative to the parent antibody. In other embodiments, the antibody comprises a variant of a CDR3 selected from SEQ ID NOS: 317-474 that comprises at least 1 amino acid modification.

In some embodiments, one or more of the amino acid modifications are conservative substitutions. As used herein, the term “conservative substitution” refers to an amino acid substitution that substantially conserves the structure and the function of the native polypeptide. Specifically, conservative substitutions generally maintain (a) the structure of the polypeptide backbone around the substitution (e.g., as a beta sheet or alpha helix), (b) the charge or hydrophobicity of the molecule at the site of the substitution, and/or (c) the bulk of the side chain. Examples of conservative substitutions are shown in Table 3.

TABLE 3
List of conservative amino acid substitutions

| Original reside | Conservative substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

In some embodiments, the antibodies of the present invention comprise or consists of a V_{H}H that has at least 99, 98, 97, 96, 95, 94, 93, 92, 91, 90, 85, 80, or 75 percent sequence identity to one of the amino acid sequences of SEQ ID NOs: 475-632. In some embodiments, the antibodies comprise a CDR3 that has at least 99, 98, 97, 96, 95, 94, 93, 92, 91, or 90 percent sequence identity to one of the amino acid sequences of SEQ ID NOs: 317-474.

“Percentage of sequence identity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100. Protein and nucleic acid sequence identities can be determined using the Basic Local Alignment Search Tool (“BLAST”), which is well known in the art (Proc. Natl. Acad. Sci. USA (1990) 87: 2267-2268; Nucl. Acids Res. (1997) 25: 3389-3402). The BLAST programs identify homologous sequences by identifying similar segments, which are referred to herein as “high-scoring segment pairs”, between a query amino acid or nucleic acid sequence and a test sequence which is preferably obtained from a protein or nucleic acid sequence database. Preferably, the statistical significance of a high-scoring segment pair is evaluated using the statistical significance formula (Proc. Natl. Acad. Sci. USA (1990) 87: 2267-2268). The BLAST programs can be used with the default parameters or with modified parameters provided by the user.

The present invention further provides antibodies that compete with an antibody described herein for binding to APC. Like the other antibodies described herein, these antibodies specifically bind to APC and minimally bind to PC. An antibody is said to “compete” with the binding of another antibody for a particular epitope if binding of one antibody results in decreased binding of the other antibody. Competition can occur either because the antibodies bind to the same epitope, or because the binding of one antibody interferes sterically with the binding of the other antibody or causes a confirmational change that interferes with the binding of the other antibody. In some cases, a first antibody can inhibit the binding of a second antibody to its epitope without the second antibody inhibiting the binding of the first antibody to its epitope. However, in cases where both the first and the second antibody detectably inhibit the binding of the other antibody (whether to the same, greater, or lesser extent) the antibodies are said to “cross-compete” with each other for binding of their epitope(s). Antibodies that compete with or cross-compete with an antibody described herein for binding to APC are encompassed by the present invention.

### Nucleic Acids, Vectors, and Host Cells:

An antibody of the present invention can be produced by introducing a nucleic acid encoding the antibody into a host cell and providing suitable conditions for protein expression. Thus, in a second aspect, the present invention provides nucleic acids encoding the antibodies disclosed herein. Specifically, the invention includes nucleic acids encoding (1) an antibody comprising a V_{H}H comprising or consisting of an amino acid sequence selected from SEQ ID NOs: 475-632, (2) an antibody comprising a CDR3 comprising or consisting of an amino acid sequence selected from SEQ ID NOs: 317-474, or (3) any other antibody described herein. In some embodiments, the nucleic acids comprise (1) a nucleic acid that encodes a CDR1 and is selected from SEQ ID NOs: 635-655, (2) a nucleic acid that encodes a CDR2 and is selected from SEQ ID NOs: 656-676, and/or (3) a nucleic acid that encodes a CDR3 and is selected from SEQ ID NOs: 677-697. In some embodiments, the nucleic acids encode a V_{H}H selected from SEQ ID NOs: 698-718.

The terms “nucleic acid,” “polynucleotide,” and “oligonucleotide” are used interchangeably to refer to a polymer of DNA or RNA, which may be single-stranded or double-stranded, synthesized, or obtained (e.g., isolated and/or purified) from natural sources. Nucleic acids may contain natural, non-natural, or altered nucleotides, and may contain natural, non-natural, or altered internucleotide linkages (e.g., phosphoroamidate or phosphorothioate linkages). In some embodiments, the nucleic acids of the present invention are “isolated,” meaning that they are separated away from other cellular materials.

In a third aspect, the present invention provides vectors comprising the nucleic acids disclosed herein. The term “vector” refers to a DNA molecule that is used to carry a particular DNA segment (i.e., a DNA segment included in the vector) into a host cell. Some vectors are capable of autonomous replication in a host cell (e.g., bacterial vectors that include an origin of replication and episomal mammalian vectors). Other vectors can be integrated into the genome of a host cell such that they are replicated along with the host genome (e.g., viral vectors and transposons). Vectors may include heterologous genetic elements that are necessary for propagation of the vector or for expression of an encoded gene product (e.g., a promoter). Vectors may also include a reporter gene and/or a selectable marker gene. Suitable vectors include plasmids (i.e., circular double-stranded DNA molecules) and mini-chromosomes. Vectors suitable for use with the present invention comprise a DNA segment encoding an antibody described herein and a heterogeneous sequence that allows for expression of the encoded antibody.

In a fourth aspect, the present invention provides host cells comprising the nucleic acids and vectors disclosed herein. The term “host cell” is meant to refer to a transgenic cell in which heterologous DNA can be expressed. The nucleic acids or vectors disclosed herein may be introduced into a host cell using standard techniques including, for example, electroporation, heat shock, lipofection, microinjection, and particle bombardment. It is generally advantageous to express antibodies in eukaryotic host cells to ensure that they are properly modified, folded, and secreted. However, because the V_{H}H antibodies of the present invention have a monomeric structure and lack post-translational modifications, they can readily be produced by prokaryotic cells, such as Escherichia coli, Saccharomyces cerevisiae, and Pichia pastoris.

In a fifth aspect, the present invention also provides methods of producing an antibody using the host cells disclosed herein. The methods comprise: (a) culturing a host cell disclosed herein under conditions that result in production of the antibody, and (b) isolating the antibody from the host cell. In these methods, antibodies are produced by culturing host cells for a sufficient period of time to allow for expression of the antibody in the host cells. Antibodies can then be recovered from the cell culture using standard protein purification methods, such as ultrafiltration, affinity chromatography, size exclusion chromatography, ion exchange chromatography, and centrifugation. Methods for expressing and purifying proteins are well known in the art (see, e.g., Nat Methods 5(2): 135-146, 2008).

### Pharmaceutical Compositions:

In sixth aspect, the present invention provides pharmaceutical compositions comprising a therapeutically effective amount of an antibody disclosed herein and a pharmaceutically acceptable carrier. “Pharmaceutically acceptable” carriers are known in the art and include, but are not limited to, diluents, preservatives, solubilizers, emulsifiers, liposomes, nanoparticles, and adjuvants. Pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, and suspensions, including saline and buffered media.

The compositions of the present invention may further include diluents of various pH, ionic strength, and buffer content (e.g., Tris-HCl, acetate, phosphate), additives to prevent absorption to surfaces, detergents (e.g., Tween 20, Tween 80, Pluronic F68), solubilizing agents (e.g., glycerol, polyethylene glycerol), antioxidants (e.g., ascorbic acid, sodium metabisulfite, L-methionine), bulking substances, or tonicity modifiers (e.g., sucrose, mannitol). Within the compositions, the antibodies may be covalently attached polymers (e.g., polyethylene glycol), complexed with metal ions, or incorporated into or onto particulate preparations of polymeric compounds (e.g., polylactic acid, polyglycolic acid, hydrogels) or onto liposomes, microemulsions, micelles, multilamellar vesicles, erythrocyte ghosts, or spheroplasts. Inclusion of such compounds in the compositions will influence the physical state, solubility, stability, rate of in vivo release, and rate of in vivo clearance. The compositions may also be formulated in lipophilic depots (e.g., fatty acids, waxes, oils) for controlled or sustained release.

### Methods:

In a seventh aspect, the present invention provides methods for treating or preventing a condition. The methods comprise administering a therapeutically effective amount of an antibody or pharmaceutical composition disclosed herein to a subject.

The antibodies of the present invention can, optionally, be administered in combination with an exogenous APC protein to confer additional properties that cannot be achieved via antibody binding to the endogenous APC protein. Thus, in some embodiments, the methods comprise administering an exogenous APC protein or a variant thereof (e.g., 3K3A-APC) that is specifically bound to one or more of the antibodies disclosed herein to a subject.

As used herein, the term “treating” describes the management and care of a patient for the purpose of combating a condition. Treating includes the administration of an antibody or pharmaceutical composition of the present invention to alleviate the symptoms or complications of the condition or to eliminate the condition. As used herein, the term “condition” is used to refer to a health problem with certain characteristics and/or symptoms. The term condition is meant to encompass diseases, disorders, syndromes, and the like.

As used herein, the term “preventing” describes the management and care of a patient for the purpose of preventing the onset of symptoms or complications of a condition.

As used herein, the term “administering” refers to a method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, intravenous administration, intra-arterial administration, intramuscular administration, intradermal administration, intrathecal administration, and subcutaneous administration. Administration can be continuous or intermittent. In some embodiments, administration is systemic rather than local.

The term “therapeutically effective amount” refers to an amount that is sufficient to effect beneficial or desirable biological or clinical results. That result can be reducing, alleviating, inhibiting, or preventing one or more symptoms of a condition, or any other desired alteration of a biological system. For example, in some embodiments, a therapeutically effective amount is an amount suitable to promote blood clot formation. In other embodiments, a therapeutically effective amount is an amount suitable to treat sepsis. Methods for determining a therapeutically effective amount are well known to those of skill in the art. A therapeutically effective amount will vary with several factors including, for example, the formulation of the composition used for therapy, the purpose of the therapy, and the subject being treated. A therapeutically effective amount of a composition may be delivered via single or multiple administrations. For example, a suitable daily dosage may be in the range of 3-20 mg/patient per day, 1-3 mg/patient per day, 20-100 mg/patient per day, or 20-50 mg/patient per day.

In some embodiments, the methods of the present invention are used to treat or prevent a condition that can be treated or prevented by enhancing or inhibiting APC's anticoagulant function. For example, in some embodiments, the methods are used to treat a condition in which blood clotting is desirable by inhibiting APC's anticoagulant function. Suitable conditions in which blood clotting is desirable include, without limitation, a hemorrhage (e.g., an intracranial hemorrhage, diffuse alveolar hemorrhage, intracerebral hemorrhage), a contusion (e.g., a brain contusion), a burn, gastrointestinal bleeding, uncontrolled bleeding, bleeding due to a transplantation (e.g., a stem cell transplantation, liver transplantation) or resection procedure, bleeding due to a surgery (e.g., a cardiac, spinal, orthopedic, neuro, oncological, or post-partum surgery), variceal bleeding, thrombocytopenia, idiopathic thrombocytopenic purpura, hemophilia, aortic aneurysm, reversal of an anticoagulant or antithrombotic (e.g., warfarin, heparin), bleeding due to a traumatic injury (e.g., a penetrating or blunt traumatic injury), menorrhagia, bleeding in cirrhosis (e.g., active variceal or non-variceal), deficiency of a clotting factor (e.g., factor VII), Glanzmann's thrombasthenia (e.g., refractory to platelet transfusion), and Bernard-Soulier syndrome. In some embodiments, the condition is an acute bleeding disorder. In some embodiments, the condition is an inherited bleeding disorder.

In other embodiments, the methods of the present invention are used to treat or prevent a condition that can be treated or prevented by enhancing or inhibiting one or more of APC's cytoprotective functions. For example, in some embodiments, the methods may be used to treat or prevent sepsis (Biochem Soc Trans 43:691-5, 2015), inflammation in acute ischemic disease (e.g., via providing neuroprotection in ischemic stroke (Ann Neurol 85:125-136, 2019) or providing cardioprotection in ischemic heart disease or heart failure (Int J Mol Sci 20:1762-1774, 2019)), coronavirus disease 2019 (COVID-19), diabetes (e.g., type 1 diabetes (J Biol Chem 287: 16356-16364, 2012), diabetic nephropathy (Proc Natl Acad Sci USA 110: 648-653, 2013; Nature Med 13: 1349-1358, 2007; J Thromb Haemost 10: 337-346, 2012; Blood 119: 874-883, 2012), diabetic ulcers, wounds (Am J Pathol 179: 2233-2242, 2011; Wound Repair Regen 13: 284-294, 2005; Circ Res 95: 34-41, 2004; J Invest Dermatol 125: 1279-1285, 2005; J Biol Chem 286: 6742-6750, 2011; Clin Haemorheol Microcirc 34: 153-161, 2006; Arch Dermatol 144: 1479-1483, 2008; Intern J Low Extrem Wounds 10: 146-151, 2011), amyotrophic lateral sclerosis (ALS) caused by a mutation that is SOD1 (J Clin Invest 119: 3437-3449, 2009), and multiple sclerosis (Nature 451: 1076-1081, 2008; J Immunol 191: 3764-3777, 2013). In some embodiments, the methods may be used to treat or prevent central nervous system injury (e.g., spinal cord ischemia), ischemic stroke, Alzheimer's disease, acute kidney injury, a lung disorder (e.g., acute lung injury, acute respiratory distress syndrome), or acute pancreatitis (Zhao et al., Int. J. Mol. Sci. 2019, 20, 903 at p. 12 of 20). In some embodiments, the methods are used to treat or prevent a condition that is associated with histones or NETs, such as a cancer (e.g., breast cancer, lung cancer, colorectal cancer, pancreatic cancer, blood cancer, neurological cancer, cutaneous cancer) or an inflammatory or autoimmune disease (e.g., psoriasis, rheumatoid arthritis, systemic lupus erythematosus).

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice, rats) for research purposes. In other embodiments, the methods are used to treat commercially important farm animals (e.g., cows, horses, pigs, rabbits, goats, sheep, chickens) or companion animals (e.g., cats, dogs). In a preferred embodiment, the subject is a human.

In an eighth aspect, the present invention provides uses of the antibodies disclosed herein as a medicament. The medicament may be for the treatment or prevention of (1) a condition that can be treated or prevented by inhibiting APC's anticoagulant function, and/or (2) a condition that can be treated or prevented by enhancing one or more of the pleiotropic cytoprotective functions of APC. Examples of such conditions are provided above.

The results of the functional assays provided in the examples can be used to determine if a particular anti-APC antibody described herein is useful for a particular indication. For example, antibodies that inhibit APC's anticoagulation activity while maintaining its cytoprotective activities are useful for treating disorders that require blood coagulation (e.g., acute bleeding). The importance of maintaining APC's cytoprotective activities for such indications is evidenced by Magisetty et al. (Blood (2022) blood.2021013119), discussed supra, in which the ability of two anti-APC antibodies to treat hemophilic arthropathy was assessed in a murine model. One of these antibodies, i.e., MAPC1591, inhibits APC's anticoagulant activity without inhibiting its cytoprotective activities. The second antibody, i.e., MPC1609, inhibits both the anticoagulant and cytoprotective activities of APC. The results of this study suggest that preserving APC's cytoprotective activities is useful for treating hemophilic arthropathy. Although no significant differences were observed between the ability of the two antibodies to inhibit APC's anticoagulant activity, administration of MAPC1591, and not MPC1609, markedly reduced the severity of hemophilic arthropathy. Antibodies that enhance APC's cytoprotective activities may be useful for treating disorders such as sepsis and inflammation in acute ischemic disease. Table 7 provides examples of some potential uses for specific antibodies based on the results of functional assays.

### EXAMPLES

Activated protein C (APC) is a pleiotropic coagulation protease with anticoagulant, anti-inflammatory, and cytoprotective activities. Selective modulation of these APC activities contributes to our understanding of the regulation of these physiological mechanisms and permits the development of therapeutics for the pathologies associated with these pathways. As is described in the following Example, a llama-derived nanobody library was generated targeting a non-active site of APC. From the library, 21 nanobodies were identified that selectively recognize APC compared to the protein C (PC) zymogen. Overall, three clusters of nanobodies were identified based on the competition for APC in bio-layer interferometry studies. APC functional assays (i.e., anticoagulant activity, histone H3 cleavage, and protease-activated receptor 1 (PAR1) cleavage) were used to understand the diversity of the antibodies. These functional assays revealed 13 novel nanobody-induced APC activity profiles via the selective modulation of APC pleiotropic activities, with potential for regulating specific mechanisms for therapeutic purposes. Within these, three nanobodies (LP2, LP8, LP17) inhibited all three APC functions. Four nanobodies (LP1, LP5, LP16, LP20) inhibited only two of the three APC functions. Mono-function inhibition specific to APC anticoagulation activity was observed with only two nanobodies (LP9, LP11). LP11 was also found to shift the ratio of APC cleavage of PAR1 at R46 relative to R41, which results in APC-mediated biased PAR1 signaling and APC cytoprotective effects. Thus, LP11 has an activity profile that is believed to promote hemostasis and cytoprotection in bleedings associated with hemophilia or coagulopathy by selectively modulating APC anticoagulation and PAR1 cleavage profile.

### Introduction

APC is a serine protease that has physiological functions in anticoagulation and cytoprotection (1, 2). APC circulates at a very low concentration (40 μM) in human blood, whereas its zymogen, protein C (PC), circulates at a much higher concentration (65 nM) (3). Thrombin/thrombomodulin activates PC via proteolytic cleavage at Arg169 and the release of a 12-amino acid peptide. APC down-regulates coagulation by inactivating factor Va (FVa) and factor VIIIa (FVIIIa) to terminate thrombin generation once hemostasis is achieved. This role in anticoagulation makes APC a potential therapeutic target for bleeding. Hemophilia patients with the APC resistant FV Leiden variant were observed to have reduced bleeding (4-6). Mice with acute traumatic coagulopathy experienced significantly less bleeding when treated with an APC-resistant ^{super}FVa variant (7). In addition, inhibitory antibodies specific for APC's anticoagulant activity are efficacious in hemophilic monkeys (8) and mice (9, 10). Furthermore, an engineered KRK-α₁-antitrypsin specific for inhibiting APC has also shown efficacy in reducing annualized bleed rates in hemophilia trials (11, 12). These findings demonstrate that APC inhibition promotes a pro-coagulation state.

APC also induces regenerative effects, including neurogenesis (13, 14) and wound healing (15). In addition, APC has protective effects against extracellular histones (16) released from immune or necrotic cells, which can lead to systemic inflammation, organ failure, and even mortality at pathological amounts (17). Protease activated receptor 1 (PAR1) is a major mediator of APC's cytoprotective activities. PAR1 has 2 cleavage sites for thrombin and APC. The cleavage at R41 by thrombin induces a pro-inflammatory response. In contrast, the cleavage at R46 by APC elicits biased PAR1 signaling and the activation of cytoprotective pathways (18, 19). Oligonucleotide-based aptamers, such as APC-167 (20), HS02 (21, 22), and G-NB3 (23), have been developed to target APC non-active sites to inhibit anticoagulant activity without interfering with cytoprotective functions. Recombinant APC protein (drotrecogin alfa (activated)) was developed as a treatment for severe sepsis (24). However, its initial efficacy was not replicated in a subsequent trial (25). With further understanding of the mechanism-of-action, it has been proposed that, with optimized dosing regimens for maximizing cytoprotective signaling, APC could provide benefits in a septic situation (2). More recently, 3K3A-APC (K191A/K192A/K193A), which has less than 10% APC anticoagulant activity while retaining cytoprotective activity, has advanced to clinical trials for ischemic stroke (26, 27). These APC cytoprotective roles are important, as the complete blockade of the APC active site by antibodies leads to many adverse effects including death (8, 28, 29).

The heavy chain IgG (hcIgG) is one type of camelid antibody that is composed of two heavy chains, each with one single variable domain on a heavy chain (V_{H}H) and two constant domains, CH2 and CH3 (30, 31). Antibodies with only the V_{H}H region (also known as nanobodies) represent the smallest antibody format.

To selectively target the different functions of APC, a nanobody phage library was constructed using peripheral blood mononuclear cells (PBMCs) of llamas that had been immunized with active site-blocked APC (APC-PPACK). From this library, a set of 21 llama nanobodies specific to APC non-active sites was identified. These 21 nanobodies have potential for selectively regulating the pleiotropic activity of APC for therapeutic use.

### Materials and Methods:

### Materials

Human APC, human APC-PPACK (Phe-Pro-Arg-chloromethylketone), human protein C, and factor Xa-PPACK were obtained from Haematologic Technologies (Essex Junction, VT); oligo(dT) primers from Invitrogen (Waltham, MA); V5 tag monoclonal antibody and HRP-anti-M13 from ThermoFisher (Waltham, MA); Maxisorp ELISA plate from Nunc (Rochester, NY); MAB230P monoclonal antibody against His-tag from Maine Biotechnology (Portland, ME); naïve llamas from Capralogics (Hardwick, MA); Protac® from Pentapharm (Parsippany, NJ); STA-PTT Automate 5, STA Neoplastin CI Plus 5 from Stago (Parsipanny, NJ); normal human plasma from George King Bio-Medical (Overland Park, KS); PC-depleted plasma from Affinity Biologicals (Anacaster, ON, Canada); TPP-4885 Fab anti-APC in IgG1/κ framework (in-house); plasma-derived human APC and human PC from Enzyme Research Laboratories (South Bend, IN, USA); Spectrozyme PCa from American Diagnostica (Pfungstadt, Germany); calf thymus histone H3 from Sigma-Aldrich (St. Louis MO); Odyssey marker from LI-COR (Lincoln, NE); and One-Step Blue Protein Stain from Biotium (Fremont, CA).

### Generation of Llama Nanobodies Against the Non-Active Site of Human APC

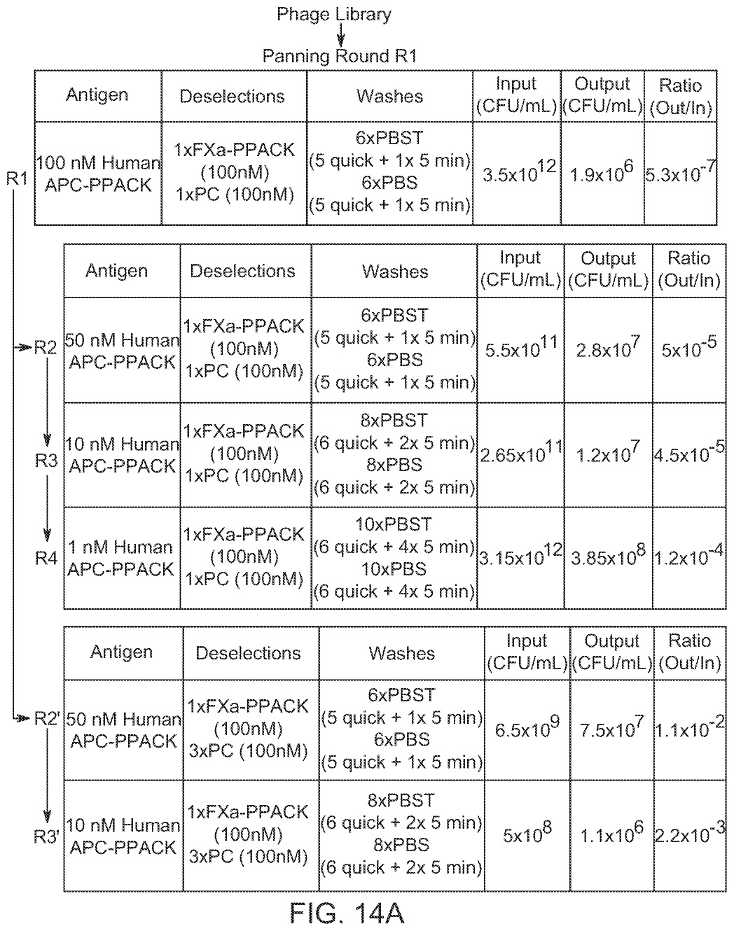

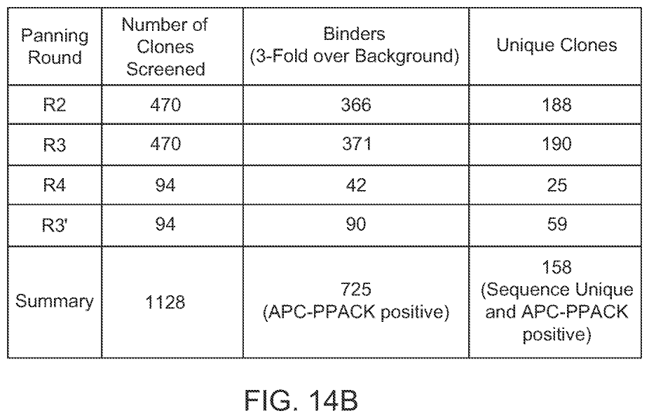

Two male llamas were immunized with APC-PPACK on day 1 and day 77. Peripheral blood mononuclear cells (PBMCs) were isolated 5 days after the last immunization. Total RNA was extracted using the RNeasy kit (Qiagen). cDNA was synthesized from RNA using oligo(dT) primers and SuperScript III First-Strand Synthesis for RT-PCR (ThermoFisher). A nanobody phage display library was constructed by directly amplifying the nanobody using framework region 1 (FR1) and hinge region specific primers and cloning the amplicon into a vector with a V5 and His-tag. APC-PPACK binders were enriched by panning against APC-PPACK and negatively deselected with factor Xa-PPACK and PC (FIG. 14).

### Phage and Bacterial Periplasmic Extracts (PPEs) ELISA Screening

Plates were coated overnight at 4° C. with 2 μg/mL APC-PPACK, PC, or FXa-PPACK, blocked with 2% BSA at room temperature (RT) for 1 hour, and used to screen clones from panning rounds R2, R3, and R3′ (FIG. 14). Phage were blocked in 4% BSA at RT for 1 hour and were detected with 1:5000 HRP-anti-M13 mAb. Plates were developed with tetramethylbenzidine (TMB) and quenched with 2N H₂SO₄. Clones that exhibited an OD₄₅₀ that was at least three-fold higher in antigen-coated wells as compared to in blocked wells were selected for sequencing. PPEs generated from individual clones in panning round R4 were screened similarly by performing enzyme-linked immunosorbent assays (ELISA) using mouse anti-V5 monoclonal antibody (1:5000) as the primary antibody and goat HRP-anti-mouse IgG (1:5000) as the secondary antibody.

### Expression and Purification of Nanobodies

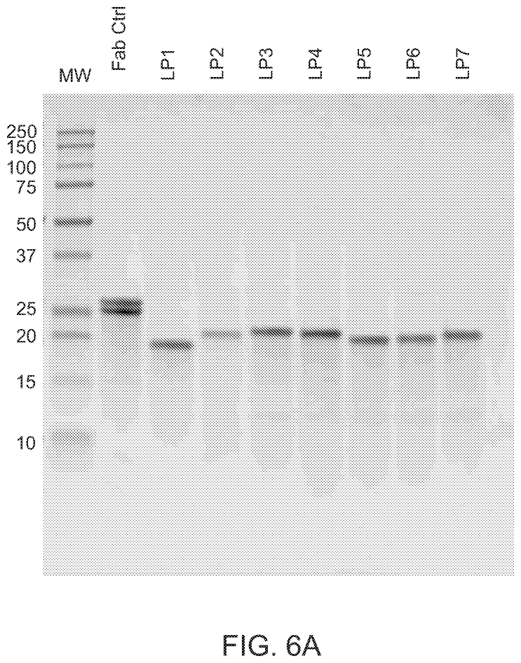

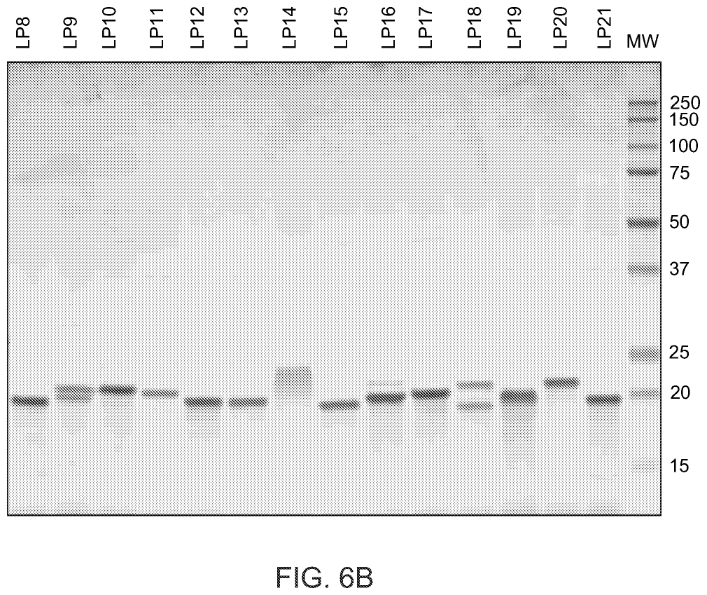

E. coli. grown in 2YTGC supplemented with 0.1% glucose were transformed with plasmids encoding nanobodies fused to a 6×His-tag. Nanobody expression was induced using 1 mM IPTG at an OD₆₀₀ of about 0.5 (25° C.). Nanobodies in the periplasm were purified with 1 mL of immobilized metal chelate affinity chromatography (IMAC) resin at 4° C. The resin was washed with 3 times with 1 mL 10 mM imidazole/PBS and bound antibodies were eluted with 3 mL 100 mM imidazole/PBS. The buffer was exchanged into PBS containing 1 mM CaCl₂. The purity of nanobodies was characterized by SDS-PAGE (FIG. 6). LP11 was also expressed in mammalian CHO cells and purified by IMAC as described above.

### Epitope Binning on Nanobodies

Binning experiments were performed on an Octet® HTX at 25° C. Anti-V5 mouse antibody (5 μg/mL) was loaded onto anti-mouse IgG Fc capture Octet® HTX sensors. The loaded sensors were dipped into nanobody solutions (5 μg/mL) to capture V5-tagged nanobodies as ligand. The sensors were dipped into 500 nM APC for 80 seconds, followed by dissociation for 30 seconds, and in 200 nM APC non-active site specific TPP-4885 Fab for 80 seconds. The binding of APC to nanobody was detected by measuring the reflected light spectral shift on the sensor surface upon nanobody-APC interaction at 25° C. The binding of TPP-4885 Fab to this nanobody-APC complex on the sensor was monitored similarly.

To further understand the epitope diversity of the nanobodies that do not compete with TPP-4885 Fab, a classical sandwich BLI epitope assay was performed with nanobodies that included a V5 tag at 25° C. The anti-mouse IgG Fc capture sensors loaded with anti-V5 antibody were dipped into nanobody solutions (10 μg/mL), followed by 500 nM APC for 80 seconds, dissociation for 20 seconds, and 200 nM nanobody analytes for 80 seconds. The normalized binning data were analyzed with the pvclust package (49). The y-axis of the clustergram, referred to as Height, is a measure of dissimilarity between nanobodies. Approximate unbiased (AU) p-values were computed using multi-scale bootstrap resampling to confirm bin assignments.

### Binding Kinetics Analysis by Bio-Layer Interferometry

An Octet® HTX instrument (Sartorius, Fremont, CA) sensor surface with anti-mouse IgG was loaded with anti-V5 mouse antibody (10 μg/mL). The loaded sensor was immersed in a nanobody solution (5 μg/mL in the first analysis, 10 μg/mL in the second analysis) to immobilize the V5-tagged nanobody. The sensor with the immobilized nanobody was immersed in APC dilutions (78.12-5000 nM in PBS, 1 mM CaCl₂, 0.1% w/v Tween20, with or without 0.5 mg/mL BSA) for 60 seconds. Following APC binding, the surface was immersed in assay buffer for 120 seconds to allow for dissociation. Bio-layer interferometry (BLI) measurements were collected during binding and dissociation phases (25° C.). The surface was regenerated between samples with 10 mM glycine buffer, pH 1.7. Kinetic constants (kₒₙ, k_{off}, and K_{D}) were calculated using a monovalent (1:1) binding model. Each nanobody was tested against 5 different concentrations of APC.

### Effect of Nanobodies on APC Anticoagulant Activity

The PC-activating, snake venom-derived reagent Protac® was used to generate APC in normal plasma, and the activated partial thromboplastin time (APTT) clotting time was measured to test the effects of the nanobodies on APC's anticoagulant activity. Normal plasma samples (50 μL) with anti-APC nanobodies were mixed with Protac® (25 μL; 1 U/mL) and Stago reagent STA-PTT (75 μL) at 37° C. After a 4-minute incubation, 75 μL of 25 mM CaCl₂) solution was added to initiate clotting. The Protac®-APTT clotting time of normal human plasma serially diluted with PC-depleted plasma was used as a standard curve. For FVIII inhibitor plasma, 0.5 μg/ml (16 BU/ml) of the FVIII inhibitory antibody GMA-8015 (Green Mountain) was added to normal plasma and incubated for 2 hours prior to the Protac®-APTT assay.

### Effect of Nanobodies on APC-Mediated Histone H3 Cleavage

APC (50 nM) and anti-APC nanobodies (500 nM) were preincubated in HBS buffer (20 mM Hepes, 147 mM NaCl, 3 mM KCl, pH 7.4) with 100 μg/mL BSA and 2 mM CaCl₂. After 30 minutes, the mixtures were added to 100 μg/mL histone H3. Over a period of 2 hours, the mixtures were subsampled at different time points and quenched with reducing sample buffer. The samples were heated for 15 min at 95° C., centrifuged for 5 min at 4000 rpm, vortexed briefly, and loaded into a 12% Bis-Tris gel (Bio-Rad) with MES running buffer (30 μl/well; 750 ng H3). The 20 kDa marker in the Odyssey standard (10 μL) was used as reference for normalization between gels. Gels were stained overnight with Biotum One-Step Blue Protein Stain (50 mL/gel). Protein bands were scanned on a Licor Odyssey (700 channel, intensity 6, 1 mm offset, resolution 169 μm) and analyzed with Image Studio V5.2.

### Effect of Nanobodies on APC-Mediated SEAP-PAR1 Cleavage

HEK293 cells expressing wild-type EPCR and a PAR1 cleavage reporter construct with an N-terminal secreted embryonic alkaline phosphatase (SEAP-PAR1) (18,38) were grown in 96-well plates until confluent. Cells were washed with Hanks' balanced salt solution supplemented with 1.3 mM CaCl₂, 0.6 mM MgCl₂, and 0.1% BSA (endotoxin free HMM2). APC (50 or 100 nM) and nanobodies were preincubated for 30 minutes in HMM2 before addition to the cells. After 60 minutes, SEAP release was determined using p-nitrophenyl phosphate. PAR1 cleavage was expressed as the percentage of the total SEAP activity present on the cells versus background. R41Q-SEAP-PAR1 and R46Q-SEAP-PAR1 HEK293 cell lines were used to evaluate the effect of the nanobodies on APC's selectivity for cleavage at R46 versus R41 (18).

### Endothelial Barrier Integrity Assay

The effects of nanobodies on APC-mediated endothelial barrier integrity were measured using a transendothelial electrical resistance (TEER) assay (iCelligence system, ACEA, San Diego, CA), as previously described (18, 39). EA.hy926 endothelial cells were grown at 2.5×10⁴ cells/500 μL per well to confluence in 8-well E-plate L8 (Agilent Technologies, Santa Clara, CA). DMEM (Invitrogen) containing 10% fetal calf serum was replaced with serum-free media containing 0.1% BSA 2 hours before the addition of APC (40 nM) and LP11. Thrombin (0.25 nM) was added 30 minutes later to induce endothelial permeability. The changes in electrical resistance of the confluent monolayers were measured as the normalized cell index in real time.

### In Vitro Plasma Half-Life Assay.

The effects of the nanobodies on the sensitivity of APC to physiological inhibitors were assessed using an in vitro plasma half-life assay. In this assay, APC (70 nM) and antibody (700 nM) are preincubated for 30 minutes and normal pooled plasma is added to 90% (v/v). Samples are collected at various timepoints (0-90 minutes) and quenched in ice-cold TBS. The chromogenic activity of APC is determined using Pefachrome® PCa. Background chromogenic activity of plasma (without APC addition) is subtracted from the measured signal, and APC activity is normalized to the t=0 timepoint to account for the effects of the antibody on APC's chromogenic activity. Half-life (T_{1/2}) is determined by one-phase exponential decay curve fitting.

### Results:

### Generation of Nanobodies Specific for the Non-Active Site of APC

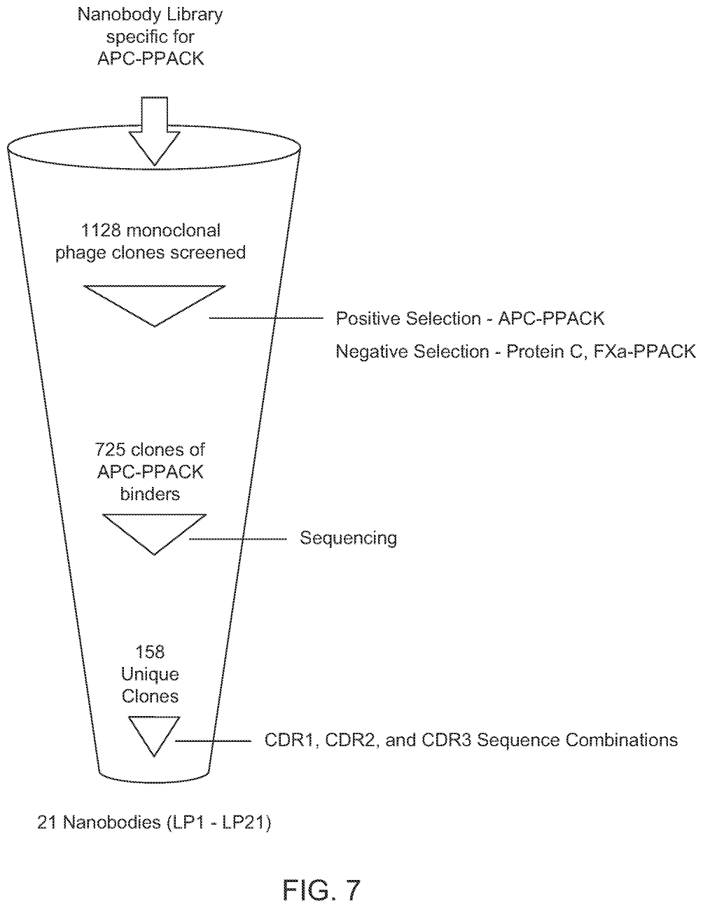

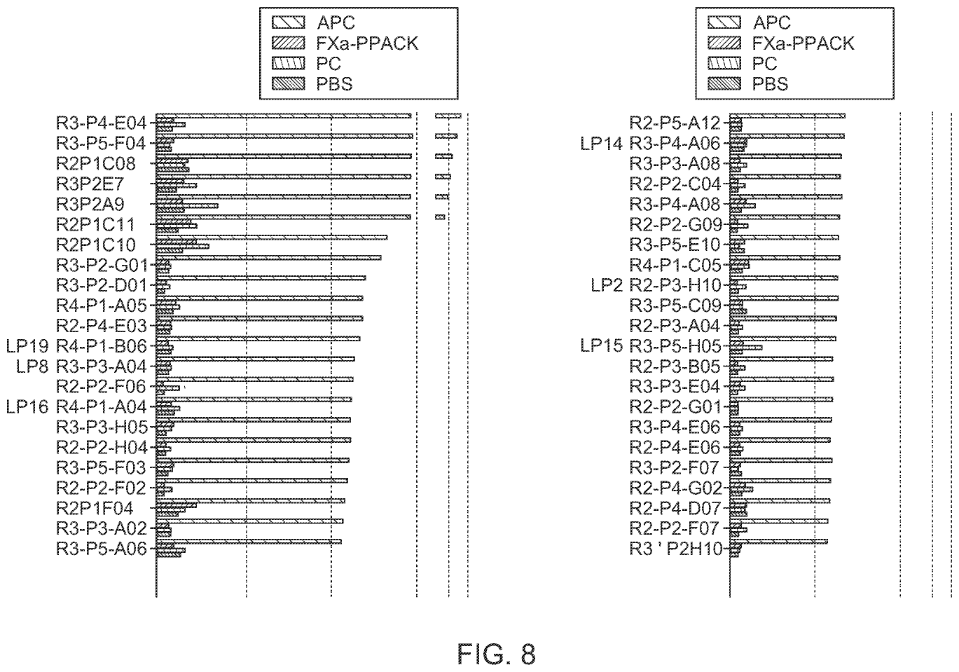

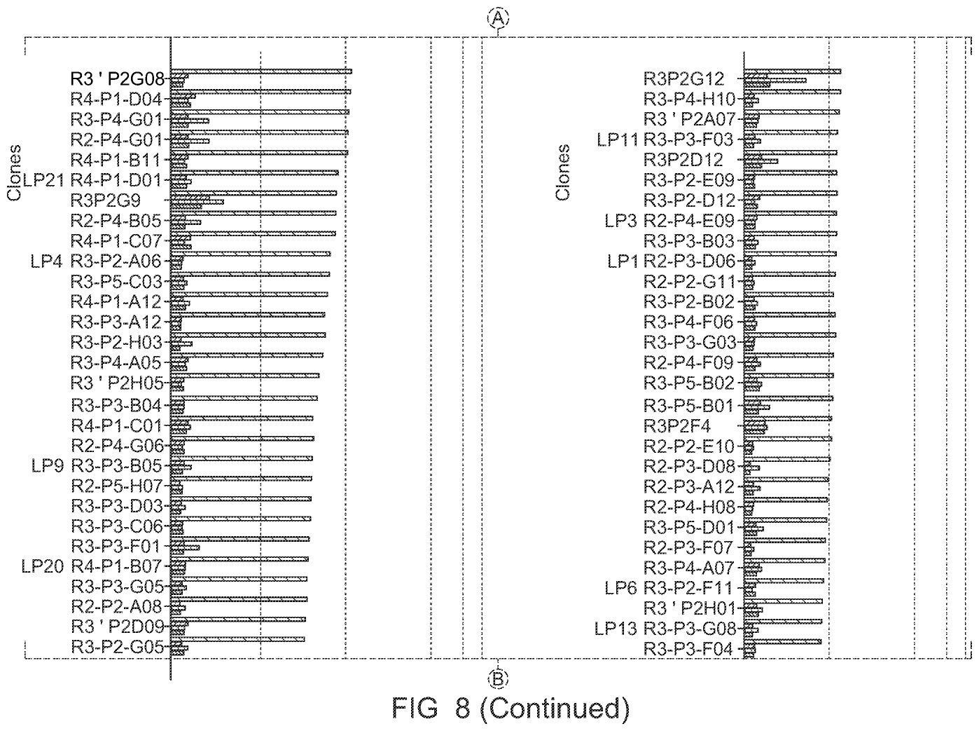

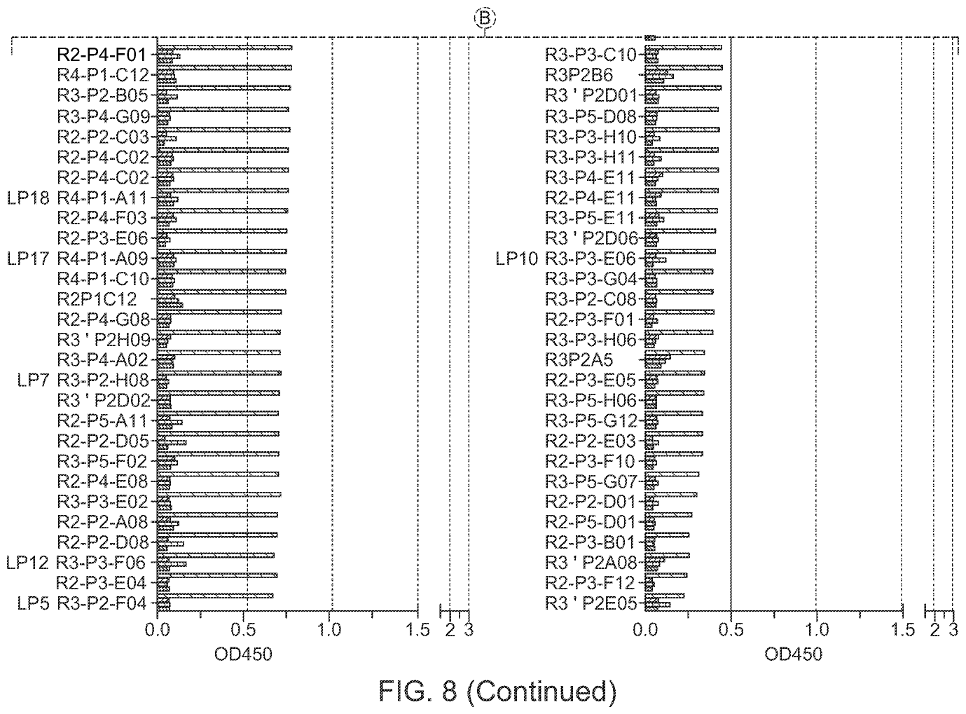

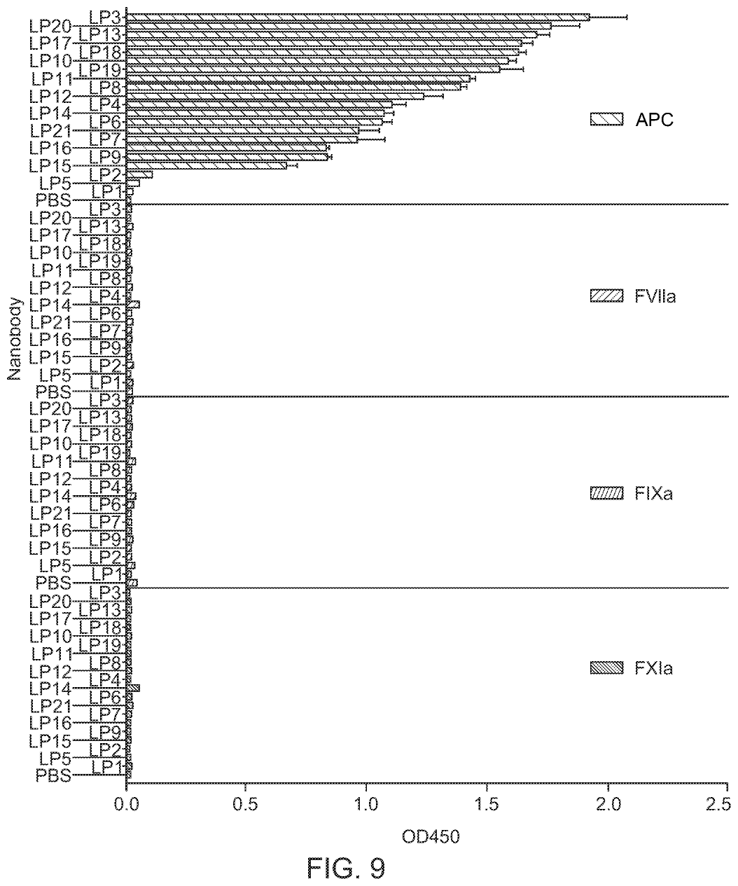

APC-PPACK was used as an immunogen for generating nanobodies against the non-active site of APC in llamas. After immunization with two doses of APC-PPACK, PBMCs were collected and nanobody binding regions were cloned to create a nanobody library. The library was panned against APC-PPACK and PC to select clones specific for APC but not PC. After panning round R1, panning was continued in two arms that differed in the stringency of negative selection for PC (FIG. 14). Both the first arm (R1, R2, R3, R4) and the second arm (R1, R2′, R3′) led to the enrichment of APC-specific clones. Of the total 1128 phage clones selected from R2, R3, R4, and R3′ (FIG. 7), 725 clones were identified as selective APC-PPACK binders that lacked non-specific binding to PC or FXa-PPACK (FIG. 8). After sequencing, 158 clones were found to be unique. Based on their CDR1, CDR2, and CDR3 sequences, these 158 clones were grouped into 18 families from which 21 nanobodies (named LP1 to LP21) with the highest binding within each family were selected. Additional non-specific binding studies against FVIIa, FIXa, and FXIa were performed and further demonstrated the selectivity of the LP1-LP21 nanobodies towards APC (FIG. 9).

### Binding Kinetics and Epitope Binning

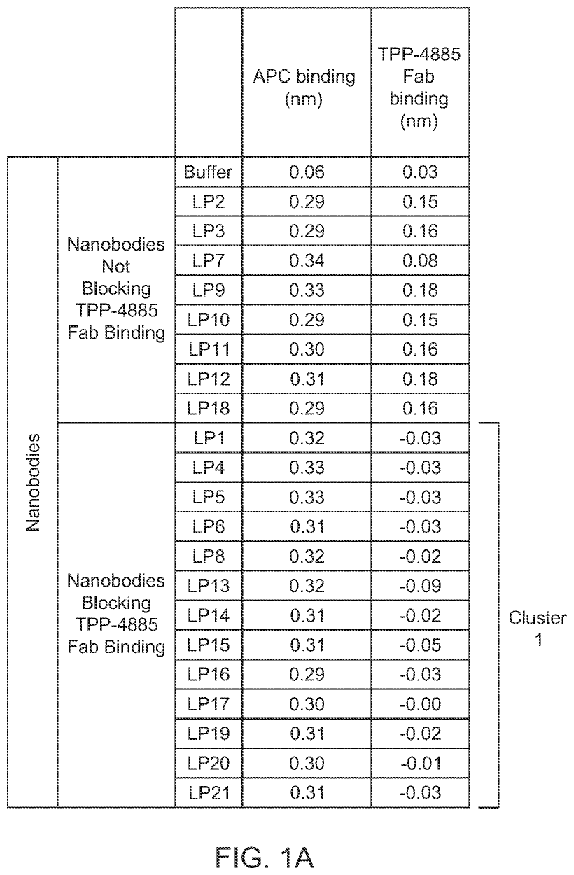

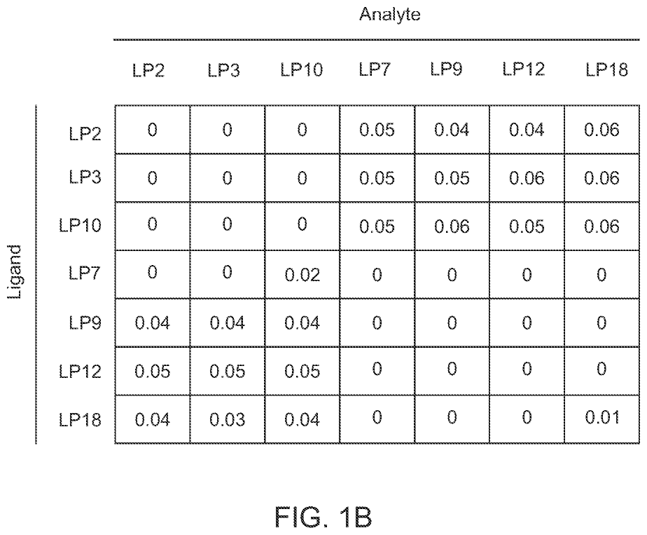

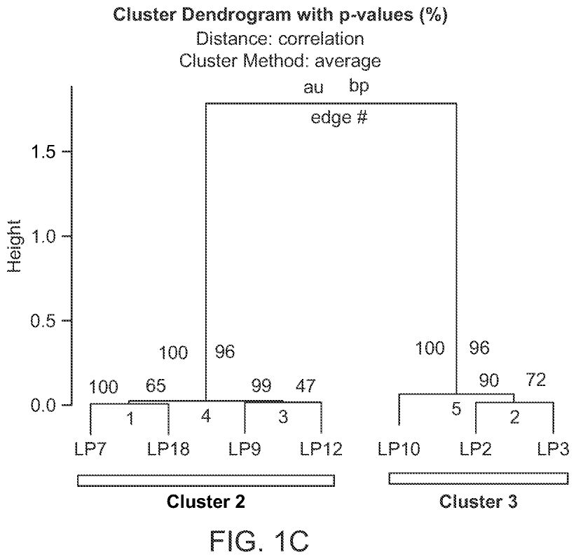

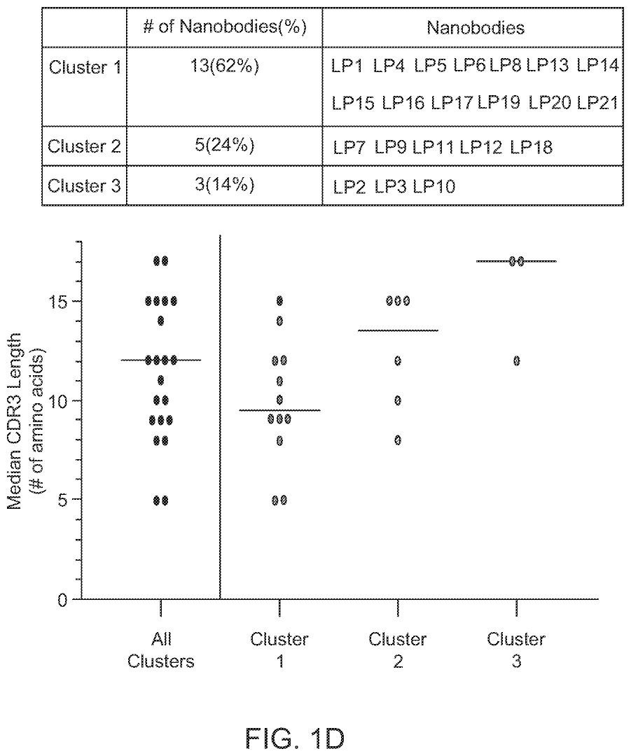

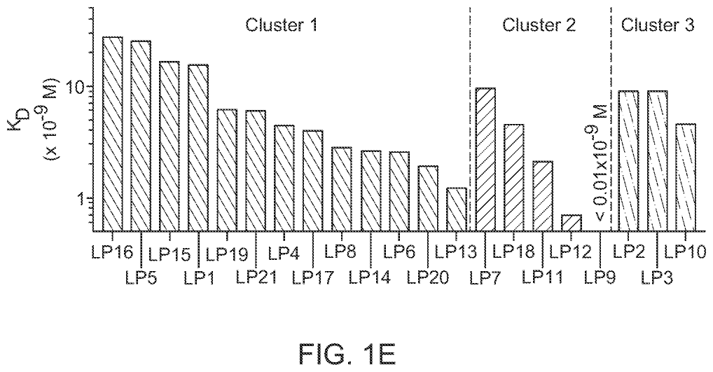

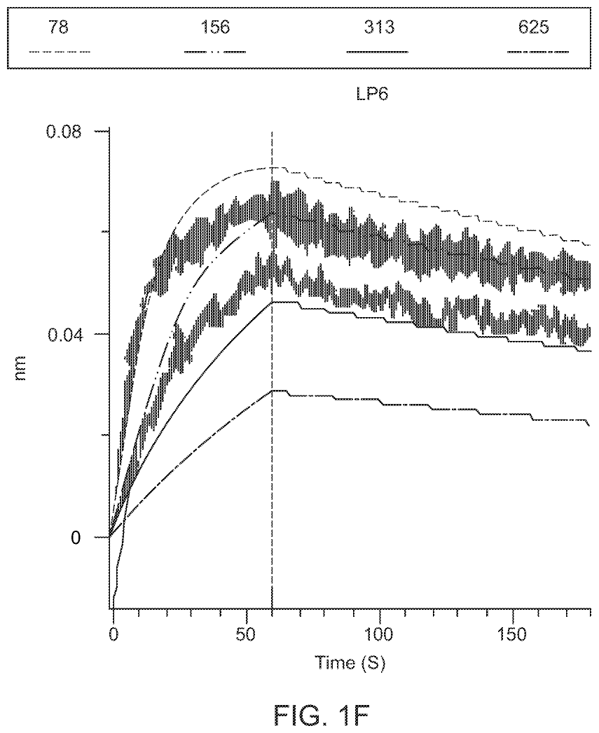

To determine if any of the nanobodies bind to the same epitope as the previously reported non-active site-specific anti-APC Fab antibody TPP-4885 (8), a BLI-based epitope binning study was performed. The nanobodies were tested pairwise with TPP-4885 Fab. Thirteen clones blocked TPP-4885 Fab binding and binned into Cluster 1 (FIG. 1A). Nanobodies that did not block TPP-4885 Fab binding to APC were selected for additional binning experiments by BLI except for LP11 because it has greater than 90% CDR sequence identity to LP9 (Table 4). Thus, LP9 was used as a surrogate for LP11. Based on the response measurements by BLI, LP7, LP9, LP12, and LP18 did not compete with LP2, LP3, and LP1 (FIG. DB). LP7 had a weak response measurement as a ligand but appeared to bind well as an analyte. This might be due to the faster k_{off} of LP7. Clustering analysis showed the clones could be binned into Cluster 2 and Cluster 3 FIG. 1C). Most nanobodies (62%) are in Cluster 1. The lower number of nanobodies for Cluster 2 (24%) and 3 (14%) implies that these epitope bins were less accessible and/or more complex for immune recognition and antibody development. These epitope bins also appear to be targetable only by nanobodies with longer CDR3. Unlike Cluster 1 nanobodies, which had a median CDR3 length of 9.5 amino acids, the CDR3 of Cluster 2 and 3 nanobodies had a median length of 13.5 and 17 amino acids, respectively (FIG. 11D, Table 4). These nanobodies had kₒₙ values ranging from 1.26×10⁵ to 6.22×10⁵ l/M·sec and k_{off} values ranging from 2.59×10⁻⁴ to 1.37×10⁻³ l/sec. The nanobody LP9 had a particularly low k_{off} value, estimated to be around 6.33×10⁻⁷ l/sec. Two nanobodies had sub-nanomolar K_{D} values, while 15 nanobodies had single digit nanomolar K_{D} values, and four nanobodies had K_{D} values ranging from 16.5 to 27.5 nM (FIG. 1E, Table 5).

TABLE 4
Amino acid sequences of CDR1, CDR2, and CDR3 for LP1-LP21.

| Nanobody | CDR1 | CDR2 | CDR3 |
|---|---|---|---|
| LP1 | GSTFPINV | VAFIGRSGTT | NLGDY |
| | (SEQ ID NO: 45) | (SEQ ID NO: 203) | (SEQ ID NO: 361) |
| LP2 | GRPISAYT | ITSSAGTT | AADDQGDNWYDH |
| | (SEQ ID NO: 54) | (SEQ ID NO: 212) | (SEQ ID NO: 370) |
| LP3 | GTIESIND | ITSGGST | NAEVKRHYYYDSAGYRY |
| | (SEQ ID NO: 61) | (SEQ ID NO: 219) | (SEQ ID NO: 377) |
| LP4 | GSTFNFRI | INSGGSA | HAKKVEPTLFTTTY |
| | (SEQ ID NO: 74) | (SEQ ID NO: 232) | (SEQ ID NO: 390) |
| LP5 | GSIFTTAT | ITTGGST | NAATRLSSTNY |
| | (SEQ ID NO: 82) | (SEQ ID NO: 240) | (SEQ ID NO: 398) |
| LP6 | GSIGRLNV | ISSDNKK | NVRWTMGGNY |
| | (SEQ ID NO: 84) | (SEQ ID NO: 242) | (SEQ ID NO: 400) |
| LP7 | GTTFDMNS | ISMGGDT | NAERIWPNRRTF |
| | (SEQ ID NO: 88) | (SEQ ID NO: 246) | (SEQ ID NO: 404) |
| LP8 | GRTFSSGA | IRWSDERT | RSTVGNLLY |
| | (SEQ ID NO: 90) | (SEQ ID NO: 248) | (SEQ ID NO: 406) |
| LP9 | GRTFVGSYV | SSRSTGNT | ATRASINYNAATWPY |
| | (SEQ ID NO: 95) | (SEQ ID NO: 253) | (SEQ ID NO: 411) |
| LP10 | GTIESIND | VSSSGMT | KAHISRSYYYDSLGYDY |
| | (SEQ ID NO: 101) | (SEQ ID NO: 259) | (SEQ ID NO: 417) |
| LP11 | GRTFVGSYV | SSRSTGNT | ATRASVNYNYAAWPY |
| | (SEQ ID NO: 103) | (SEQ ID NO: 261) | (SEQ ID NO: 419) |
| LP12 | GLTASTYA | VDWKGDT | AARGSPGYVARLYAH |
| | (SEQ ID NO: 105) | (SEQ ID NO: 263) | (SEQ ID NO: 421) |
| LP13 | GTTFTIED | SRGGGNA | IAKTKATTK |
| | (SEQ ID NO: 109) | (SEQ ID NO: 267) | (SEQ ID NO: 425) |
| LP14 | GSTSSIVI | INNGGDV | SARTPSLRVTWF |
| | (SEQ ID NO: 116) | (SEQ ID NO: 274) | (SEQ ID NO: 432) |
| LP15 | GTTFSFVY | ISSDGST | FWNGHTNK |
| | (SEQ ID NO: 142) | (SEQ ID NO: 300) | (SEQ ID NO: 458) |
| LP16 | GTTFSANT | ISSSGTT | NAILPSRSI |
| | (SEQ ID NO: 144) | (SEQ ID NO: 302) | (SEQ ID NO: 460) |
| LP17 | GTTSSLNL | MTSDGRT | YANPFLGSWSNY |
| | (SEQ ID NO: 146) | (SEQ ID NO: 304) | (SEQ ID NO: 462) |
| LP18 | GFTFSTKW | IDAGGTST | ARSEGGLF |
| | (SEQ ID NO: 147) | (SEQ ID NO: 305) | (SEQ ID NO: 463) |
| LP19 | GSTFSINV | IGRSGTP | HLGDT |
| | (SEQ ID NO: 149) | (SEQ ID NO: 307) | (SEQ ID NO: 465) |
| LP20 | GSTVRVTA | IDNGGST | FVIAIPDSAPRPRAY |
| | (SEQ ID NO: 150) | (SEQ ID NO: 308) | (SEQ ID NO: 466) |
| LP21 | GSTFGRNV | ITTSGST | WTAIAGQGEY |
| | (SEQ ID NO: 157) | (SEQ ID NO: 315) | (SEQ ID NO: 473) |

TABLE 5
The kₒₙ, k_{off}, and K_{D} of the 21 nanobodies. Each nanobody was
tested against 5 different concentrations of APC. R² of these
binding kinetic measurements ranged from 0.92 to 0.99.

| | kₒₙ | k_{off} | K_{D} |
|---|---|---|---|
| Nanobody | (×10³ 1/Ms) | (×10⁻³ 1/s) | (×10⁻⁹ M) |
| LP1 | 590 | 9.0 | 15 |
| LP2 | 130 | 1.1 | 8.8 |
| LP3 | 180 | 1.6 | 8.8 |
| LP4 | 550 | 2.7 | 4.4 |
| LP5 | 450 | 11 | 25 |
| LP6 | 310 | 0.78 | 2.5 |
| LP7 | 440 | 4.1 | 9.4 |
| LP8 | 620 | 1.7 | 2.8 |
| LP9 | 210 | 0.00063 | 0.0030 |
| LP10 | 230 | 1.1 | 4.5 |
| LP11 | 330 | 0.69 | 2.1 |
| LP12 | 380 | 0.26 | 0.68 |
| LP13 | 350 | 0.41 | 1.2 |
| LP14 | 420 | 1.1 | 2.6 |
| LP15 | 400 | 6.6 | 16 |
| LP16 | 500 | 14 | 27 |
| LP17 | 320 | 1.2 | 3.9 |
| LP18 | 240 | 1.1 | 4.5 |
| LP19 | 410 | 2.5 | 6.1 |
| LP20 | 380 | 0.70 | 1.9 |
| LP21 | 600 | 3.5 | 5.9 |

### Effect on APC-Mediated Cleavage of S-2366

Eight nanobodies had no effect on APC-mediated cleavage of the chromogenic substrate S-2366 (FIG. 10). Five nanobodies induced partial inhibition, and 8 nanobodies showed an enhancement of S-2366 cleavage. Among the inhibitory nanobodies, two were non-competitive inhibitors, and three were competitive inhibitors for 5-2366 cleavage (FIG. 11, Table 6). Of the 8 S-2366 cleavage-enhancing nanobodies, 4 enhanced cleavage by increasing Vₘₐₓ, and the other 4 enhanced cleavage by decreasing K_{M}. The lack of complete inhibition of S-2366 cleavage demonstrated that these 21 anti-APC nanobodies target a non-active site.

TABLE 6
Summary of the effects of the nanobodies on the
Vₘₐₓ and Kₘ of APC-mediated S-2366 cleavage.

| | | Effect on APC | | | |
|---|---|---|---|---|---|
| | | in S-2366 | | | |
| | Nanobody | Hydrolysis | Mode | | |
| Cluster | LP1 | No effect | — | — | — |
| 1 | LP4 | Enhancement | Enhancement (Type 2) | Same Vₘₐₓ | ↓ Kₘ |
| | LP5 | Inhibition | Non-competitive Inhibition | ↓ Vₘₐₓ | Same Kₘ |
| | LP6 | Inhibition | Competitive Inhibition | Same Vₘₐₓ | ↑ Kₘ |
| | LP8 | Enhancement | Enhancement (Type 2) | Same Vₘₐₓ | ↓ Kₘ |
| | LP13 | Inhibition | Competitive Inhibition | Same Vₘₐₓ | ↑ Kₘ |
| | LP14 | Enhancement | Enhancement (Type 2) | Same Vₘₐₓ | ↓ Kₘ |
| | LP15 | No effect | — | — | — |
| | LP16 | No effect | — | — | — |
| | LP17 | No effect | — | — | — |
| | LP19 | No effect | — | — | — |
| | LP20 | Enhancement | Enhancement (Type 2) | Same Vₘₐₓ | ↓ Kₘ |
| | LP21 | Inhibition | Competitive Inhibition | Same Vₘₐₓ | ↑ Kₘ |
| Cluster | LP7 | No effect | — | — | — |
| 2 | LP9 | Enhancement | Enhancement (Type 1) | ↑ Vₘₐₓ | Same Kₘ |
| | LP11 | Enhancement | Enhancement (Type 1) | ↑ Vₘₐₓ | Same Kₘ |
| | LP12 | Inhibition | Non-competitive Inhibition | ↓ Vₘₐₓ | Same Kₘ |
| | LP18 | Enhancement | Enhancement (Type 1) | ↑ Vₘₐₓ | Same Kₘ |
| Cluster | LP2 | No effect | — | — | — |
| 3 | LP3 | Enhancement | Enhancement (Type 1) | ↑ Vₘₐₓ | Same Kₘ |
| | LP10 | No effect | — | — | — |

### Effect on Clotting Time in Plasma

The effect of the nanobodies on APC anticoagulant activity was screened using a Protac®-modified APTT assay. Protac® (a single chain glycoprotein derived from snake venom that activates protein C) was added to plasma to generate APC. Protac® prolonged the APTT clotting time from an average of 40.7 s to 262.0 s, depending on the concentration of PC in the plasma (FIG. 2A). The nanobodies were screened twice at 300 nM and showed consistent potency rank order between screens (FIG. 2B). All nanobodies reversed the prolonged clot time at least partially. LP2, LP8, LP9, LP11, LP17, and LP20 inhibited APC-induced clotting by >90%, with the prolonged clotting time reversed to below 80 s. LP20 was the most potent inhibitor and almost fully corrected clotting times (FIG. 2C). These data demonstrate that there are at least three different epitopes (Cluster 1, 2, and 3) on APC that can be bound by nanobodies to inhibit this protein's anticoagulant activity.

### Effect on APC-Mediated Histone H3 Cleavage

APC inhibits histone-induced cytotoxicity via proteolytic cleavage (16). In this assay, APC was incubated with histone H3 in the presence of nanobodies and the cleavage of histone H3 was monitored by SDS-PAGE (FIG. 3A). Nine nanobodies had minimal impact on APC-mediated histone H3 cleavage (FIG. 3B), one (LP14) enhanced H3 cleavage (FIG. 3C), and twelve nanobodies inhibited APC-mediated H3 cleavage resulting in <75% residual activity (FIG. 3D) with LP21 being the most potent (FIG. 3E).

### Effect on APC-Mediated SEAP-PAR1 Cleavage

Nanobodies that inhibited PAR1 cleavage by APC could be found in all 3 clusters, with LP1, LP8, LP17, LP19, and LP21 showing the most inhibition. In contrast, the nanobodies LP3, LP9, LP11, and LP18 showed minimal inhibition even at their highest concentration of 500 nM (FIG. 4A). At a lower concentration range, 0.7-6 nM, these four nanobodies reduced PAR1 cleavage down to 73-82% of controls. These nanobodies belonged to Cluster 2 (LP9, LP11, LP18) and Cluster 3 (LP3), respectively. All nanobodies in Cluster 1 inhibited PAR1 cleavage. APC can cleave PAR1 at both R41 and R46, with the latter cleavage resulting in cytoprotective signaling. Therefore, two PAR1 cleavage-inhibitory nanobodies and two PAR-1 cleavage-sparing nanobodies were evaluated for their effects on APC cleavage pattern using R41Q-SEAP-PAR1 and R46Q-SEAP-PAR1 cell lines (FIG. 4B). In the presence of inhibitory nanobodies LP8 and LP20, the cleavages at both sites by APC (at 50 nM) were inhibited in a dose-dependent manner. At high concentrations of LP8 and LP20, the cleavage ratio shifted to below 1, a less cytoprotective PAR1 cleavage profile (FIG. 4C). In comparison, LP11 and LP18 appeared to induce a curvilinear cleavage response for both R46 and R41. At 0.7 to 6 nM, the cleavage of R41 was suppressed to a larger extent than R46. At 6 nM and higher, the suppression of cleavage was reversed. Interestingly, the R46/R41 cleavage ratio was above 1 for the entire concentration range of the assay, suggesting an enhanced cytoprotection effect by LP11 and LP18 (FIG. 4D).

### Effect on the Plasma Half-Life of APC

An in vitro plasma half-life assay was used to determine the effects of the 21 nanobodies on the sensitivity of APC to physiological inhibitors. This assay identified V_{H}H antibodies that increase, decrease, and have no effect on the plasma half-life of APC (Table 7). An increase in half-life indicates that the binding of these antibodies reduces the susceptibility of APC to inhibition by physiological inhibitors present in plasma.

TABLE 7
Results of an in vitro plasma half-life assay showing the effects
of the 21 nanobodies on the mean in vitro half-life (T_{1/2}) of
APC in plasma. The standard deviations (SD) and P values were
generated via comparison to the no antibody condition.

| Nanobody | T_{1/2} (min; mean) | SD | P value |
|---|---|---|---|
| — | 29.2 | 3.5 | ref |
| — | 29.4 | 3.0 | ref |
| LP1 | No decay in APC | — | <0.0001 |
| | activity | | |
| LP2 | 38.2 | 3.8 | ns |
| LP3 | 28.8 | 1.2 | ns |
| LP4 | 33.0 | 2.0 | ns |
| LP5 | 283.8 | 101.3 | <0.0001 |
| LP6 | 93.1 | 17.1 | 0.04 |
| LP7 | 33.9 | 3.3 | ns |
| LP8 | No decay in APC | — | |
| | activity | | |
| LP9 | 27.7 | 2.0 | ns |
| LP10 | 29.3 | 2.4 | ns |
| LP11 | 25.0 | 0.8 | ns |
| LP12 | 50.1 | 7.5 | ns |
| LP13 | 67.1 | 15.8 | ns |
| LP14 | 14.8 | 2.0 | ns |
| LP15 | 36.4 | 4.7 | ns |
| LP16 | 65.1 | 9.7 | ns |
| LP17 | 102.2 | 17.0 | 0.01 |
| LP18 | 16.1 | 2.0 | ns |
| LP19 | No decay in APC | — | <0.0001 |
| | activity | | |
| LP20 | 85.6 | 12.6 | ns |
| LP21 | No decay in APC | — | <0.0001 |
| | activity | | |

### Activity Profiles

The 21 nanobodies were categorized into 15 different activity profiles based on their clusters and effects in 3 APC functional assays (Table 8). Three nanobodies (LP2, LP8, LP17) strongly inhibited all 3 APC functional activities. Several nanobodies were found to inhibit only 2 of the 3 functions: LP16 and LP20 inhibit anticoagulation and PAR1 cleavage, and LP1 and LP5 inhibit H3 and PAR1 cleavage. Because these nanobodies spare only 1 of 3 APC functions, they could be useful for dissecting the pleiotropic effects of APC, especially in an in vivo setting. No nanobody was identified for strongly inhibiting only PAR1 cleavage or histone H3 cleavage. Strong mono-function inhibition of APC anticoagulant activity was observed only for LP9 and LP11.

TABLE 8
Classification of the 21 nanoantibodies into 15 antibody profile categories
based on their ability to affect various activities of APC.

| | | | | SEAP- | Sensitivity to | | |
|---|---|---|---|---|---|---|---|
| | | Anticoagulation | Histone | PAR1 | Physiological | | |
| Cluster | Nanobody | Activity | Cleavage | Cleavage | Inhibitor | Categories | Use |
| 1 | LP8 | Strong | Inhibition | Strong | Less Sensitive | C1-1 | Strong coagulation |
| | LP17 | Inhibition | | Inhibition | | | potentiation, strong |
| | | | | | | | PAR1 cleavage |
| | | | | | | | inhibition, H3 |
| | | | | | | | cleavage inhibition, |
| | | | | | | | with prolonged |
| | | | | | | | activity |
| | LP20 | | Minimal | | | C1-2 | Strong coagulation |
| | | | Change | | | | potentiation, strong |
| | | | | | | | PAR1 cleavage |
| | | | | | | | inhibition, H3 |
| | | | | | | | cleavage |
| | | | | | | | preservation, with |
| | | | | | | | prolonged activity |
| | LP6 | Moderate | Inhibition | | | C1-3 | Moderate |
| | LP13 | Inhibition | | | | | coagulation |
| | LP19 | | | | | | potentiation, strong |
| | LP21 | | | | | | PAR1 cleavage |
| | | | | | | | inhibition, H3 |
| | | | | | | | cleavage inhibition, |
| | | | | | | | with prolonged |
| | | | | | | | activity |
| | LP16 | | Minimal | | | C1-4 | Moderate |
| | | | Change | | | | coagulation |
| | | | | | | | potentiation, strong |
| | | | | | | | PAR1 cleavage |
| | | | | | | | inhibition, H3 |
| | | | | | | | cleavage |
| | | | | | | | preservation, with |
| | | | | | | | prolonged activity |
| | LP1 | Weak | Inhibition | | | C1-5 | Weak coagulation |
| | LP5 | Inhibition | | | | | potentiation, strong |
| | | | | | | | PAR1 cleavage |
| | | | | | | | inhibition, H3 |
| | | | | | | | cleavage inhibition, |
| | | | | | | | with prolonged |
| | | | | | | | activity |
| | LP4 | | Minimal | Moderate | Minimal | C1-6 | Weak coagulation |
| | | | Change | Inhibition | Change | | potentiation, |
| | | | | | | | moderate PAR1 |
| | | | | | | | cleavage inhibition, |
| | | | | | | | H3 cleavage |
| | | | | | | | preservation |
| | LP14 | | | | More Sensitive | C1-7 | Weak coagulation |
| | | | | | | | potentiation, |
| | | | | | | | moderate PAR1 |
| | | | | | | | cleavage inhibition, |
| | | | | | | | H3 cleavage |
| | | | | | | | preservation, with |
| | | | | | | | shortened activity |
| | LP15 | | | | Less Sensitive | C1-8 | Weak coagulation |
| | | | | | | | potentiation, |
| | | | | | | | moderate PAR1 |
| | | | | | | | cleavage inhibition, |
| | | | | | | | H3 cleavage |
| | | | | | | | preservation, with |
| | | | | | | | prolonged activity |
| 2 | LP11 | Strong | Minimal | No | Minimal | C2-1 | Strong coagulation |
| | LP9 | Inhibition | Change | Inhibition | change | | potentiation, no |
| | | | | | | | PAR1 cleavage |
| | | | | | | | inhibition, H3 |
| | | | | | | | cleavage |
| | | | | | | | preservation |
| | LP12 | Moderate | Inhibition | Strong | Less Sensitive | C2-2 | Moderate |
| | | Inhibition | | Inhibition | | | coagulation |
| | | | | | | | potentiation, strong |
| | | | | | | | PAR1 cleavage |
| | | | | | | | inhibition, H3 |
| | | | | | | | cleavage inhibition, |
| | | | | | | | with prolonged |
| | | | | | | | activity |
| | LP7 | Weak | | Moderate | Minimal | C2-3 | Weak coagulation |
| | | Inhibition | | Inhibition | Change | | potentiation, |
| | | | | | | | moderate PAR1 |
| | | | | | | | cleavage inhibition, |
| | | | | | | | H3 cleavage |
| | | | | | | | inhibition |
| | LP18 | | Minimal | No | More Sensitive | C2-4 | Weak coagulation |
| | | | Change | Inhibition | | | potentiation, no |
| | | | | | | | PAR1 cleavage |
| | | | | | | | inhibition, H3 |
| | | | | | | | cleavage |
| | | | | | | | preservation, with |
| | | | | | | | shortened activity |
| 3 | LP2 | Strong | Inhibition | Strong | Less Sensitive | C3-1 | Strong coagulation |
| | | Inhibition | | Inhibition | | | potentiation, strong |
| | | | | | | | PAR1 cleavage |
| | | | | | | | inhibition, H3 |
| | | | | | | | cleavage inhibition, |
| | | | | | | | with prolonged |
| | | | | | | | activity |
| | LP10 | Moderate | | Moderate | Minimal | C3-2 | Moderate |
| | | Inhibition | | Inhibition | change | | coagulation |
| | | | | | | | potentiation, |
| | | | | | | | moderate PAR1 |
| | | | | | | | cleavage inhibition, |
| | | | | | | | H3 cleavage |
| | | | | | | | inhibition |
| | LP3 | | Minimal | No | | C3-3 | Moderate |
| | | | Change | Inhibition | | | coagulation |
| | | | | | | | potentiation, no |
| | | | | | | | PAR1 cleavage |
| | | | | | | | inhibition, H3 |
| | | | | | | | cleavage |
| | | | | | | | preservation |

### Effect of LP11 on Endothelial Cell Barrier Function

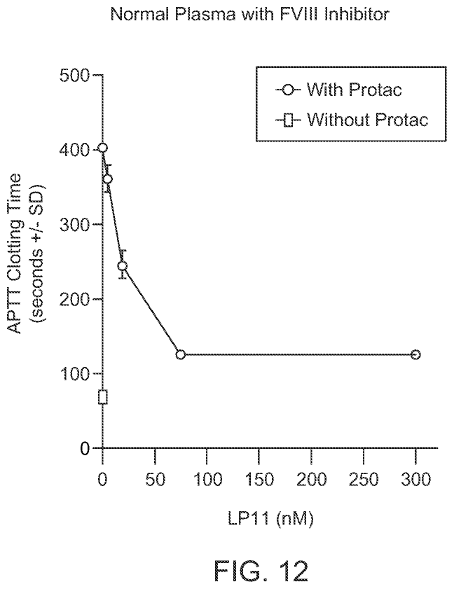

The nanobody LP11 is a top candidate for treating bleeding. LP11 inhibited APC in a dose-dependent manner, shortened the prolonged clotting time in Protac®-APTT clotting assays of FVIII inhibitor plasma (FIG. 12), inhibited anticoagulation, and enhanced the PAR1 R46/R41 cleavage ratio. Thus, LP11 supports pro-hemostatic activity and enhances APC-mediated cytoprotective effects at the same time.

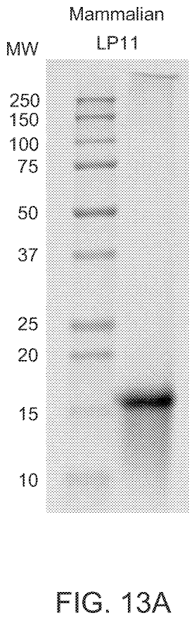

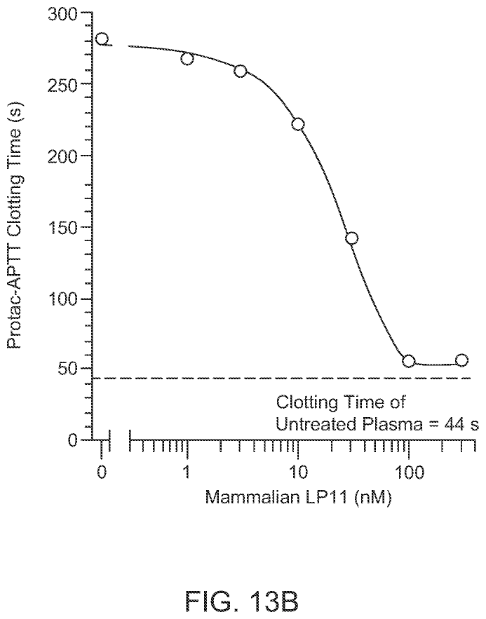

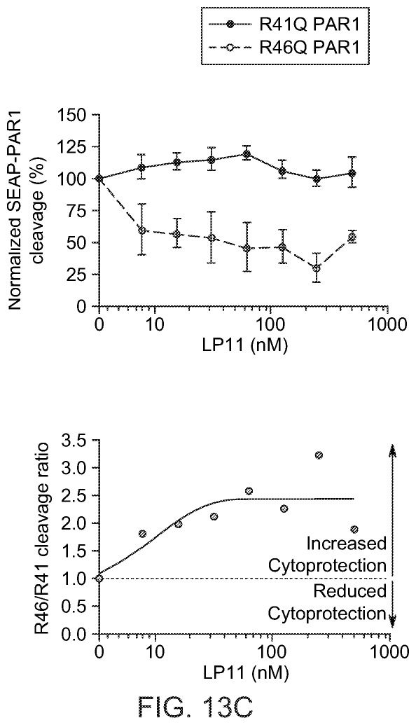

To test the effect of LP11 on endothelial barrier function in vitro, LP11 was expressed in mammalian cells and purified. Characterization of CHO cell-derived LP11 demonstrated that its properties are indistinguishable from E. Coli-derived LP11 (i.e., it inhibits APC's anticoagulant activity, and has minimal effects on APC-mediated PAR1 cleavage at R46 while significantly inhibiting R41 cleavage (FIG. 13)). A classical endothelial barrier function assay was performed using EA.hy926 cells to analyze APC-induced barrier protection against thrombin-induced permeability (18, 39). This assay showed that LP11 has minimal effects on APC's barrier protective function (FIG. 5A-B), which is consistent its minimal effect on APC-mediated PAR1 cleavage at R46. To evaluate the effects of LP11 on the priming of endothelial cell by APC through PAR1 cleavage, we evaluated the endothelial cell layer integrity before barrier disruption was induced by thrombin. APC-priming was observed to induce a dose-dependent transient barrier disruption (FIG. 5C-D) that could be inhibited by the PAR1 antagonists vorapaxar and SCH-79797 (FIG. 5E), consistent with the notion that APC-priming encompasses the effects of APC-mediated cleavage of PAR1 at R41. LP11 inhibited the transient barrier disruption induced by APC-priming in a dose-dependent manner. This was consistent with its inhibition of APC-mediated PAR1 cleavage at R41 (FIG. 5F-G). Most importantly, analysis of the overall barrier function profile showed that the normalized cell index for APC in the presence of LP11 reached higher values compared to APC in the absence of LP11 (FIG. 5H), suggesting that LP11 provides a net enhancement of APC's barrier protective effects. Notably, LP11 alone did not change the electrical resistance of the endothelial layer. Thus, this data demonstrates that preferential inhibition of APC-mediated R41 cleavage over R46 cleavage during APC-priming exerts a cytoprotective effect on endothelial cells.

### Discussion:

The APC pathway, which includes thrombin/thrombomodulin, PC, protein S, EPCR, and PARs, mediates multiple physiological functions including anticoagulant, anti-inflammatory, and cytoprotective functions. The targeting of these proteins may provide clinical benefits for the myriad of pathologies involving this pathway. Currently, KRK-α₁-antitrypsin and 3K3A-APC, two drugs that target different components of this pathway, are under clinical development for hemophilia and ischemic stroke, respectively (40). However, much of the potential for using APC and its substrates as therapeutic targets awaits further exploration. Studies based on single molecule FRET and small angle X-ray scattering have shown that there are limited changes in the overall architecture of PC upon activation, and that these changes are largely conformational changes within the active site region (41, 42). Thus, the identification of antibodies that are specific to non-active sites of APC and do not bind PC presents a challenge. The present work addresses this challenge via construction of a llama antibody library from which 21 nanobodies belonging to 18 families were selected and were further characterized for their binding diversity. In epitope binding studies, only 3 clusters of nanobodies specific for 3 epitope bins were found. This low number of epitope bins points to the limited conformational differences present between non-active site regions of APC and its zymogen PC and agrees with previous reports (41, 42).

Based on the APC functional studies, all 3 epitope bins appear to play multiple roles in anticoagulation, H3 cleavage, and PAR1 cleavage simultaneously, since none of the bins showed an exclusive loss of only one role upon blockade. Nanobodies within Cluster 1 competed with a previously reported antibody, TPP-4885, which targets the APC autolysis loop and potentially also amino acid L429-N431 (8). Thus, nanobodies within Cluster 1 are expected to bind to a similar region. The autolysis loop is a positively charged loop that plays a role in FVa cleavage (43), FVIIIa cleavage (44), and the sensitivity of APC to serpin inhibition (45, 46). Within the autolysis loop, residues R306, K308, K311, R312, and R314 are important for FVa cleavage, while R306, K311 and R314 are important for FVIIIa cleavage. Since all Cluster 1 nanobodies reverse the prolonged Protac®-APTT clotting time to some degree, they are all likely binding to these important arginine and lysine residues on the autolysis loop. The observation that some Cluster 1 nanobodies inhibit H3 and/or PAR1 cleavage could be the result of previously unreported interactions between the autolysis loop with H3 and PAR1. Alternatively, these nanobodies could be binding to regions important for H3 and PAR1 interactions that are proximal but not within the autolysis loop.

Clusters 2 and 3 comprise nanobodies that did not compete with TPP-4885 Fab binding. Among them, LP9 and LP11 inhibited APC's anticoagulant activity without interfering with H3 and PAR1 cleavage. The activity profile of LP9- or LP11-bound APC is similar to that of APC mutants designed to have minimal anticoagulation activity, such as 3K3A-APC and 5A-APC. 3K3A-APC (K191A/K192A/K193A) (47) has amino acid substitutions in loop 37 and 5A-APC (K191A/K192A/K193A/R229A/R230A) (48) has substitutions in both loop 37 and the calcium-binding loop. APC's cytoprotective functions are based on its biased cleavage of PAR1 at R46. R46/R41 cleavage ratio studies on SEAP-PAR1 cell lines show that LP11 increases the R46/R41 cleavage ratio by up to 2.5-fold, which results in overall improvement of endothelial cell barrier function. Preclinical hemophilia and coagulopathy studies have shown that the pro-hemostatic effect of APC inhibition should be elicited without perturbing the cytoprotective function of APC to provide improved outcomes in hemophilic arthropathy (9) and survival outcome in traumatic coagulopathy (28). Thus, the novel properties of LP11 may further improve the outcome of these conditions.

In summary, the llama nanobody library yielded 21 nanobodies that can selectively modulate APC anticoagulation and cytoprotection pathways. These nanobodies are useful as novel therapeutics for the treatment of an array of APC-associated indications including acute bleeding, hemophilia, ischemia, and sepsis.

### REFERENCES

Each patent and non-patent publication cited herein is hereby incorporated herein by reference in its entirety, including without limitation the references listed below.
1. C. T. Esmon, Protein C anticoagulant system—anti-inflammatory effects. Semin Immunopathol 34, 127-132 (2012).
2. J. H. Griffin, B. V. Zlokovic, L. O. Mosnier, Activated protein C: biased for translation. Blood 125, 2898-2907(2015).
3. L. O. Mosnier, J. H. Griffin, in Hemostasis and Thrombosis: Basic Principles and Clinical Practice, V. J. Marder, W. C. Aird, J. S. Bennett, S. Schulman, G. C. White, R. W. Colman, Eds. (2013).
4. C. E. Ettingshausen, S. Halimeh, K. Kurnik, R. Schobess, C. Wermes, R. Junker, W. Kreuz, H. Pollmann, U. Nowak-Göttl, Symptomatic onset of severe hemophilia A in childhood is dependent on the presence of prothrombotic risk factors. Thromb Haemostasis 85, 218-200 (2001).
5. F. Vianello, D. Belvini, F. D. Bello, G. Tagariello, E. Zanon, A. Lombardi, P. Zerbinati, A. Girolami, Mild bleeding diathesis in a boy with combined severe haemophilia B (C10400→T) and heterozygous factor V Leiden. Haemophilia 7, 511-514 (2001).
6. W. Nichols, K. Amano, P. Cacheris, M. Figueiredo, K. Michaelides, R. Schwaab, L. Hoyer, R. Kaufman, D. Ginsburg, Moderation of hemophilia A phenotype by the factor V R506Q mutation. Blood 88, 1183-1187 (1996).
7. B. C. Joseph, B. Y. Miyazawa, C. Esmon, M. J. Cohen, A. von Drygalski, L. O. Mosnier, An engineered activated factor V for the prevention and treatment of acute traumatic coagulopathy and bleeding in mice. Blood Adv 6, 959-969 (2021).
8. X.-Y. Zhao, A. Wilmen, D. Wang, X. Wang, M. Bauzon, J.-Y. Kim, L. Linden, L. Li, U. Egner, T. Marquardt, D. Moosmayer, J. Tebbe, J. M. Glück, P. Ellinger, K. McLean, S. Yuan, S. Yegneswaran, X. Jiang, V. Evans, J.-M. Gu, D. Schneider, Y. Zhu, Y. Xu, C. Mallari, A. Hesslein, Y. Wang, N. Schmidt, K. Gutberlet, C. Ruehl-Fehlert, A. Freyberger, T. Hermiston, C. Patel, D. Sim, L. O. Mosnier, V. Laux, Targeted inhibition of activated protein C by a non-active-site inhibitory antibody to treat hemophilia. Nat Commun 11, 2992 (2020).
9. J. Magisetty, V. Kondreddy, S. Keshava, K. Das, C. T. Esmon, U. R. Pendurthi, L. V. M. Rao, Selective inhibition of activated protein C anticoagulant activity protects against hemophilic arthropathy. Blood 139, 2830-2841 (2022).
10. M. Jiang, F. Yang, Y. Jiang, L. Cheng, J. Han, J. Yi, G. Zhang, Z. Ma, L. Cao, B. Zuo, L. Zhou, L. Huang, S. Niu, Z. Xia, X. Zhou, X. Bai, N. L. Esmon, C. Ruan, L. Xia, Y. Han, C. Esmon, D. Wu, J. Xu, Blocking human protein C anticoagulant activity improves clotting defects of hemophilia mice expressing human protein C. Blood Adv 6, 3304-3314 (2022).
11. S. G. I. Polderdijk, T. E. Adams, L. Ivanciu, R. M. Camire, T. P. Baglin, J. A. Huntington, Design and characterization of an APC-specific serpin for the treatment of hemophilia. Blood 129, 105-113 (2017).
12. T. Baglin, A. Koch, I. Mocanu, L. Makhaldiani, J. A. Huntington, Serpinpc in Persons with Severe Hemophilia (PwH): Updated Results from a Multi-Center, Multi-Part, First-in-Human Study. Blood 140, 460-461 (2022).
13. M. Thiyagarajan, J. A. Fernandez, S. M. Lane, J. H. Griffin, B. V. Zlokovic, Activated protein C promotes neovascularization and neurogenesis in postischemic brain via protease-activated receptor 1. J Neurosci 28, 12788-12797 (2008).
14. H. Guo, Z. Zhao, Q. Yang, M. Wang, R. D. Bell, S. Wang, N. Chow, T. P. Davis, J. H. Griffin, S. A. Goldman, B. V. Zlokovic, An activated protein C analog stimulates neuronal production by human neural progenitor cells via a PAR1-PAR3-S1PR1-Akt pathway. J Neurosci 33, 6181-6190 (2013).
15. K. McKelvey, C. J. Jackson, M. Xue, Activated protein C: A regulator of human skin epidermal keratinocyte function. World J Biological Chem 5, 169-79 (2014).
16. J. Xu, X. Zhang, R. Pelayo, M. Monestier, C. T. Ammollo, F. Semeraro, F. B. Taylor, N. Esmon, F. Lupu, C. T. Esmon, Extracellular histones are major mediators of death in sepsis. Nat Med 15, 1318-1321 (2009).
17. P. Szatmary, W. Huang, D. Criddle, A. Tepikin, R. Sutton, Biology, role and therapeutic potential of circulating histones in acute inflammatory disorders. J Cell Mol Med 22, 4617-4629 (2018).
18. L. O. Mosnier, R. K. Sinha, L. Burnier, E. A. Bouwens, J. H. Griffin, Biased agonism of protease-activated receptor 1 by activated protein C caused by noncanonical cleavage at Arg46. Blood 120, 5237-5246 (2012).
19. R. K. Sinha, Y. Wang, Z. Zhao, X. Xu, L. Burnier, N. Gupta, J. A. Fernández, G. Martin, S. Kupriyanov, L. O. Mosnier, B. V. Zlokovic, J. H. Griffin, PAR1 biased signaling is required for activated protein C in vivo benefits in sepsis and stroke. Blood 131, 1163-1171 (2018).
20. S. W. Gal, S. Amontov, P. T. Urvil, D. Vishnuvardhan, F. Nishikawa, P. K. R. Kumar, S. Nishikawa, Selection of a RNA aptamer that binds to human activated protein C and inhibits its protease function. Eur J Biochem 252, 553-562 (2017).
21. J. Müller, B. Isermann, C. Dücker, M. Salehi, M. Meyer, M. Friedrich, T. Madhusudhan, J. Oldenburg, G. Mayer, B. Pötzsch, An exosite-specific ssDNA aptamer inhibits the anticoagulant functions of activated protein C and enhances inhibition by protein C inhibitor. Chem Biol 16, 442-451 (2009).
22. N. S. Hamedani, H. Rühl, J. J. Zimmermann, T. Heiseler, J. Oldenburg, G. Mayer, B. Pötzsch, J. Müller, In vitro evaluation of aptamer-based reversible inhibition of anticoagulant activated protein C as a novel supportive hemostatic approach. Nucleic Acid Ther 26, 355-362 (2016).
23. N. S. Hamedani, J. Müller, F. Tolle, H. Rühl, B. Pezeshkpoor, K. Liphardt, J. Oldenburg, G. Mayer, B. Pötzsch, Selective modulation of the protease activated protein C using exosite inhibiting aptamers. Nucleic Acid Ther 30, 276-288 (2020).
24. G. R. Bernard, J.-L. Vincent, P.-F. Laterre, S. P. LaRosa, J.-F. Dhainaut, A. Lopez-Rodriguez, J. S. Steingrub, G. E. Garber, J. D. Helterbrand, E. W. Ely, C. J. Fisher, Efficacy and safety of recombinant human activated protein C for severe sepsis. New Engl J Medicine 344, 699-709 (2001).
25. V. M. Ranieri, B. T. Thompson, P. S. Barie, J.-F. Dhainaut, I. S. Douglas, S. Finfer, B. Gårdlund, J. C. Marshall, A. Rhodes, A. Artigas, D. Payen, J. Tenhunen, H. R. Al-Khalidi, V. Thompson, J. Janes, W. L. Macias, B. Vangerow, M. D. Williams, P.-S. S. Group, Drotrecogin alfa (activated) in adults with septic shock. New Engl J Medicine 366, 2055-2064 (2012).
26. P. Lyden, H. Levy, S. Weymer, K. Pryor, W. Kramer, J. Griffin, T. Davis, B. Zlokovic, Phase 1 Safety, tolerability and pharmacokinetics of 3K3A-APC in healthy adult volunteers. Curr Pharm Design 19, 7479-7485 (2014).
27. P. Lyden, K. E. Pryor, C. S. Coffey, M. Cudkowicz, R. Conwit, A. Jadhav, R. N. Sawyer, J. Claassen, O. Adeoye, S. Song, P. Hannon, N. S. Rost, A. Hinduja, M. Torbey, J. Lee, C. Benesch, M. Rippee, M. Rymer, M. T. Froehler, E. C. Haley, M. Johnson, J. Yankey, K. Magee, J. Qidwai, H. Levy, E. M. Haacke, M. Fawaz, T. P. Davis, A. W. Toga, J. H. Griffin, B. V. Zlokovic, the N. C. T. N. N. Investigators, Final results of the RHAPSODY trial: a multi-center, phase 2 trial using a continual reassessment method to determine the safety and tolerability of 3K3A-APC, a recombinant variant of human activated protein C, in combination with tissue plasminogen activator, mechanical thrombectomy or both in moderate to severe acute ischemic stroke. Ann Neurol 85, 125-136 (2019).
28. B. B. Chesebro, P. Rahn, M. Carles, C. T. Esmon, J. Xu, K. Brohi, D. Frith, J.-F. Pittet, M. J. Cohen, Increase in activated protein C mediates acute traumatic coagulopathy in mice. Shock 32, 659-665 (2009).
29. J. XU, Y. JI, X. ZHANG, M. DRAKE, C. T. Esmon, Endogenous activated protein C signaling is critical to protection of mice from lipopolysaccaride-induced septic shock. J Thromb Haemost 7, 851-856 (2009).
30. C. Hamers-Casterman, T. Atarhouch, S. Muyldermans, G. Robinson, C. Hammers, E. B. Songa, N. Bendahman, R. Hammers, Naturally occurring antibodies devoid of light chains. Nature 363, 446-448 (1993).
31. S. Muyldermans, Nanobodies: Natural Single-Domain Antibodies. Annu Rev Biochem 82, 775-797 (2013).
32. I. Peyron, C. Kizlik-Masson, M. Dubois, S. Atsou, S. Ferriere, C. V. Denis, P. J. Lenting, C. Casari, O. D. Christophe, Camelid-derived single-chain antibodies in hemostasis: Mechanistic, diagnostic, and therapeutic applications. Res Pract Thrombosis Haemostasis 4, 1087-1110(2020).
33. B. Rissiek, F. Koch-Nolte, T. Magnus, Nanobodies as modulators of inflammation: potential applications for acute brain injury. Front Cell Neurosci 8, 344 (2014).
34. E. Ruiz-López, A. J. Schuhmacher, Transportation of single-domain antibodies through the blood-brain barrier. Biomol 11, 1131 (2021).
35. E. D. Genst, K. Silence, K. Decanniere, K. Conrath, R. Loris, J. Kinne, S. Muyldermans, L. Wyns, Molecular basis for the preferential cleft recognition by dromedary heavy-chain antibodies. Proc National Acad Sci 103, 4586-4591 (2006).
36. A. Desmyter, T. R. Transue, M. A. Ghahroudi, M.-H. D. Thi, F. Poortmans, R. Hamers, S. Muyldermans, L. Wyns, Crystal structure of a camel single-domain VH antibody fragment in complex with lysozyme. Nat Struct Biol 3, 803-811 (1996).
37. M. Lauwereys, M. A. Ghahroudi, A. Desmyter, J. Kinne, W. Holzer, E. D. Genst, L. Wyns, S. Muyldermans, Potent enzyme inhibitors derived from dromedary heavy-chain antibodies. Embo J 17, 3512-3520 (1998).
38. L. Burnier, L. O. Mosnier, Novel mechanisms for activated protein C cytoprotective activities involving noncanonical activation of protease-activated receptor 3. Blood 122, 807-816 (2013).
39. F. Stavenuiter, L. O. Mosnier, Noncanonical PAR3 activation by factor Xa identifies a novel pathway for Tie2 activation and stabilization of vascular integrity. Blood 124, 3480-3489 (2014).
40. J. H. Griffin, B. V. Zlokovic, L. O. Mosnier, Activated protein C, protease activated receptor 1, and neuroprotection. Blood 132, 159-169 (2018).
41. B. M. Stojanovski, L. A. Pelc, X. Zuo, E. D. Cera, Zymogen and activated protein C have similar structural architecture. J Biological Chem 295, 15236-15244 (2020).
42. P. E. Bock, P. Panizzi, I. M. A. Verhamme, Exosites in the substrate specificity of blood coagulation reactions. J Thromb Haemost 5, 81-94 (2007).
43. A. J. Gale, M. J. Heeb, J. H. Griffin, The autolysis loop of activated protein C interacts with factor Va and differentiates between the Arg506 and Arg306 cleavage sites. Blood 96, 585-593 (2000).
44. T. J. Cramer, A. J. Gale, Function of the activated protein C (APC) autolysis loop in activated FVIII inactivation. Brit J Haematol 153, 644-654 (2011).
45. L. Yang, C. Manithody, A. R. Rezaie, The functional significance of the autolysis loop in protein C and activated protein C. Thromb Haemostasis 94, 60-68 (2005).
46. L. Shen, B. Villoutreix, B. Dahlback, Interspecies loop grafting in the protease domain of human protein C yielding enhanced catalytic and anticoagulant activity. Thromb Haemostasis 82, 1078-1087 (1999).
47. L. O. Mosnier, A. J. Gale, S. Yegneswaran, J. H. Griffin, Activated protein C variants with normal cytoprotective but reduced anticoagulant activity. Blood 104, 1740-1744 (2004).
48. L. O. Mosnier, X. V. Yang, J. H. Griffin, Activated protein C mutant with minimal anticoagulant activity, normal cytoprotective activity, and preservation of thrombin activable fibrinolysis inhibitor-dependent cytoprotective functions. J Biol Chem 282, 33022-33033 (2007).
49. R. Suzuki, H. Shimodaira, Pvclust: an R package for assessing the uncertainty in hierarchical clustering. Bioinformatics 22, 1540-1542 (2006).

1. An isolated antibody comprising a CDR3 comprising an amino acid sequence selected from the group consisting of SEQ ID NOs: 317-474, wherein said antibody specifically binds to activated protein C (APC) and minimally binds to unactivated protein C (PC).

2. The antibody of claim 1, wherein the antibody further comprises (a) a CDR1 comprising an amino acid sequence selected from the group consisting of SEQ ID NOs: 1-158, (b) a CDR2 comprising an amino acid sequence selected from the group consisting of SEQ ID NOs: 159-316, or (c) both a CDR1 comprising an amino acid sequence selected from the group consisting of SEQ ID NOs: 1-158 and a CDR2 comprising an amino acid sequence selected from the group consisting of SEQ ID NOs: 159-316.

3. The antibody of claim 2, wherein the antibody comprises the CDR1, CDR2, and CDR3 of a heavy chain variable region (V_{H}) having an amino acid sequence selected from the group consisting of SEQ ID NOs: 475-632.

4. The antibody of claim 3, wherein the antibody comprises the:
1) CDR1 of SEQ ID NO: 45, CDR2 of SEQ ID NO: 203, and CDR3 of SEQ ID NO: 361;
2) CDR1 of SEQ ID NO: 54, CDR2 of SEQ ID NO: 212, and CDR3 of SEQ ID NO: 370;
3) CDR1 of SEQ ID NO: 61, CDR2 of SEQ ID NO: 219, and CDR3 of SEQ ID NO: 377;
4) CDR1 of SEQ ID NO: 74, CDR2 of SEQ ID NO: 232, and CDR3 of SEQ ID NO: 390;
5) CDR1 of SEQ ID NO: 82, CDR2 of SEQ ID NO: 240, and CDR3 of SEQ ID NO: 398;
6) CDR1 of SEQ ID NO: 84, CDR2 of SEQ ID NO: 242, and CDR3 of SEQ ID NO: 400;
7) CDR1 of SEQ ID NO: 88, CDR2 of SEQ ID NO: 246, and CDR3 of SEQ ID NO: 404;
8) CDR1 of SEQ ID NO: 90, CDR2 of SEQ ID NO: 248, and CDR3 of SEQ ID NO: 406;
9) CDR1 of SEQ ID NO: 95, CDR2 of SEQ ID NO: 253, and CDR3 of SEQ ID NO: 411;
10) CDR1 of SEQ ID NO: 101, CDR2 of SEQ ID NO: 259, and CDR3 of SEQ ID NO: 417;
11) CDR1 of SEQ ID NO: 103, CDR2 of SEQ ID NO: 261, and CDR3 of SEQ ID NO: 419;
12) CDR1 of SEQ ID NO: 105, CDR2 of SEQ ID NO: 263, and CDR3 of SEQ ID NO: 421;
13) CDR1 of SEQ ID NO: 109, CDR2 of SEQ ID NO: 267, and CDR3 of SEQ ID NO: 425;
14) CDR1 of SEQ ID NO: 116, CDR2 of SEQ ID NO: 274, and CDR3 of SEQ ID NO: 432;
15) CDR1 of SEQ ID NO: 142, CDR2 of SEQ ID NO: 300, and CDR3 of SEQ ID NO: 458;
16) CDR1 of SEQ ID NO: 144, CDR2 of SEQ ID NO: 302, and CDR3 of SEQ ID NO: 460;
17) CDR1 of SEQ ID NO: 146, CDR2 of SEQ ID NO: 304, and CDR3 of SEQ ID NO: 462;
18) CDR1 of SEQ ID NO: 147, CDR2 of SEQ ID NO: 305, and CDR3 of SEQ ID NO: 463;
19) CDR1 of SEQ ID NO: 149, CDR2 of SEQ ID NO: 307, and CDR3 of SEQ ID NO: 465;
20) CDR1 of SEQ ID NO: 150, CDR2 of SEQ ID NO: 308, and CDR3 of SEQ ID NO: 466; or
21) CDR1 of SEQ ID NO: 157, CDR2 of SEQ ID NO: 315, and CDR3 of SEQ ID NO: 473.

5. An isolated antibody comprising a V_{H} comprising an amino acid sequence selected from the group consisting of SEQ ID NOs: 475-632, wherein said antibody specifically binds to APC and minimally binds to PC.

6. The antibody of claim 1, wherein said antibody binds to an exosite of APC.

7. The antibody of claim 1, wherein the antibody is an antibody selected from the group consisting of an IgG1 antibody, an IgG2 antibody, an IgG3 antibody, an IgG4 antibody, an IgM antibody, an IgA1 antibody, an IgA2 antibody, a secretory IgA antibody, an IgD antibody, an IgE antibody, and an antigen-binding fragment thereof.

8. The antibody of claim 1, wherein the antibody is a nanobody.

9. The antibody of claim 8, wherein the antibody is a V_{H}H antibody.

10. The antibody of claim 1, wherein the antibody:
a) enhances, minimally affects, or inhibits the anticoagulant activity of APC;
b) enhances, minimally affects, or inhibits APC-mediated histone cleavage;
c) enhances, minimally affects, or inhibits APC-mediated protease-activated receptor 1 (PAR1) cleavage at residue R46;
d) enhances, minimally affects, or inhibits APC-mediated PAR1 cleavage at residue R41;
e) increases, minimally affects, or decreases the ratio of APC-mediated PAR1 cleavage at residue R46 to APC-mediated PAR1 cleavage at residue R41;
f) enhances, minimally affects, or inhibits the endothelial barrier protective activity of APC;
g) increases, minimally affects, or decreases the plasma half-life of APC; or
h) any combination thereof.

11. An isolated antibody that specifically binds to APC, minimally binds to PC, and competes with the antibody of claim 1 for binding to APC.

12. A nucleic acid encoding the antibody of claim 1.

13. A vector comprising the nucleic acid of claim 12.

14. A host cell comprising the nucleic acid of claim 12.

15. A method of producing an antibody, the method comprising:
a) culturing the host cell of claim 14 under conditions that result in production of the antibody; and
b) isolating the antibody from the host cell.

16. A pharmaceutical composition comprising the antibody of claim 1 and a pharmaceutically acceptable carrier.

17. A method for treating or preventing a condition in a subject, the method comprising administering a therapeutically effective amount of the antibody of claim 1.

18. The method of claim 17, wherein the condition is a condition that can be treated or prevented by enhancing or inhibiting the anticoagulant function of APC.

19. The method of claim 17, wherein the condition is selected from the group consisting of a hemorrhage, a contusion, a burn, gastrointestinal bleeding, uncontrolled bleeding, bleeding due to a transplantation or resection procedure, bleeding due to a surgery, bleeding due to a traumatic injury, variceal bleeding, bleeding in cirrhosis, thrombocytopenia, idiopathic thrombocytopenic purpura, hemophilia, aortic aneurysm, over-administration of an anticoagulant or antithrombotic, menorrhagia, deficiency of a clotting factor, Glanzmann's Thrombasthenia, and Bernard-Soulier syndrome.

20. The method of claim 17, wherein the condition is a condition that can be treated or prevented by enhancing or inhibiting one or more cytoprotective function of APC.

21. The method of claim 20, wherein the condition is selected from the group consisting of sepsis, inflammation in acute ischemic disease, coronavirus disease 2019 (COVID-19), diabetes, diabetic nephropathy, diabetic ulcers, wounds, amyotrophic lateral sclerosis (ALS), multiple sclerosis, central nervous system injury, ischemic stroke, Alzheimer's disease, acute kidney injury, a lung disorder, acute pancreatitis, a cancer, an inflammatory disease, and an autoimmune disease.

22. The antibody of claim 1 for use as a medicament for treating or preventing a condition.

23. The antibody of claim 22, wherein the condition is a condition that can be treated or prevented by enhancing or inhibiting the anticoagulant function of APC.

24. The antibody of claim 23, wherein the condition is selected from the group consisting of a hemorrhage, a contusion, a burn, gastrointestinal bleeding, uncontrolled bleeding, bleeding due to a transplantation or resection procedure, bleeding due to a surgery, bleeding due to a traumatic injury, variceal bleeding, bleeding in cirrhosis, thrombocytopenia, idiopathic thrombocytopenic purpura, hemophilia, aortic aneurysm, over-administration of an anticoagulant or antithrombotic, menorrhagia, deficiency of a clotting factor, Glanzmann's Thrombasthenia, and Bernard-Soulier syndrome.

25. The antibody of claim 22, wherein the condition is a condition that can be treated or prevented by enhancing or inhibiting one or more cytoprotective function of APC.

26. The antibody of claim 25, wherein the condition is selected from the group consisting of sepsis, inflammation in acute ischemic disease, COVID-19, diabetes, diabetic nephropathy, diabetic ulcers, wounds, ALS, multiple sclerosis, central nervous system injury, ischemic stroke, Alzheimer's disease, acute kidney injury, a lung disorder, acute pancreatitis, a cancer, an inflammatory disease, and an autoimmune disease.
